SEQUENCE LISTING (201 sequences)

SEQ ID NO: 1 - protein, 219 aa, Yersinia enterocolitica
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATGSGPLRGSITQCQGLMQFCGGELQAEASAILNTPVCGIPFSQWGTVGGAASAYVASGVDLTQAANEIKGLGQQMQQLLSLM

SEQ ID NO: 2 - protein, 138 aa, Yersinia enterocolitica
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFAT

SEQ ID NO: 3 - protein, 169 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRFEKLGPEQKLISEEDLNSAVDHHHHHH

SEQ ID NO: 4 - protein, 348 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMQILNKILPQVEFAIPRPSFDSLSRNKLVKKILSVFNLKQRFPQKNFGCPVNINKIRDSVIDKIKDSNSGNQLFCWMSQERTTYVSSMINRSIDEMAIHNGVVLTSDNKRNIFAAIEKKFPDIKLDEKSAQTSISHTALNEIASSGLRAKILKRYSSDMDLFNTQMKDLTNLVSSSVYDKIFNESTKVLQIEISAEVLKAVYRQSNTN

SEQ ID NO: 5 - protein, 380 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEVTNITLSTQHYRIHRSDVEPVKEKTTEKDIFAKSITAVRNSFISLSTSLSDRFSLHQQTDIPTTHFHRGNASEGRAVLTSKTVKDFMLQKLNSLDIKGNASKDPAYARQTCEAILSAVYSNNKDQCCKLLISKGVSITPFLKEIGEAAQNAGLPGEIKNGVFTPGGAGANPFVVPLIASASIKYPHMFINHNQQVSFKAYAEKIVMKEVTPLFNKGTMPTPQQFQLTIENIANKYLQNAS

SEQ ID NO: 6 - protein, 701 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMQIQSFYHSASLKTQEAFKSLQKTLYNGMQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFCGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKREIISLHQTHMLSAPGSLPDSGGQKIFQKVLLNSGNLEIQKQNTGGAGNKVMKNLSPEVLNLSYQKRVGDENIWQSVKGISSLITS

SEQ ID NO: 7 - protein, 383 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRFEMPIKKPCLKLNLDSLNVVRSEIPQMLSANERLKNNFNILYNQIRQYPAYYFKVASNVPTYSDICQSFSVMYQGFQIVNHSGDVFIHACRENPQSKGDFVGDKFHISIAREQVPLAFQILSGLLFSEDSPIDKWKITDMNRVSQQSRVGIGAQFTLYVKSDQECSQYSALLLHKIRQFIMCLESNLLRSKIAPGEYPASDVRPEDWKYVSYRNELRSDRDGSERQEQMLREEPFYRLMIE

SEQ ID NO: 8 - protein, 685 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMLKYEERKLNNLTLSSFSKVGVSNDARLYIAKENTDKAYVAPEKFSSKVLTWLGKMPLFKNTEVVQKHTENIRVQDQKILQTFLHALTEKYGETAVNDALLMSRINMNKPLTQRLAVQITECVKAADEGFINLIKSKDNVGVRNAALVIKGGDTKVAEKNNDVGAESKQPLLDIALKGLKRTLPQLEQMDGNSLRENFQEMASGNGPLRSLMTNLQNLNKIPEAKQLNDYVTTLTNIQVGVARFSQWGTCGGEVERWVDKASTHELTQAVKKIHVIAKELKNVTAELEKIEAGAPMPQTMSGPTLGLARFAVSSIPINQQTQVKLSDGMPVPVNTLTFDGKPVALAGSYPKNTPDALEAHMKMLLEKECSCLVVLTSEDQMQAKQLPPYFRGSYTFGEVHTNSQKVSSASQGEAIDQYNMQLSCGEKRYTIPVLHVKNWPDHQPLPSTDQLEYLADRVKNSNQNGAPGRSSSDKHLPMIHCLGGVGRTGTMAAALVLKDNPHSNLEQVRADFRDSRNNRMLEDASQFVQLKAMQAQLLMTTAS

SEQ ID NO: 9 - protein, 678 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMHITNLGLHQVSFQSGDSYKGAEETGKHKGVSVISYQRVKNGERNKGIEALNRLYLQNQTSLTGKSLLFARDKAEVFCEAIKLAGGDTSKIKAMMERLDTYKLGEVNKRHINELNKVISEEIRAQLGIKNKKELQTKIKQIFTDYLNNKNWGPVNKNISHHGKNYSFQLTPASHMKIGNKNIFVKEYNGKGICCASTRERDHIANMWLSKVVDDEGKEIFSGIRHGVISAYGLKKNSSERAVAARNKAEELVSAALYSRPELLSQALSGKTVDLKIVSTSLLTPTSLTGGEESMLKDQVSALKGLNSKRGGPTKLLIRNSDGLLKEVSVNLKVVTFNFGVNELALKMGLGWRNVDKLNDESICSLLGDNFLKNGVIGGWAAEAIEKNPPCKNDVIYLANQIKEIVNNKLQKNDNGEPYKLSQRVTLLAYTIGAVPCWNCKSGKDRTGMQDAEIKREIIRKHETGQFSQLNSKLSSEEKRLFSTILMNSGNMEIQEMNTGVPGNKVMKKLPLSSLELSYSERIGDPKIWNMVKGYSSFV

SEQ ID NO: 10 - protein, 446 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMPKAKAKTKNTEIISPHHYVYPNTTTLKNKYGIKNLNAFLEKCSHDTAKAMINLREESLPEYFDTAYLCHIHQQLFKNTFEWAGYLRHIPFTFADGTTAAMPEMKRTGWKNAFAIGDEIQEGLQRLDQTLAEKNNLQGLTREEFNSEAIELFNSLNQLHPFREGNGRTQRLFFENLAKAAGHQLNFSLITKERMMVASVAVAENGDLEPMQHLFEDISNPEKIRLLKEFMHTMKNTGRNVNDRPVMVAKEGETYTGTYRGAGLEGFALNVKGAYIIGNIDHLPPEQLKILKPGDKITFTAPKAE

SEQ ID NO: 11 - protein, 284 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESREGNGRTQRLFFENLAKAAGHQLNFSLITKERMMVASVAVAENGDLEPMQHLFEDISNPEKIRLLKEFMHTMKNTGRNVNDRPVMVAKEGETYTGTYRGAGLEGFALNVKGAYIIGNIDHLPPEQLKILKPGDKITFTAPKAE

SEQ ID NO: 12 - protein, 672 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMSLLNCENSCGSSQSSSDCCAAMAASCSAAVKDDSVSGSASTGNLSSSFMEEIQGYDVEFDPPLESKYECPICLMALREAVQTPCGHRFCKACIIKSIRDAGHKCPVDNEILLENQLFPDNFAKREILSLTVKCPNKGCLQKMELRHLEDHQVHCEFALVNCPQCQRPFQKCQVNTHIIEDCPRRQVSCVNCAVSMAYEEKEIHDQSCPLANIICEYCGTILIREQMPNHYDLDCPTAPIPCTFSVFGCHQKMQRNHLARHLQENTQLHMRLLAQAVHNVNLALRPCDAASPSRGCRPEDPNYEETIKQLESRLVRQDHQIRELTAKMETQSMYVGELKRTIRTLEDKVAEMEAQQCNGIYIWKIGKFGMHLKSQEEERPVVIHSPGFYTGRPGYKLCMRLHLQLPTAQRCANYISLFVHTMQGEYDSHLPWPFQGTIRLTILDQSEALIRQNHEEVMDAKPELLAFQRPTIPRNPKGFGYVTFMHLEALRQGTFIKDDTLLVRCEVSTRFDMGGLRKEGFQPRSTDAGV

SEQ ID NO: 13 - protein, 326 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTSFEDADTEETVTCLQMTVYHPGQLQCGIFQSISFNREKLPSSEVVKFGRNSNICHYTFQDKQVSRVQFSLQLFKKFNSSVLSFEIKNMSKKTNLIVDSRELGYLNKMDLPYRCMVRFGEYQFLMEKEDGESLEFFETQFILSPRSLLQENNWPPHRPIPEYGTYSLCSSQSSSPTEMDENES

SEQ ID NO: 14 - protein, 437 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKM

SEQ ID NO: 15 - protein, 347 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMALQLSREQGITLRGSAEIVAEFFSFGINSILYQRGIYPSETFTRVQKYGLTLLVTTDLELIKYLNNVVEQLKDWLYKCSVQKLVVVISNIESGEVLERWQFDIECDKTAKDDSAPREKSQKAIQDEIRSVIRQITATVTFLPLLEVSCSFDLLIYTDKDLVVPEKWEESGPQFITNSEEVRLRSFTTTIHKVNSMVAYKIPVND

SEQ ID NO: 16 - protein, 298 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 17 - protein, 280 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMREENKGMPSGGGSDEGLASAAARGLVEKVRQLLEAGADPNGVNRFGRRAIQVMMMGSARVAELLLLHGAEPNCADPATLTRPVHDAAREGFLDTLVVLHRAGARLDVRDAWGRLPVDLAEERGHRDVAGYLRTATGD

SEQ ID NO: 18 - protein, 310 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMAEPWGNELASAAARGDLEQLTSLLQNNVNVNAQNGFGRTALQVMKLGNPEIARRLLLRGANPDLKDRTGFAVIHDAARAGFLDTLQALPEFQADVNIEDNEGNLPLHLAAKEGHLRVVEFLVKHTASNVGHRNHKGDTACDLARLYGRNEVVSLMQANGAGGATNLQ

SEQ ID NO: 19 - protein, 329 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMDFNRNFDHIPHTSLVLLSFLNQKDCQNGESGRVFDYREDNLSTNHIDSDGDIETDGHSPPATYRDLLHELQHEVQPGLSVNAEEARAAREMAAELIRIADLLEQSVLSQAAESLTKKLRSFQEQVWASHLSKGVQTLLQHVAAAKEFKKELVEMAFTFMLMKTVCERTPDFLFGLYGTVVQFFGSN

SEQ ID NO: 20 - protein, 273 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRGHSPPATYRDLLHELQHEVQPGLSVNAEEARAAREMAAELIRIADLLEQSVLSQAAESLTKKLRSFQEQVWASHLSKGVQTLLQHVAAAKEFKKELVEMAFTFMLMKTVCERTPDFLFGLYGTVVQFFGSN

SEQ ID NO: 21 - protein, 318 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMSDTSREQTLANGPASQGSGESTGGGVVLPAGHFDFPQPGEGDPLRGGISMSNNQSRSPMNRTFSRSSSGYFSVDSDSVPGSPLMPNISEAQDGQNDEVWLSEHSHQHLQMAAPVAALPPEMVVARELRRIGDEFNRLYCEAGAGVNQLRAPNEHAIVLWMNVIIGRLVHFFLRRR

SEQ ID NO: 22 - protein, 289 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETD

SEQ ID NO: 23 - protein, 242 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 24 - protein, 337 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 25 - protein, 277 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 26 - protein, 327 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEQAIKCVVVGDGAVGKTCLLISYTTNAFPGEYIPTVFDNYSANVMVDGKPVNLGLWDTAGEEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRK

SEQ ID NO: 27 - protein, 333 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMAAIRKKLVIVGDGACGKTCLLIVFSKDQFPEVYVPTVFENYVADIEVDGKQVELALWDTAGLEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 28 - protein, 350 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 29 - protein, 308 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSTAPSQ

SEQ ID NO: 30 - protein, 309 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMSGVVGPMQEPGALDVGGLRSQRQKWFQCFDGITSILFMVSSSEYDQVLMEDRRTNRLVESMNIFETIVNNKLFFNVSIILFLNKMDLLVEKVKTVSIKKHFPDFRGDPHRLEDVQRYLVQCFDRKRRNRSKPLFHHFTTAIDTENVRFVFHAVKDTILQENLKDIMLQ

SEQ ID NO: 31 - protein, 259 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 32 - protein, 458 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRKMMKMETDKIMDETNSNAQAFTTTMLYDPVRKKDSSPTYQTERELCFQYFTQWSESGQVDFVEHLLSRMCHYQHGQINAYLKPMLQRDFITLLPIKGLDHIAENILSYLDAESLKSSELVCKEWLRVISEGMLWKKLIERKVRTDSLWRGLAERRNWMQYLFKPRPGQTQRPHSFHRELFPKIMNDIDSIENNWRTGRHMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 33 - protein, 470 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRPPKKKRKVQFKMMKMETDKIMDETNSNAQAFTTTMLYDPVRKKDSSPTYQTERELCFQYFTQWSESGQVDFVEHLLSRMCHYQHGQINAYLKPMLQRDFITLLPIKGLDHIAENILSYLDAESLKSSELVCKEWLRVISEGMLWKKLIERKVRTDSLWRGLAERRNWMQYLFKPRPGQTQRPHSFHRELFPKIMNDIDSIENNWRTGRHLEMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS

SEQ ID NO: 34 - protein, 466 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRKMMKMETDKIMDETNSNAQAFTTTMLYDPVRKKDSSPTYQTERELCFQYFTQWSESGQVDFVEHLLSRMCHYQHGQINAYLKPMLQRDFITLLPIKGLDHIAENILSYLDAESLKSSELVCKEWLRVISEGMLWKKLIERKVRTDSLWRGLAERRNWMQYLFKPRPGQTQRPHSFHRELFPKIMNDIDSIENNWRTGRHMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSPPKKKRKV

SEQ ID NO: 35 - protein, 246 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVAD

SEQ ID NO: 36 - protein, 465 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMPRPKLKSDDEVLEAATVVLKRCGPIEFTLSGVAKEVGLSRAALIQRFTNRDTLLVRMMERGVEQVRHYLNAIPIGAGPQGLWEFLQVLVRSMNTRNDFSVNYLISWYELQVPELRTLAIQRNRAVVEGIRKRLPPGAPAAAELLLHSVIAGATMQWAVDPDGELADHVLAQIAAILCLMFPEHDDFQLLQAHASAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 37 - protein, 381 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 38 - protein, 402 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRENLYFQSENLYFQSPPKKKRKVVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 39 - protein, 402 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRENLYFQSENLYFQSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKPPKKKRKV

SEQ ID NO: 40 - protein, 324 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRENLYFQSENLYFQSMAEPWGNELASAAARGDLEQLTSLLQNNVNVNAQNGFGRTALQVMKLGNPEIARRLLLRGANPDLKDRTGFAVIHDAARAGFLDTLQALPEFQADVNIEDNEGNLPLHLAAKEGHLRVVEFLVKHTASNVGHRNHKGDTACDLARLYGRNEVVSLMQANGAGGATNLQ

SEQ ID NO: 41 - protein, 479 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRENLYFQSENLYFQSMPRPKLKSDDEVLEAATVVLKRCGPIEFTLSGVAKEVGLSRAALIQRFTNRDTLLVRMMERGVEQVRHYLNAIPIGAGPQGLWEFLQVLVRSMNTRNDFSVNYLISWYELQVPELRTLAIQRNRAVVEGIRKRLPPGAPAAAELLLHSVIAGATMQWAVDPDGELADHVLAQIAAILCLMFPEHDDFQLLQAHASAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 42 - protein, 380 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNRRRRR

SEQ ID NO: 43 - protein, 332 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRENLYFQSENLYFQSDYKDDDDKMAEPWGNELASAAARGDLEQLTSLLQNNVNVNAQNGFGRTALQVMKLGNPEIARRLLLRGANPDLKDRTGFAVIHDAARAGFLDTLQALPEFQADVNIEDNEGNLPLHLAAKEGHLRVVEFLVKHTASNVGHRNHKGDTACDLARLYGRNEVVSLMQANGAGGATNLQ

SEQ ID NO: 44 - DNA, 27 bp, synthetic construct
  source 1..27: mol_type = other DNA
cataccatgggagtgagcaagggcgag

SEQ ID NO: 45 - DNA, 30 bp, synthetic construct
  source 1..30: mol_type = other DNA
ggaagatctttacttgtacagctcgtccat

SEQ ID NO: 46 - DNA, 29 bp, synthetic construct
  source 1..29: mol_type = other DNA
cggggtacctcaactaaatgaccgtggtg

SEQ ID NO: 47 - DNA, 43 bp, synthetic construct
  source 1..43: mol_type = other DNA
gttaaagcttttcgaatctagactcgagcgtggcgaactggtc

SEQ ID NO: 48 - DNA, 35 bp, synthetic construct
  source 1..35: mol_type = other DNA
cagtctcgagcaaattctaaacaaaatacttccac

SEQ ID NO: 49 - DNA, 32 bp, synthetic construct
  source 1..32: mol_type = other DNA
cagtttcgaattaatttgtattgctttgacgg

SEQ ID NO: 50 - DNA, 34 bp, synthetic construct
  source 1..34: mol_type = other DNA
cagtctcgagactaacataacactatccacccag

SEQ ID NO: 51 - DNA, 28 bp, synthetic construct
  source 1..28: mol_type = other DNA
gttaaagctttcaggaggcattctgaag

SEQ ID NO: 52 - DNA, 28 bp, synthetic construct
  source 1..28: mol_type = other DNA
cagtctcgagcaggccatcaagtgtgtg

SEQ ID NO: 53 - DNA, 33 bp, synthetic construct
  source 1..33: mol_type = other DNA
cagtttcgaatcattttctcttcctcttcttca

SEQ ID NO: 54 - DNA, 24 bp, synthetic construct
  source 1..24: mol_type = other DNA
cagtctcgaggctgccatccggaa

SEQ ID NO: 55 - DNA, 28 bp, synthetic construct
  source 1..28: mol_type = other DNA
cagtttcgaatcacaagacaaggcaccc

SEQ ID NO: 56 - DNA, 34 bp, synthetic construct
  source 1..34: mol_type = other DNA
gttaaagcttggaggcattctgaagatacttatt

SEQ ID NO: 57 - DNA, 35 bp, synthetic construct
  source 1..35: mol_type = other DNA
cagtctcgagcaaatacagagcttctatcactcag

SEQ ID NO: 58 - DNA, 35 bp, synthetic construct
  source 1..35: mol_type = other DNA
gttaaagctttcaagatgtgattaatgaagaaatg

SEQ ID NO: 59 - DNA, 29 bp, synthetic construct
  source 1..29: mol_type = other DNA
cagtttcgaacccataaaaaagccctgtc

SEQ ID NO: 60 - DNA, 37 bp, synthetic construct
  source 1..37: mol_type = other DNA
gttaaagcttctactctatcatcaaacgataaaatgg

SEQ ID NO: 61 - DNA, 29 bp, synthetic construct
  source 1..29: mol_type = other DNA
cagtctcgagttcactcaagaaacgcaaa

SEQ ID NO: 62 - DNA, 32 bp, synthetic construct
  source 1..32: mol_type = other DNA
cagtttcgaattttctcttcctcttcttcacg

SEQ ID NO: 63 - DNA, 32 bp, synthetic construct
  source 1..32: mol_type = other DNA
cgtatctagaaaaatgatgaaaatggagactg

SEQ ID NO: 64 - DNA, 28 bp, synthetic construct
  source 1..28: mol_type = other DNA
gttaaagcttttagctggagacggtgac

SEQ ID NO: 65 - DNA, 29 bp, synthetic construct
  source 1..29: mol_type = other DNA
cagtctcgagttccagatcccagagtttg

SEQ ID NO: 66 - DNA, 24 bp, synthetic construct
  source 1..24: mol_type = other DNA
gttaaagctttcactgggaggggg

SEQ ID NO: 67 - DNA, 45 bp, synthetic construct
  source 1..45: mol_type = other DNA
cagtctcgagctcgagttatctactcatagaaactacttttgcag

SEQ ID NO: 68 - DNA, 29 bp, synthetic construct
  source 1..29: mol_type = other DNA
cgcggatcctcagtgtctctgcggcatta

SEQ ID NO: 69 - DNA, 43 bp, synthetic construct
  source 1..43: mol_type = other DNA
catttattcctcctagttagtcacagcaactgctgctcctttc

SEQ ID NO: 70 - DNA, 43 bp, synthetic construct
  source 1..43: mol_type = other DNA
gaaaggagcagcagttgctgtgactaactaggaggaataaatg

SEQ ID NO: 71 - DNA, 41 bp, synthetic construct
  source 1..41: mol_type = other DNA
cgattcacggattgctttctcattattccctccaggtacta

SEQ ID NO: 72 - DNA, 41 bp, synthetic construct
  source 1..41: mol_type = other DNA
tagtacctggagggaataatgagaaagcaatccgtgaatcg

SEQ ID NO: 73 - DNA, 30 bp, synthetic construct
  source 1..30: mol_type = other DNA
cgtatctagacggctttaagtgcgacattc

SEQ ID NO: 74 - DNA, 36 bp, synthetic construct
  source 1..36: mol_type = other DNA
cgtatctagactaaagtatgaggagagaaaattgaa

SEQ ID NO: 75 - DNA, 25 bp, synthetic construct
  source 1..25: mol_type = other DNA
gttaaagctttcagcttgccgtcgt

SEQ ID NO: 76 - DNA, 26 bp, synthetic construct
  source 1..26: mol_type = other DNA
cgtatctagagacccgttcctggtgc

SEQ ID NO: 77 - DNA, 26 bp, synthetic construct
  source 1..26: mol_type = other DNA
cgtatctagaccccccaagaagaagc

SEQ ID NO: 78 - DNA, 26 bp, synthetic construct
  source 1..26: mol_type = other DNA
gttaaagcttgctggagacggtgacc

SEQ ID NO: 79 - DNA, 30 bp, synthetic construct
  source 1..30: mol_type = other DNA
cgtatctagatcaggacgcttcggaggtag

SEQ ID NO: 80 - DNA, 30 bp, synthetic construct
  source 1..30: mol_type = other DNA
cgtatctagaatggactgtgaggtcaacaa

SEQ ID NO: 81 - DNA, 23 bp, synthetic construct
  source 1..23: mol_type = other DNA
cgtatctagaggcaaccgcagca

SEQ ID NO: 82 - DNA, 29 bp, synthetic construct
  source 1..29: mol_type = other DNA
gttaaagctttcagtccatcccatttctg

SEQ ID NO: 83 - DNA, 29 bp, synthetic construct
  source 1..29: mol_type = other DNA
cgtatctagatctggaatatccctggaca

SEQ ID NO: 84 - DNA, 30 bp, synthetic construct
  source 1..30: mol_type = other DNA
gttaaagcttgtctgtctcaatgccacagt

SEQ ID NO: 85 - DNA, 25 bp, synthetic construct
  source 1..25: mol_type = other DNA
cagtctcgagatgtccggggtggtg

SEQ ID NO: 86 - DNA, 28 bp, synthetic construct
  source 1..28: mol_type = other DNA
cagtttcgaatcactgcagcatgatgtc

SEQ ID NO: 87 - DNA, 31 bp, synthetic construct
  source 1..31: mol_type = other DNA
cagtctcgagagtggtgttgatgatgacatg

SEQ ID NO: 88 - DNA, 40 bp, synthetic construct
  source 1..40: mol_type = other DNA
cagtttcgaattagtgataaaaatagagttcttttgtgag

SEQ ID NO: 89 - DNA, 33 bp, synthetic construct
  source 1..33: mol_type = other DNA
cagtctcgagatgcacataactaatttgggatt

SEQ ID NO: 90 - DNA, 34 bp, synthetic construct
  source 1..34: mol_type = other DNA
cagtttcgaattatacaaatgacgaatacccttt

SEQ ID NO: 91 - DNA, 52 bp, synthetic construct
  source 1..52: mol_type = other DNA
gttaaagcttttacaccttgcgcttcttcttgggcgggctggagacggtgac

SEQ ID NO: 92 - DNA, 31 bp, synthetic construct
  source 1..31: mol_type = other DNA
cgtatctagaatggacttcaacaggaacttt

SEQ ID NO: 93 - DNA, 28 bp, synthetic construct
  source 1..28: mol_type = other DNA
cgtatctagaggacatagtccaccagcg

SEQ ID NO: 94 - DNA, 29 bp, synthetic construct
  source 1..29: mol_type = other DNA
gttaaagctttcagttggatccgaaaaac

SEQ ID NO: 95 - DNA, 34 bp, synthetic construct
  source 1..34: mol_type = other DNA
cgtatctagagaattaaaaaaaacactcatccca

SEQ ID NO: 96 - DNA, 29 bp, synthetic construct
  source 1..29: mol_type = other DNA
cgtatctagaccaaaggcaaaagcaaaaa

SEQ ID NO: 97 - DNA, 29 bp, synthetic construct
  source 1..29: mol_type = other DNA
gttaaagcttttagctagccatggcaagc

SEQ ID NO: 98 - DNA, 22 bp, synthetic construct
  source 1..22: mol_type = other DNA
cgtatctagaatgccccgcccc

SEQ ID NO: 99 - DNA, 31 bp, synthetic construct
  source 1..31: mol_type = other DNA
gttaaagcttctacccaccgtactcgtcaat

SEQ ID NO: 100 - DNA, 31 bp, synthetic construct
  source 1..31: mol_type = other DNA
cgtatctagaatgtctgacacgtccagagag

SEQ ID NO: 101 - DNA, 32 bp, synthetic construct
  source 1..32: mol_type = other DNA
gttaaagctttcatcttcttcgcaggaaaaag

SEQ ID NO: 102 - DNA, 30 bp, synthetic construct
  source 1..30: mol_type = other DNA
cgcggatccttatgggttctcacagcaaaa

SEQ ID NO: 103 - DNA, 43 bp, synthetic construct
  source 1..43: mol_type = other DNA
catttattcctcctagttagtcaaggcaacagccaatcaagag

SEQ ID NO: 104 - DNA, 43 bp, synthetic construct
  source 1..43: mol_type = other DNA
ctcttgattggctgttgccttgactaactaggaggaataaatg

SEQ ID NO: 105 - DNA, 41 bp, synthetic construct
  source 1..41: mol_type = other DNA
ttgattgcagtgacatggtgcattattccctccaggtacta

SEQ ID NO: 106 - DNA, 41 bp, synthetic construct
  source 1..41: mol_type = other DNA
tagtacctggagggaataatgcaccatgtcactgcaatcaa

SEQ ID NO: 107 - DNA, 30 bp, synthetic construct
  source 1..30: mol_type = other DNA
cgtatctagatagccgcagatgttggtatg

SEQ ID NO: 108 - DNA, 29 bp, synthetic construct
  source 1..29: mol_type = other DNA
cgtatctagagatcaagtccaactggtgg

SEQ ID NO: 109 - DNA, 29 bp, synthetic construct
  source 1..29: mol_type = other DNA
cagtctcgaggaaagcttgtttaaggggc

SEQ ID NO: 110 - DNA, 25 bp, synthetic construct
  source 1..25: mol_type = other DNA
cagtttcgaattagcgacggcgacg

SEQ ID NO: 111 - DNA, 31 bp, synthetic construct
  source 1..31: mol_type = other DNA
gttaaagcttttacttgtacagctcgtccat

SEQ ID NO: 112 - DNA, 25 bp, synthetic construct
  source 1..25: mol_type = other DNA
cgtatctagagtgagcaagggcgag

SEQ ID NO: 113 - DNA, 37 bp, synthetic construct
  source 1..37: mol_type = other DNA
cagtctcgagatggaagattataccaaaatagagaaa

SEQ ID NO: 114 - DNA, 36 bp, synthetic construct
  source 1..36: mol_type = other DNA
gttaaagcttctacatcttcttaatctgattgtcca

SEQ ID NO: 115 - DNA, 24 bp, synthetic construct
  source 1..24: mol_type = other DNA
cgtatctagaatggcgctgcagct

SEQ ID NO: 116 - DNA, 32 bp, synthetic construct
  source 1..32: mol_type = other DNA
gttaaagctttcagtcattgacaggaattttg

SEQ ID NO: 117 - DNA, 25 bp, synthetic construct
  source 1..25: mol_type = other DNA
cgtatctagaatggagccggcggcg

SEQ ID NO: 118 - DNA, 27 bp, synthetic construct
  source 1..27: mol_type = other DNA
gttaaagctttcaatcggggatgtctg

SEQ ID NO: 119 - DNA, 30 bp, synthetic construct
  source 1..30: mol_type = other DNA
cgtatctagaatgcgcgaggagaacaaggg

SEQ ID NO: 120 - DNA, 30 bp, synthetic construct
  source 1..30: mol_type = other DNA
gttaaagctttcagtcccctgtggctgtgc

SEQ ID NO: 121 - DNA, 24 bp, synthetic construct
  source 1..24: mol_type = other DNA
cgtatctagaatggccgagccttg

SEQ ID NO: 122 - DNA, 31 bp, synthetic construct
  source 1..31: mol_type = other DNA
gttaaagcttttattgaagatttgtggctcc

SEQ ID NO: 123 - DNA, 64 bp, synthetic construct
  source 1..64: mol_type = other DNA
cgtatctagagaaaatctgtattttcaaagtgaaaatctgtattttcaaagtatgccccgcccc

SEQ ID NO: 124 - DNA, 30 bp, synthetic construct
  source 1..30: mol_type = other DNA
gttaaagcttcccaccgtactcgtcaattc

SEQ ID NO: 125 - DNA, 66 bp, synthetic construct
  source 1..66: mol_type = other DNA
cgtatctagagaaaatctgtattttcaaagtgaaaatctgtattttcaaagtatggccgagccttg

SEQ ID NO: 126 - DNA, 29 bp, synthetic construct
  source 1..29: mol_type = other DNA
gttaaagcttttgaagatttgtggctccc

SEQ ID NO: 127 - DNA, 67 bp, synthetic construct
  source 1..67: mol_type = other DNA
cgtatctagagaaaatctgtattttcaaagtgaaaatctgtattttcaaagtgtgagcaagggcgag

SEQ ID NO: 128 - DNA, 91 bp, synthetic construct
  source 1..91: mol_type = other DNA
cgtatctagagaaaatctgtattttcaaagtgaaaatctgtattttcaaagtccgccgaaaaaaaaacgtaaagttgtgagcaagggcgag

SEQ ID NO: 129 - DNA, 55 bp, synthetic construct
  source 1..55: mol_type = other DNA
gttaaagcttttaaactttacgtttttttttcggcggcttgtacagctcgtccat

SEQ ID NO: 130 - DNA, 90 bp, synthetic construct
  source 1..90: mol_type = other DNA
cgtatctagagaaaatctgtattttcaaagtgaaaatctgtattttcaaagtgattataaagatgatgatgataaaatggccgagccttg

SEQ ID NO: 131 - DNA, 30 bp, synthetic construct
  source 1..30: mol_type = other DNA
cgtatctagaatgaccagttttgaagatgc

SEQ ID NO: 132 - DNA, 31 bp, synthetic construct
  source 1..31: mol_type = other DNA
gttaaagctttcatgactcattttcatccat

SEQ ID NO: 133 - DNA, 36 bp, synthetic construct
  source 1..36: mol_type = other DNA
cgtatctagaatgagtctcttaaactgtgagaacag

SEQ ID NO: 134 - DNA, 26 bp, synthetic construct
  source 1..26: mol_type = other DNA
gttaaagcttctacacccccgcatca

SEQ ID NO: 135 - protein, 405 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEVTNITLSTQHYRIHRSDVEPVKEKTTEKDIFAKSITAVRNSFISLSTSLSDRFSLHQQTDIPTTHFHRGNASEGRAVLTSKTVKDFMLQKLNSLDIKGNASKDPAYARQTCEAILSAVYSNNKDQCCKLLISKGVSITPFLKEIGEAAQNAGLPGEIKNGVFTPGGAGANPFVVPLIASASIKYPHMFINHNQQVSFKAYAEKIVMKEVTPLFNKGTMPTPQQFQLTIENIANKYLQNASKLGPEQKLISEEDLNSAVDHHHHHH

SEQ ID NO: 136 - protein, 435 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEFTQETQKMLIEKEIIPPLSYVDVASKIRESEVVKSSMQKIKTLCGVVYGNPDILEGKMPKMGIPVTNKNVEELEKFARQVGNFPSSCGKIVGFSFLGIKSEARAHAEENFLPLSHAIFSYAHNVKQAEKDILEAYFKEQERCAQSVETPSEEITNLLSFTQEQQKEILSNSPKLRTQVKAYSQKLHNRLSPNDLQAISERSHTKLAESLGTSVNQAEKIAQILTQTKDVVQILQQQEKLGLYQSIMKGDGRETAKVNMSAIKATQMTTKVTSLKAVEQIVRPPKVETAKVVSMSR

SEQ ID NO: 137 - protein, 354 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEQAIKCVVVGDGAVGKTCLLISYTTNAFPGEYIPTVFDNYSANVMVDGKPVNLGLWDTAGEEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKFEKLGPEQKLISEEDLNSAVDHHHHHH

SEQ ID NO: 138 - protein, 163 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRFEEIIHNIARHLAQIGDEMDH

SEQ ID NO: 139 - protein, 156 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEKKLSECLRRIGDELDS

SEQ ID NO: 140 - protein, 20 aa, Salmonella enterica
MPYTSVSTYARALSGNKLPH

SEQ ID NO: 141 - protein, 210 aa, Salmonella enterica
MPYTSVSTYARALSGNKLPHVAAGDYENKLSTKIMKGILYVLTAGLAYGFTRVIEHYCNVTPKVAEFCANAGNIHNHLADAVRDGLFTIDVELSDGRMLTFEQLSLIAEGKPIVRISDGEHTVEVEGTFEEICMRLEEGFFEAPAYYDYDIDEKYKTVRERMAAYNALPQALGAIPCLEYYIARASNMQEAKAQWAADIKARYHNYLDNY

SEQ ID NO: 142 - protein, 81 aa, Salmonella enterica
VTKITLSPQNFRIQKQETTLLKEKSTEKNSLAKSILAVKNHFIELRSKLSERFISHKNTESSATHFHRGSASEGRAVLTNK

SEQ ID NO: 143 - protein, 105 aa, Salmonella enterica
VTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPA

SEQ ID NO: 144 - protein, 158 aa, synthetic construct
MPYTSVSTYARALSGNKLPHGTGSQASRSFNQGRIEPDSESQEEIIHNIARHLAQIGDEMDHNIQPTLVRQLAAQFMNGSLSEEDKRNCLAKALDEVKTAFPRDMENDKAMLIMTMLLAKKVASHAPSLLRDVFHTTVNFINQNLFSYVRNLVRNEMD

SEQ ID NO: 145 - protein, 348 aa, synthetic construct
MPYTSVSTYARALSGNKLPHVAAGDYENKLSTKIMKGILYVLTAGLAYGFTRVIEHYCNVTPKVAEFCANAGNIHNHLADAVRDGLFTIDVELSDGRMLTFEQLSLIAEGKPIVRISDGEHTVEVEGTFEEICMRLEEGFFEAPAYYDYDIDEKYKTVRERMAAYNALPQALGAIPCLEYYIARASNMQEAKAQWAADIKARYHNYLDNYGTGSQASRSFNQGRIEPDSESQEEIIHNIARHLAQIGDEMDHNIQPTLVRQLAAQFMNGSLSEEDKRNCLAKALDEVKTAFPRDMENDKAMLIMTMLLAKKVASHAPSLLRDVFHTTVNFINQNLFSYVRNLVRNEMD

SEQ ID NO: 146 - protein, 219 aa, synthetic construct
VTKITLSPQNFRIQKQETTLLKEKSTEKNSLAKSILAVKNHFIELRSKLSERFISHKNTESSATHFHRGSASEGRAVLTNKGTGSQASRSFNQGRIEPDSESQEEIIHNIARHLAQIGDEMDHNIQPTLVRQLAAQFMNGSLSEEDKRNCLAKALDEVKTAFPRDMENDKAMLIMTMLLAKKVASHAPSLLRDVFHTTVNFINQNLFSYVRNLVRNEMD

SEQ ID NO: 147 - protein, 243 aa, synthetic construct
VTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPAGTGSQASRSFNQGRIEPDSESQEEIIHNIARHLAQIGDEMDHNIQPTLVRQLAAQFMNGSLSEEDKRNCLAKALDEVKTAFPRDMENDKAMLIMTMLLAKKVASHAPSLLRDVFHTTVNFINQNLFSYVRNLVRNEMD

SEQ ID NO: 148 - DNA, 82 bp, synthetic construct
  source 1..82: mol_type = other DNA
ttactattcgaagaaattattcataatattgcccgccatctggcccaaattggtgatgaaatggatcattaagcttggagta

SEQ ID NO: 149 - DNA, 82 bp, synthetic construct
  source 1..82: mol_type = other DNA
tactccaagcttaatgatccatttcatcaccaatttgggccagatggcgggcaatattatgaataatttcttcgaatagtaa

SEQ ID NO: 150 - DNA, 73 bp, synthetic construct
  source 1..73: mol_type = other DNA
ttactactcgagaaaaaactgagcgaatgtctgcgccgcattggtgatgaactggatagctaagcttggagta

SEQ ID NO: 151 - DNA, 73 bp, synthetic construct
  source 1..73: mol_type = other DNA
tactccaagcttagctatccagttcatcaccaatgcggcgcagacattcgctcagttttttctcgagtagtaa

SEQ ID NO: 152 - DNA, 34 bp, synthetic construct
  source 1..34: mol_type = other DNA
catgccatggatttatggtcatagatatgacctc

SEQ ID NO: 153 - DNA, 35 bp, synthetic construct
  source 1..35: mol_type = other DNA
cggggtaccatgaggtagcttatttcctgataaag

SEQ ID NO: 154 - DNA, 35 bp, synthetic construct
  source 1..35: mol_type = other DNA
cggggtaccataattgtccaaatagttatggtagc

SEQ ID NO: 155 - DNA, 24 bp, synthetic construct
  source 1..24: mol_type = other DNA
catgccatggcggcaaggctcctc

SEQ ID NO: 156 - DNA, 29 bp, synthetic construct
  source 1..29: mol_type = other DNA
cggggtacctttatttgtcaacactgccc

SEQ ID NO: 157 - DNA, 26 bp, synthetic construct
  source 1..26: mol_type = other DNA
cggggtacctgcggggtctttactcg

SEQ ID NO: 158 - protein, 164 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEGAIDDAAREGFLDTLVVLHRAGAR

SEQ ID NO: 159 - protein, 164 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEGAIDYGRKKRRQRRRGPVKRRLFG

SEQ ID NO: 160 - protein, 164 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEGAIDKRRQTSMTAFYHSKRRLIFS

SEQ ID NO: 161 - protein, 160 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEGAIDTSMTAFYHSKRRLIFS

SEQ ID NO: 162 - protein, 161 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEGAIDACRRLFGPVDSEQLSRD

SEQ ID NO: 163 - protein, 153 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEGAIDWELVVLGKL

SEQ ID NO: 164 - protein, 397 aa, synthetic construct
MPYTSVSTYARALSGNKLPHVAAGDYENKLSTKIMKGILYVLTAGLAYGFTRVIEHYCNVTPKVAEFCANAGNIHNHLADAVRDGLFTIDVELSDGRMLTFEQLSLIAEGKPIVRISDGEHTVEVEGTFEEICMRLEEGFFEAPAYYDYDIDEKYKTVRERMAAYNALPQALGAIPCLEYYIARASNMQEAKAQWAADIKARYHNYLDNYGTIWEFMEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPDKLGPEQKLISEEDLNSAVDHHHHHH

SEQ ID NO: 165 - protein, 292 aa, synthetic construct
VTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPAGTIWEFMEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPDKLGPEQKLISEEDLNSAVDHHHHHH

SEQ ID NO: 166 - protein, 409 aa, synthetic construct
MPYTSVSTYARALSGNKLPHVAAGDYENKLSTKIMKGILYVLTAGLAYGFTRVIEHYCNVTPKVAEFCANAGNIHNHLADAVRDGLFTIDVELSDGRMLTFEQLSLIAEGKPIVRISDGEHTVEVEGTFEEICMRLEEGFFEAPAYYDYDIDEKYKTVRERMAAYNALPQALGAIPCLEYYIARASNMQEAKAQWAADIKARYHNYLDNYGTIWEFMAEPWGNELASAAARGDLEQLTSLLQNNVNVNAQNGFGRTALQVMKLGNPEIARRLLLRGANPDLKDRTGFAVIHDAARAGFLDTLQTLLEFQADVNIEDNEGNLPLHLAAKEGHLRVVEFLVKHTASNVGHRNHKGDTACDLARLYGRNEVVSLMQANGAGGATNLQKLGPEQKLISEEDLNSAVDHHHHHH

SEQ ID NO: 167 - protein, 304 aa, synthetic construct
VTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPAGTIWEFMAEPWGNELASAAARGDLEQLTSLLQNNVNVNAQNGFGRTALQVMKLGNPEIARRLLLRGANPDLKDRTGFAVIHDAARAGFLDTLQTLLEFQADVNIEDNEGNLPLHLAAKEGHLRVVEFLVKHTASNVGHRNHKGDTACDLARLYGRNEVVSLMQANGAGGATNLQKLGPEQKLISEEDLNSAVDHHHHHH

SEQ ID NO: 168 - protein, 446 aa, synthetic construct
MPYTSVSTYARALSGNKLPHVAAGDYENKLSTKIMKGILYVLTAGLAYGFTRVIEHYCNVTPKVAEFCANAGNIHNHLADAVRDGLFTIDVELSDGRMLTFEQLSLIAEGKPIVRISDGEHTVEVEGTFEEICMRLEEGFFEAPAYYDYDIDEKYKTVRERMAAYNALPQALGAIPCLEYYIARASNMQEAKAQWAADIKARYHNYLDNYGTIWEFMALQLSREQGITLRGSAEIVAEFFSFGINSILYQRGIYPSETFTRVQKYGLTLLVTTDLELIKYLNNVVEQLKDWLYKCSVQKLVVVISNIESGEVLERWQFDIECDKTAKDDSAPREKSQKAIQDEIRSVIRQITATVTFLPLLEVSCSFDLLIYTDKDLVVPEKWEESGPQFITNSEEVRLRSFTTTIHKVNSMVAYKIPVNDKLGPEQKLISEEDLNSAVDHHHHHH

SEQ ID NO: 169 - protein, 341 aa, synthetic construct
VTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPAGTIWEFMALQLSREQGITLRGSAEIVAEFFSFGINSILYQRGIYPSETFTRVQKYGLTLLVTTDLELIKYLNNVVEQLKDWLYKCSVQKLVVVISNIESGEVLERWQFDIECDKTAKDDSAPREKSQKAIQDEIRSVIRQITATVTFLPLLEVSCSFDLLIYTDKDLVVPEKWEESGPQFITNSEEVRLRSFTTTIHKVNSMVAYKIPVNDKLGPEQKLISEEDLNSAVDHHHHHH

SEQ ID NO: 170 - protein, 538 aa, synthetic construct
MPYTSVSTYARALSGNKLPHVAAGDYENKLSTKIMKGILYVLTAGLAYGFTRVIEHYCNVTPKVAEFCANAGNIHNHLADAVRDGLFTIDVELSDGRMLTFEQLSLIAEGKPIVRISDGEHTVEVEGTFEEICMRLEEGFFEAPAYYDYDIDEKYKTVRERMAAYNALPQALGAIPCLEYYIARASNMQEAKAQWAADIKARYHNYLDNYGTIWEFMEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKMKLGPEQKLISEEDLNSAVDHHHHHH

SEQ ID NO: 171 - protein, 433 aa, synthetic construct
VTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPAGTIWEFMEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKMKLGPEQKLISEEDLNSAVDHHHHHH

SEQ ID NO: 172 - DNA, 95 bp, synthetic construct
  source 1..95: mol_type = other DNA
catgctcgagggtgccatcgatgatgccgcccgcgaaggttttctggataccctggtggtgctgcatcgcgccggtgcccgctaattcgaacatg

SEQ ID NO: 173 - DNA, 95 bp, synthetic construct
  source 1..95: mol_type = other DNA
catgttcgaattagcgggcaccggcgcgatgcagcaccaccagggtatccagaaaaccttcgcgggcggcatcatcgatggcaccctcgagcatg

SEQ ID NO: 174 - DNA, 95 bp, synthetic construct
  source 1..95: mol_type = other DNA
catgctcgagggtgccatcgattatggtcgcaaaaaacgccgccaacgccgccgcggtccggtgaaacgccgcctgtttggttaattcgaacatg

SEQ ID NO: 175 - DNA, 95 bp, synthetic construct
  source 1..95: mol_type = other DNA
catgttcgaattaaccaaacaggcggcgtttcaccggaccgcggcggcgttggcggcgttttttgcgaccataatcgatggcaccctcgagcatg

SEQ ID NO: 176 - DNA, 95 bp, synthetic construct
  source 1..95: mol_type = other DNA
catgctcgagggtgccatcgataaacgccgccaaaccagcatgaccgccttttatcatagcaaacgccgcctgatttttagctaattcgaacatg

SEQ ID NO: 177 - DNA, 95 bp, synthetic construct
  source 1..95: mol_type = other DNA
catgttcgaattagctaaaaatcaggcggcgtttgctatgataaaaggcggtcatgctggtttggcggcgtttatcgatggcaccctcgagcatg

SEQ ID NO: 178 - DNA, 83 bp, synthetic construct
  source 1..83: mol_type = other DNA
catgctcgagggtgccatcgataccagcatgaccgccttttatcatagcaaacgccgcctgatttttagctaattcgaacatg

SEQ ID NO: 179 - DNA, 83 bp, synthetic construct
  source 1..83: mol_type = other DNA
catgttcgaattagctaaaaatcaggcggcgtttgctatgataaaaggcggtcatgctggtatcgatggcaccctcgagcatg

SEQ ID NO: 180 - DNA, 86 bp, synthetic construct
  source 1..86: mol_type = other DNA
catgctcgagggtgccatcgatgcctgtcgccgcctgtttggtccggtggatagcgaacaactgagccgcgattaattcgaacatg

SEQ ID NO: 181 - DNA, 86 bp, synthetic construct
  source 1..86: mol_type = other DNA
catgttcgaattaatcgcggctcagttgttcgctatccaccggaccaaacaggcggcgacaggcatcgatggcaccctcgagcatg

SEQ ID NO: 182 - DNA, 62 bp, synthetic construct
  source 1..62: mol_type = other DNA
catgctcgagggtgccatcgattgggaactggtggtgctgggtaaactgtaattcgaacatg

SEQ ID NO: 183 - DNA, 62 bp, synthetic construct
  source 1..62: mol_type = other DNA
catgttcgaattacagtttacccagcaccaccagttcccaatcgatggcaccctcgagcatg

SEQ ID NO: 184 - DNA, 27 bp, synthetic construct
  source 1..27: mol_type = other DNA
gacatggaattcatggagccggcggcg

SEQ ID NO: 185 - DNA, 28 bp, synthetic construct
  source 1..28: mol_type = other DNA
catgaagcttatcggggatgtctgaggg

SEQ ID NO: 186 - DNA, 29 bp, synthetic construct
  source 1..29: mol_type = other DNA
gacatggaattcatggccgagccttgggg

SEQ ID NO: 187 - DNA, 51 bp, synthetic construct
  source 1..51: mol_type = other DNA
gttaacatcagcttgaaactccagcaaagtctgtaaagtgtccaggaaacc

SEQ ID NO: 188 - DNA, 51 bp, synthetic construct
  source 1..51: mol_type = other DNA
ggtttcctggacactttacagactttgctggagtttcaagctgatgttaac

SEQ ID NO: 189 - DNA, 30 bp, synthetic construct
  source 1..30: mol_type = other DNA
catgaagcttttgaagatttgtggctcccc

SEQ ID NO: 190 - DNA, 30 bp, synthetic construct
  source 1..30: mol_type = other DNA
gacatggaattcatggcgctgcagctctcc

SEQ ID NO: 191 - DNA, 33 bp, synthetic construct
  source 1..33: mol_type = other DNA
catgaagcttgtcattgacaggaattttgtagg

SEQ ID NO: 192 - DNA, 38 bp, synthetic construct
  source 1..38: mol_type = other DNA
gacatggaattcatggaagattataccaaaatagagaa

SEQ ID NO: 193 - DNA, 34 bp, synthetic construct
  source 1..34: mol_type = other DNA
catgaagcttcatcttcttaatctgattgtccaa

SEQ ID NO: 194 - protein, 276 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEGSQASRSFNQGRIEPDSESQEEIIHNIARHLAQIGDEMDHNIQPTLVRQLAAQFMNGSLSEEDKRNCLAKALDEVKTAFPRDMENDKAMLIMTMLLAKKVASHAPSLLRDVFHTTVNFINQNLFSYVRNLVRNEMD

SEQ ID NO: 195 - protein, 245 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMQIFVKTLTGKTITLEVEPSDTIENVKAKIQDKEGIPPDQQRLIFAGKQLEDGRTLSDYNIQKESTLHLVLRLRGGFEASKLGPEQKLISEEDLNSAVDHHHHHH

SEQ ID NO: 196 - protein, 419 aa, synthetic construct
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLEMQIFVKTLTGKTITLEVEPSDTIENVKAKIQDKEGIPPDQQRLIFAGKQLEDGRTLSDYNIQKESTLHLVLRLRGGFEDYKDDDDKMAEPWGNELASAAARGDLEQLTSLLQNNVNVNAQNGFGRTALQVMKLGNPEIARRLLLRGANPDLKDRTGFAVIHDAARAGFLDTLQALPEFQADVNIEDNEGNLPLHLAAKEGHLRVVEFLVKHTASNVGHRNHKGDTACDLARLYGRNEVVSLMQANGAGGATNLQKLGPEQKLISEEDLNSAVDHHHHHH

SEQ ID NO: 197 - DNA, 30 bp, synthetic construct
  source 1..30: mol_type = other DNA
cagtctcgagatgcagatcttcgtcaagac

SEQ ID NO: 198 - DNA, 43 bp, synthetic construct
  source 1..43: mol_type = other DNA
gttaaagcttgctagcttcgaaaccaccacgtagacgtaagac

SEQ ID NO: 199 - DNA, 48 bp, synthetic construct
  source 1..48: mol_type = other DNA
cagtttcgaagattataaagatgatgatgataaaatggccgagccttg

SEQ ID NO: 200 - DNA, 138 bp, synthetic construct
  source 1..138: mol_type = other DNA
gttcgccacgctcgagtctagattcgaaaagcttgggcccgaacaaaaactcatctcagaagaggatctgaatagcgccgtcgaccatcatcatcatcatcattgagtttaaacggtctccagcttggctgttttggc

SEQ ID NO: 201 - protein, 8 aa, synthetic construct
PPKKKRKV

(19) United States
(12) Patent Application Publication
(10) Pub. No.: US 2024/0401065 A1
(43) Pub. Date: Dec. 5, 2024

(54) Bacteria-Based Protein Delivery

(71) Applicant: Universitaet Basel, Basel (CH)

(72) Inventors: Cécile Arrieumerlou, Basel (CH); Simon Ittig, Basel (CH)

(21) Appl. No.: 18/806,599

(22) Filed: Aug. 15, 2024

Related U.S. Application Data
(63) Continuation of application No. 17/111,332, filed on Dec. 3, 2020, now Pat. No. 12,091,670.
Application No. 17/111,332 is a continuation of application No. 15/311,424, filed on Nov. 15, 2016, now Pat. No. 10,889,823.

(30) Foreign Application Priority Data
May 21, 2014 (EP) 14169335.8

(51) Int. Cl. C12N 15/74; A61K 48/00; C07K 14/195; C07K 14/24; C12N 15/70; C12Q 1/02

(52) U.S. Cl. CPC C12N 15/74; C07K 14/195; C07K 14/24; C12N 15/70; C12Q 1/025; A61K 48/00; C07K 2319/035; C07K 2319/50; G01N 2333/24; G01N 2333/245; G01N 2333/255; G01N 2500/10

(57) ABSTRACT

The present invention relates to recombinant Gram-negative bacterial strains and the use thereof for delivery of heterologous proteins into eukaryotic cells.

### CROSS-REFERENCE TO RELATED APPLICATIONS

This application is a continuation of U.S. application Ser. No. 17/111,332, filed Dec. 3, 2020, which is a continuation of U.S. application Ser. No. 15/311,424, filed on Nov. 15, 2016, which application is a 371 of International Application No. PCT/EP2015/061086, filed on May 20, 2015, which application claims priority from European Application No. 14169335.8, filed May 21, 2014, the content of which is incorporated herein by reference.

### INCORPORATION BY REFERENCE OF SEQUENCE LISTING PROVIDED AS A SEQUENCE LISTING XML FILE

A Sequence Listing is provided herewith as a Sequence Listing XML, “LATS-003CON2_SEQ_LIST.xml”, created on Aug. 14, 2024, and having a size of 202,930 bytes. The contents of the Sequence Listing XML are incorporated herein by reference in its entirety.

### THE FIELD OF THE INVENTION

The present invention relates to recombinant Gram-negative bacterial strains and the use thereof for delivery of heterologous proteins into eukaryotic cells.

### BACKGROUND OF THE INVENTION

Transient transfection techniques have been applied in cell biological research over many years to address protein functions. These methods generally result in a massive overrepresentation of the protein under study, which might lead to oversimplified models of signalling [1]. For proteins controlling short-lived signalling processes, the protein of interest is present for far longer as the signalling event it controls [2]. Even more, DNA transfection based transient over-expression leads to a heterogenous and unsynchronized cell population, which complicates functional studies and hampers-omics approaches. Besides this, the upscaling of such assays to a larger scale is very expensive. Some of the above mentioned points are covered by existing techniques as microinjection or proteo-fection of purified proteins, the inducible translocation strategy to rapidly target plasmid born small GTPases to the cell membrane [2] or the addition of purified proteins fused to cell-permeable bacterial toxins [3]. But these techniques are all time-consuming and cumbersome and to our knowledge none fulfils all mentioned criteria.

Bacteria have evolved different mechanisms to directly inject proteins into target cells [4]. The type III secretion system (T3SS) used by bacteria like Yersinia, Shigella and Salmonella [5] functions like a nano-syringe that injects so-called bacterial effector proteins into host cells. Bacterial proteins to be secreted via the T3SS, called effectors, harbour a short N-terminal secretion signal [6]. Inside bacteria, some effectors are bound by chaperones. Chaperones might mask toxic domains [7], they contribute to exposition of the secretion signal [8, 9] and keep the substrates in a secretion-competent conformation [10], therefore facilitating secretion. Upon induction of secretion, an ATPase adjacent to the T3SS removes the chaperones and the effectors travel unfolded or only partially folded through the needle [10], and refold once in the host cytoplasm.

T3S has been exploited to deliver hybrid peptides and proteins into target cells. Heterologous bacterial T3SS effectors have been delivered in case the bacterium under study is hardly

accessible by genetics (like Chlamydia trachomatis; [12]). Often reporter proteins were fused to possible T3SS secretion signals as to study requirements for T3SS dependent protein delivery, such as the Bordetella pertussis adenylate cyclase [13], murine DHFR [10] or a phosphorylatable tag [14]. Peptide delivery was mainly conducted with the aim of vaccination. This includes viral epitopes [15, 16], bacterial epitopes (listeriolysin O, [17]) as well as peptides representing epitopes of human cancer cells [18]. In few cases functional eukaryotic proteins have been delivered to modulate the host cell, as done with nanobodies [19], nuclear proteins (Cre-recombinase, MyoD) [20, 21] or I110 and IL1ra [22]. None of the above-mentioned systems allows single-protein delivery as in each case one or multiple endogenous effector proteins are still encoded. Furthermore, the vectors used have not been designed in a way allowing simple cloning of other DNA fragments encoding proteins of choice, hindering broad application of the system.

Therefore, a cheap and simple method allowing scalable, rapid, synchronized, homogenous and tuneable delivery of a protein of interest at physiological concentrations would be of great benefit for many cell biologists.

### SUMMARY OF THE INVENTION

The present invention relates generally to recombinant Gram-negative bacterial strains and the use thereof for delivery of heterologous proteins into eukaryotic cells. The present invention provides Gram-negative bacterial strains and the use thereof, which allows the translocation of various type III effectors, but also of type IV effectors, of viral proteins and most importantly of functional eukaryotic proteins. Means for fluorescent tracking of delivery, for relocalization to the nucleus and notably for removal of the bacterial appendage after delivery to the host cell are provided. This allows for the first time delivery of almost native proteins into eukaryotic cells using only a T3SS. The presented T3SS based system results in scalable, rapid, synchronized, homogenous and tunable delivery of a protein of interest. The delivery system of the present invention is suitable to inject eukaryotic proteins in living animals and can be used for therapeutic purposes.

In a first aspect the present invention relates to a recombinant Gram-negative bacterial strain selected from the group consisting of the genera Yersinia, Escherichia, Salmonella and Pseudomonas, wherein said Gram-negative bacterial strain is transformed with a vector which comprises in the 5′ to 3′ direction:
a promoter;
a first DNA sequence encoding a delivery signal from a bacterial T3SS effector protein, operably linked to said promoter; and
a second DNA sequence encoding a heterologous protein fused in frame to the 3′end of said first DNA sequence, wherein the heterologous protein is selected from the group consisting of proteins involved in apoptosis or apoptosis regulation.

In a further aspect, the present invention relates to a recombinant Gram-negative bacterial strain transformed with a vector, which comprises in the 5′ to 3′ direction:
a promoter;
a first DNA sequence encoding a delivery signal from a bacterial T3SS effector protein, operably linked to said promoter;
a second DNA sequence encoding a heterologous protein fused in frame to the 3′end of said first DNA sequence; and
a third DNA sequence encoding a protease cleavage site, wherein the third DNA sequence is located between the 3′end of said first DNA sequence and the 5′end of said second DNA sequence.

In a further aspect the present invention relates to a recombinant Gram-negative bacterial strain, wherein the recombinant Gram-negative bacterial strain is a Yersinia strain and wherein said Yersinia strain is wild type or deficient in the production of at least one T3SS effector protein and is transformed with a vector which comprises in the 5′ to 3′ direction:
a promoter;
a first DNA sequence encoding a delivery signal from a bacterial T3SS effector protein wherein the delivery signal from the bacterial T3SS effector protein comprises the N-terminal 138 amino acids of the Y. enterocolitica YopE effector protein, operably linked to said promoter; and
a second DNA sequence encoding a heterologous protein fused in frame to the 3′end of said first DNA sequence.

In a further aspect the present invention relates to a recombinant Gram-negative bacterial strain, wherein the recombinant Gram-negative bacterial strain is a Salmonella strain and wherein said Salmonella strain is wild type or deficient in the production of at least one T3SS effector protein and is transformed with a vector which comprises in the 5′ to 3′ direction:
a promoter;
a first DNA sequence encoding a delivery signal from a bacterial T3SS effector protein wherein the delivery signal from the bacterial T3SS effector protein comprises the S. enterica SteA effector protein or the N-terminal 81 or 105 amino acids of the S. enterica SopE effector protein, operably linked to said promoter; and
a second DNA sequence encoding a heterologous protein fused in frame to the 3′end of said first DNA sequence.

In a further aspect the present invention relates to a vector which comprises in the 5′ to 3′ direction:
a promoter;
a first DNA sequence encoding a delivery signal from a bacterial T3SS effector protein, operably linked to said promoter;
a second DNA sequence encoding a heterologous protein fused in frame to the 3′end of said first DNA sequence; and
a third DNA sequence encoding a protease cleavage site, wherein the third DNA sequence is located between the 3′end of said first DNA sequence and the 5′end of said second DNA sequence.

The present invention further relates to a method for delivering a heterologous protein into a eukaryotic cell comprising the following steps:
i) culturing a Gram-negative bacterial strain; and
ii) contacting a eukaryotic cell with the Gram-negative bacterial strain of i) wherein a fusion protein which comprises a delivery signal from a bacterial T3SS effector protein and the heterologous protein is expressed by the Gram-negative bacterial strain and is translocated into the eukaryotic cell.

The present invention further relates to a method for delivering a heterologous protein into a eukaryotic cell comprising the following steps:
i) culturing a Gram-negative bacterial strain;
ii) contacting a eukaryotic cell with the Gram-negative bacterial strain of i) wherein a fusion protein which comprises a delivery signal from a bacterial T3SS effector protein and the heterologous protein is expressed by the Gram-negative bacterial strain and is translocated into the eukaryotic cell; and
iii) cleaving the fusion protein so that the heterologous protein is cleaved from the delivery signal from the bacterial T3SS effector protein.

The present invention further relates to a method of purifying a heterologous protein comprising culturing a Gram-negative bacterial strain so that a fusion protein which comprises a delivery signal from a bacterial T3SS effector protein and the heterologous protein is expressed and secreted into the supernatant of the culture.

In a further aspect the present invention relates to a library of Gram-negative bacterial strains, wherein the heterologous protein encoded by the second DNA sequence of the expression vector of the Gram-negative bacterial strains is a human or murine protein and, wherein each human or murine expressed by a Gram-negative bacterial strain is different in amino acid sequence.

### BRIEF DESCRIPTION OF THE FIGURES

FIG. 1: Characterization of T3SS protein delivery. (A) Schematic representation of T3SS dependent protein secretion into the surrounding medium (in-vitro secretion) (left side) or into eukaryotic cells (right side). I: shows the type 3 secretion system. II indicates proteins secreted into the surrounding medium, III proteins translocated through the membrane into the cytosol of eukaryotic cells (VII). VI shows a stretch of the two bacterial membranes in which the T3SS is inserted and the bacterial cytosol underneath. IV is a fusion protein attached to the YopE₁₋₁₃₈ N-terminal fragment (V) (B) In-vitro secretion of I: Y. enterocolitica E40 wild type, II: Y. enterocolitica ΔHOPEMT asd or III: Y. enterocolitica ΔHOPEMT asd+pBadSi_2 as revealed by Western blotting on total bacterial lysates (IV) and precipitated culture supernatants (V) using an anti-YopE antibody.

FIG. 2: Characterization of T3SS protein delivery into epithelial cells. (A) Anti-Myc immunofluorescence staining on HeLa cells infected at an MOI of 100 for 1 h with I: Y. enterocolitica ΔHOPEMT asd or II: Y. enterocolitica ΔHOPEMT asd+pBad_Si2. (B) Quantification of anti-Myc immunofluorescence staining intensity from (A) within HeLa cells. Data were combined from n=20 sites, error bars indicated are standard error of the mean. I: uninfected II: Y. enterocolitica ΔHOPEMT asd or III: Y. enterocolitica ΔHOPEMT asd+pBad_Si2. Y-axis indicates anti-Myc staining intensity [arbitrary unit], x-axis indicates time of infection in minutes (C) Quantification of Anti-Myc immunofluorescence staining intensity within cells. HeLa cells were infected for 1 h with Y. enterocolitica ΔHOPEMT asd+pBad_Si2 at an MOI indicated on the x-axis. Data were combined from n=20 sites, error bars indicated are standard error of the mean. Y-axis indicates anti-Myc staining intensity [a.u.].

FIG. 3: Modifications of the T3SS based protein delivery allow nuclear localization of a YopE₁₋₁₃₈ fusion protein (EGFP). EGFP signal in Hela cells infected with I: Y. enterocolitica ΔHOPEMT asd or II: Y. enterocolitica ΔHOPEMT asd ΔyopB carrying the plasmids III: +YopE₁₋₁₃₈-EGFP or IV: +YopE₁₋₁₃₈-EGFP-NLS at an MOI of 100. EGFP signal is shown in “a”, for localization comparison nuclei were stained in “b”.

FIG. 4: Modifications of the T3SS based protein delivery allow removal of the YopE₁₋₁₃₈ appendage. HeLa cells are infected with two different Y. enterocolitica strains at the same time, which is reached by simple mixing of the two bacterial suspensions. One strain is delivering the TEV protease fused to YopE₁₋₁₃₈, while the other strain delivers a protein of interest fused to YopE₁₋₁₃₈ with a linker containing a double TEV protease cleavage site. After protein delivery into the eukaryotic cell, the TEV protease will cleave the YopE₁₋₁₃₈ appendage from the protein of interest (A) Digitonin lysed HeLa cells uninfected (II) or after infection (MOI of 100) for 2 h with I: Y. enterocolitica ΔHOPEMT asd and III: +pBadSi_2, IV: +YopE₁₋₁₃₈-2x TEV cleavage site-Flag-INK4C, V: +YopE₁₋₁₃₈-2x TEV cleavage site-Flag-INK4C and further overnight treatment with purified TEV protease and VI: +YopE₁₋₁₃₈-2x TEV cleavage site-Flag-INK4C and a second strain +YopE₁₋₁₃₈-TEV were analyzed by Western blotting anti-INK4C (shown in “a”) for the presence of YopE₁₋₁₃₈-2x TEV cleavage site-Flag-INK4C or its cleaved form Flag-INK4C. As a loading control western blotting anti-Actin was performed (shown in “b”). In one case (V) the lysed cells were incubated overnight with purified TEV protease. (B) Actin normalized quantification of anti-INK4C staining intensity (shown as [a.u.] on the y-axis) from (A) at the size of full length YopE₁₋₁₃₈-2x TEV cleavage site-Flag-INK4C, where sample IV is set to 100%. I: Y. enterocolitica ΔHOPEMT asd and IV: +YopE₁₋₁₃₈-2x TEV cleavage site-Flag-INK4C, V: +YopE₁₋₁₃₈-2x TEV cleavage site-Flag-INK4C and further overnight treatment with purified TEV protease and VI: +YopE₁₋₁₃₈-2x TEV cleavage site-Flag-INK4C and a second strain+YopE₁₋₁₃₈-TEV. Data were combined from n=2 independent experiments, error bars indicated are standard error of the mean (C) Digitonin lysed HeLa cells uninfected (II) or after infection (MOI of 100) for 2 h with I: Y. enterocolitica ΔHOPEMT asd and III: +pBadSi_2, IV: +YopE₁₋₁₃₈-2x TEV cleavage site-ET1-Myc, V: +YopE₁₋₁₃₈-2x TEV cleavage site—ETI-Myc and further overnight treatment with purified TEV protease and VI: +YopE₁₋₁₃₈-2x TEV cleavage site-ET1-Myc and a second strain+YopE₁₋₁₃₈-TEV were analyzed by Western blotting anti-Myc (shown in “a”) for the presence of YopE₁₋₁₃₈-2x TEV cleavage site—ET1-Myc or its cleaved form ET1-Myc. As a loading control western blotting anti-Actin was performed (shown in “b”) In one case (V) the lysed cells were incubated overnight with purified TEV protease.

FIG. 5: Delivery of bacterial effector proteins into eukaryotic cells (A) HeLa cells were infected with I: Y. enterocolitica ΔHOPEMT asd carrying II: pBad_Si2 or III: YopE₁₋₁₃₈-SopE at an MOI of 100 for the time indicated above the images (2, 10 or 60 minutes). After fixation cells were stained for the actin cytoskeleton (B) HeLa cells were left uninfected (II) or infected with I: Y. enterocolitica ΔHOPEMT asd carrying III: YopE₁₋₁₃₈-SopE-Myc and in some cases coinfected with IV: YopE₁₋₁₃₈-SptP at the MOI indicated below the strain (MOI 50; MOI50:MOI50 or MOI50:MOI100) for 1 h. After fixation cells were stained for the actin cytoskeleton (shown in “a”) and the presence of the YopE₁₋₁₃₈-SopE-Myc fusion protein was followed via staining anti-Myc (shown in “b”).

FIG. 6: Delivery of bacterial effector proteins into eukaryotic cells (A) Phospho-p38 (“a”), total p38 (“b”) and actin (“c”) western blot analysis on HeLa cells left untreated (II) or infected for 75 min with I: Y. enterocolitica ΔHOPEMT asd carrying III: pBad_Si2 or IV: YopE₁₋₁₃₈-OspF at an MOI of 100. Cells were stimulated with TNFα for the last 30 min of the infection as indicated (+stands for addition of TNFα, —represent no treatment with TNFα) (B) Phospho-Akt T308 (“a”) and S473 (“b”) and actin (“c”) western blot analysis on HeLa cells left untreated (II) or infected for 22.5 or 45 min (indicated below the blots) with I: Y. enterocolitica ΔHOPEMT asd carrying III: pBad_Si2, IV: YopE₁₋₁₃₈-SopE or V: YopE₁₋₁₃₈-SopB at an MOI of 100 (C) cAMP levels (in fmol/well shown on y-axis) in HeLa cells left untreated (I) or infected for 2.5 h with V: Y. enterocolitica ΔHOPEMT asd+YopE₁₋₁₃₈-BepA, VI: Y. enterocolitica ΔHOPEMT asd+YopE₁₋₁₃₈-BepA_{E305-end}, VII: Y. enterocolitica ΔHOPEMT asd+YopE₁₋₁₃₈-BepG_{Bid} or VIII: Y. enterocolitica ΔHOPEMT asd+pBad_Si2 at an MOI of 100. Cholera toxin (CT) was added for 1 h as positive control to samples II (1 μg/ml), III (25 μg/ml) or IV (50 μg/ml). Data were combined from n=3 independent experiments, error bars indicated are standard error of the mean. Statistical analysis was performed using an unpaired two-tailed t-test (ns indicates a non significant change, ** indicates a p value <0.01, *** indicates a p value<0.001).

FIG. 7: Delivery of human tBid into eukaryotic cells induces massive apoptosis. (A) Cleaved Caspase 3 p17 (“a”) and actin (“b”) western blot analysis on HeLa cells left untreated (II) or infected for 60 min with I: Y. enterocolitica ΔHOPEMT asd carrying III: pBad_Si2, IV: YopE₁₋₁₃₈-Bid or V: YopE₁₋₁₃₈-t-Bid at an MOI of 100. In some cases, cells were treated with VI: 0.5 μM Staurosporine or VII: 1 μM Staurosporine (B) Digitonin lysed HeLa cells left untreated (II) or after infection for 1 h with I: Y. enterocolitica ΔHOPEMT asd carrying III: pBad_Si2, IV: YopE₁₋₁₃₈-Bid or V: YopE₁₋₁₃₈-t-Bid at an MOI of 100 were analyzed by Western blotting anti-Bid (“a”) allowing comparison of endogenous Bid levels (marked Z) to translocated YopE₁₋₁₃₈-Bid (marked X) or YopE₁₋₁₃₈-tBid (marked Y) levels. As a loading control western blotting anti-Actin was performed (shown in “b”). In some cases, cells were treated with VI: 0.5 μM Staurosporine or VII: 1 μM Staurosporine (C) HeLa cells were left untreated (I) or infected at an MOI of 100 for 1 h with II: Y. enterocolitica ΔHOPEMT asd+pBad_Si2, III: Y. enterocolitica ΔHOPEMT asd+YopE₁₋₁₃₈-Bid, IV: Y. enterocolitica ΔHOPEMT asd+YopE₁₋₁₃₈-tBid. In some cases, cells were treated with V: 0.5 μM Staurosporine or VI: 1 μM Staurosporine. After fixation cells were stained for the actin cytoskeleton (gray).

FIG. 8: T3SS dependent delivery of zebrafish BIM induces apoptosis in zebrafish embryos. (A) 2 dpf zebrafish embryos were infected with the EGFP expressing Y. enterocolitica ΔHOPEMT asd+pBad_Si1 control strain (I) or zBIM translocating strain (II: Y. enterocolitica ΔHOPEMT asd+YopE₁₋₁₃₈-zBIM) by injection of about 400 bacteria into the hindbrain region. After 5.5 h the embryos were fixed, stained for activated Caspase 3 (cleaved Caspase 3, p17; shown in “c”) and analyzed for presence of bacteria (EGFP signal, shown in “b”). Maximum intensity z projections are shown for fluorescent images. Bright-field z projection are shown in “a” (B) Automated image analysis on maximum intensity z projections of recorded z-stack images of (A). Briefly, bacteria were detected via the GFP channel. Around each area of a bacterial spot a circle with a radius of 10 pixels was created. Overlapping regions were separated equally among the connecting members. In those areas closely surrounding bacteria, the Caspase 3 p17 staining intensity was measured and is plotted on the y-axis (as [a.u.]). Statistical analysis was performed using a Mann-Whitney test (*** indicates a p value<0.001). Data were combined from n=14 for Y. enterocolitica ΔHOPEMT asd+pBad_Si1 control strain (I) or n=19 for II: Y. enterocolitica ΔHOPEMT asd+YopE₁₋₁₃₈-zBIM infected animals, error bars indicated are standard error of the mean.

FIG. 9: tBiD dependent phosphoproteome: HeLa cells were infected for 30 min with Y. enterocolitica ΔHOPEMT asd+YopE₁₋₁₃₈-t-Bid at an MOI of 100 and as a control with Y. enterocolitica ΔHOPEMT asd+pBad_Si2. (A) Graphical representation of the tBID phosphoproteome. Proteins containing phosphopeptides that were significantly regulated in a tBid dependent manner (gray) (q-value<0.01) as well as known apopotosis related proteins (dark gray) are represented in a STRING network of known and predicted protein-protein interactions (high-confidence, score 0.7). Only proteins with at least one connection in STRING are represented. (B) Confocal images of HeLa cells infected with either Y. enterocolitica ΔHOPEMT asd+pBad_Si2 (I) or Y. enterocolitica ΔHOPEMT asd+YopE₁₋₁₃₈-t-Bid (II) reveal the induction of an apoptotic phenotype upon tBid delivery. Cells were stained for the nuclei with Hoechst (“a”), for F-actin with phalloidin (“b”), for tubulin with an anti-tubulin antibody (“c”) and for mitochondria with mitotracker (“d)”. Scale bar represents 40 μm.

FIG. 10: Description of the type III secretion-based delivery toolbox. (A) Vector maps of the cloning plasmids pBad_Si1 and pBad_Si2 used to generate fusion constructs with YopE₁₋₁₃₈. The chaperone SycE and the YopE₁₋₁₃₈-fusion are under the native Y. enterocolitica promoter. The two plasmids only differ in presence of an arabinose inducible EGFP present on pBad_Si1 (B) Multiple cloning site directly following the yopE₁₋₁₃₈ fragment on pBad_Si1 and pBad_Si2 plasmids.

FIG. 11: Characterization of T3SS protein delivery into various cell lines. Anti-Myc immunofluorescence staining on Swiss 3T3 fibroblasts (“a”), Jurkat cells (“b”) and HUVEC cells (“c”) left untreated (II) or infected with Y. enterocolitica ΔHOPEMT asd+pBad_Si2 (I) at the MOI indicated above the images (MOI 25, 50, 100, 200 and 400 for HUVECs) for 1 h.

FIG. 12: T3SS dependency of delivery of bacterial effector proteins into eukaryotic cell. Digitonin lysed HeLa cells after infection at an MOI of 100 for time indicated above the blots (0, 5, 15, 10, 60 and 120 minutes) with Y. enterocolitica ΔHOPEMT asd ΔyopB+YopE₁₋₁₃₈-SopE-Myc (I) or Y. enterocolitica ΔHOPEMT asd+YopE₁₋₁₃₈-SopE-Myc (II) were analyzed by Western blotting anti-Myc. The size corresponding to YopE₁₋₁₃₈-SopE-Myc is marked with “a”, while the size of the endogenous c-Myc protein is marked with “b”.

FIGS. 13 and 14: T3SS dependent secretion of various other proteins into the culture supernatant. In-vitro secretion experiment of I: Y. enterocolitica ΔHOPEMT asd+YopE₁₋₁₃₈ fused to the protein as indicated. Protein content of total bacterial lysates (“A”) and precipitated culture supernatants (“B”) was analyzed by Western blotting using an anti-YopE antibody. Numbers written indicate molecular weight in kDa at the corresponding height.

FIGS. 15A to M: Y. enterocolitica and S. enterica strains used in this study. List of Y. enterocolitica and S. enterica strains used in this study providing information on background strains, plasmids and proteins for T3SS dependent delivery encoded on corresponding plasmids. Further, information on oligonucleotides used for construction of the corresponding plasmid, the backbone plasmid and antibiotic resistances is provided.

FIG. 16: Delivery of murine tBid, murine Bid BH3 and murine Bax BH3 into B16F10 cells induces massive apoptosis. B16F10 cells uninfected (I) or after infection (MOI of 50) for 2.5 h with Y. enterocolitica ΔHOPEMT asd and II: +pBadSi_2, III: +YopE₁₋₁₃₈-Y. enterocolitica codon optimized murine tBid, IV: +YopE₁₋₁₃₈-Y. enterocolitica codon optimized murine Bid BH3 or V: +YopE₁₋₁₃₈-Y. enterocolitica codon optimized murine Bax BH3. After fixation cells were stained for the actin cytoskeleton and nuclei (both in gray).

FIG. 17: Delivery of murine tBid, murine Bid BH3 and murine Bax BH3 into D2A1 cells induces massive apoptosis. D2A1 cells uninfected (I) or after infection (MOI of 50) for 2.5 h with Y. enterocolitica ΔHOPEMT asd and II: +pBadSi_2, III: +YopE₁₋₁₃₈-Y. enterocolitica codon optimized murine tBid, IV: +YopE₁₋₁₃₈-Y. enterocolitica codon optimized murine Bid BH3 or V: +YopE₁₋₁₃₈-Y. enterocolitica codon optimized murine Bax BH3. After fixation cells were stained for the actin cytoskeleton and nuclei (both in gray).

FIG. 18: Delivery of murine tBid, murine Bid BH3 and murine Bax BH3 into Hela cells induces massive apoptosis. HeLa cells uninfected (I) or after infection (MOI of 50) for 2.5 h with Y. enterocolitica ΔHOPEMT asd and II: +pBadSi_2, III: +YopE₁₋₁₃₈-Y. enterocolitica codon optimized murine tBid, IV: +YopE₁₋₁₃₈-Y. enterocolitica codon optimized murine Bid BH3 or V: +YopE₁₋₁₃₈-Y. enterocolitica codon optimized murine Bax BH3. After fixation cells were stained for the actin cytoskeleton and nuclei (both in gray).

FIG. 19: Delivery of murine tBid, murine Bid BH3 and murine Bax BH3 into 4T1 cells induces massive apoptosis. 4Tl cells uninfected (I) or after infection (MOI of 50) for 2.5 h with Y. enterocolitica ΔHOPEMT asd and II: +pBadSi_2, III: +YopE₁₋₁₃₈-Y. enterocolitica codon optimized murine tBid, IV: +YopE₁₋₁₃₈-Y. enterocolitica codon optimized murine Bid BH3 or V: +YopE₁₋₁₃₈-Y. enterocolitica codon optimized murine Bax BH3. After fixation cells were stained for the actin cytoskeleton and nuclei (both in gray).

FIG. 20: Delivery of murine tBid by S. enterica grown under SPI-1 T3SS inducing conditions into eukaryotic cells induces apoptosis. Cleaved Caspase 3 p17 western blot analysis on HeLa cells left untreated (I) or infected for 4 h with III: S. enterica aroA carrying IV: SteA₁₋₂₀-t-Bid, V: SteA_{FL}-Bid, VI: SopE₁₋₈₁-t-Bid or VII: SopE₁₋₁₀₅-t-Bid at an MOI of 100. For this experiment, all S. enterica aroA strains were grown under SPI-1 T3SS inducing conditions. In some cases, cells were treated with II: 1 μM Staurosporine. Numbers written indicate molecular weight in kDa at the corresponding height.

FIG. 21: Delivery of murine tBid by S. enterica grown under SPI-2 T3SS inducing conditions into eukaryotic cells induces apoptosis. Cleaved Caspase 3 p17 western blot analysis on HeLa cells left untreated (I) or infected for 4 h with III: S. enterica aroA carrying IV: SteA₁₋₂₀-t-Bid, V: SteA_{FL}-Bid, VI: SopE₁₋₈₁-t-Bid or VII: SopE₁₋₁₀₅-t-Bid at an MOI of 100. For this experiment, all S. enterica aroA strains were grown under SPI-2 T3SS inducing conditions. In some cases, cells were treated with II: 1 μM Staurosporine. Numbers written indicate molecular weight in kDa at the corresponding height.

FIG. 22: S. enterica T3SS dependent secretion of various cell cycle proteins into the culture supernatant. In-vitro secretion experiment of S. enterica aroA+either SteA_{FL} (I, III, V, VII) or SopE₁₋₁₀₅ (II, IV, VI, VIII) fused to proteins as listed following. I and II: Ink4a-MycHis; III and IV: Ink4c-MycHis; V and VI: Mad2-MycHis; VII and VIII: Cdk1-MycHis. Protein content of precipitated culture supernatants (“A”) and total bacterial lysates (“B”) was analyzed by Western blotting using an anti-myc antibody. Numbers written indicate molecular weight in kDa at the corresponding height.

FIG. 23: T3SS dependent secretion of various known cell cycle interfering peptides into the culture supernatant. In-vitro secretion experiment of I: Y. enterocolitica ΔHOPEMT asd+pBad_Si2. II-VII: Y. enterocolitica ΔHOPEMT asd+YopE₁₋₁₃₈ fused to peptides as listed following: II: Ink4A₈₄₋₁₀₃; III: p107/RBL1₆₅₇₋₆₆₂; IV: p21_{141-160D149A}; V: p21_{145-160D149A}; VI: p21₁₇₋₃₃; VII: cyclin D2₁₃₉₋₁₄₇. Protein content of precipitated culture supernatants (“A”) and total bacterial lysates (“B”) was analyzed by Western blotting using an anti-YopE antibody. Numbers written indicate molecular weight in kDa at the corresponding height.

FIG. 24: Fusion of the T3SS delivered protein to Ubiquitin allows removal of the YopE₁₋₁₃₈ appendage. HeLa cells are infected with a strain delivering a protein of interest fused to YopE₁₋₁₃₈ with a directly fused Ubiquitin (YopE₁₋₁₃₈-Ubi). After protein delivery into the eukaryotic cell, endogenous Ubiquitin specific proteases will cleave the YopE₁₋₁₃₈-Ubi appendage from the protein of interest. Digitonin lysed HeLa cells uninfected (I) or after infection (MOI of 100) for 1 h with II: Y. enterocolitica ΔHOPEMT asd+YopE₁₋₁₃₈-Flag-INK4C-MycHis or III: +YopE₁₋₁₃₈-Flag-Ubiquitin-INK4C-MycHis were analyzed by Western blotting anti-INK4C for the presence of IV: YopE₁₋₁₃₈-Flag-Ubiquitin-INK4C-MycHis or V: YopE₁₋₁₃₈-Flag-INK4C-MycHis, the cleaved form VI: INK4C-MycHis and VII: the endogenous INK4C.

### DETAILED DESCRIPTION OF THE INVENTION

The present invention provides recombinant Gram-negative bacterial strains and the use thereof for delivery of heterologous proteins into eukaryotic cells.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

The term “Gram-negative bacterial strain” as used herein includes the following bacteria: Aeromonas salmonicida, Aeromonas hydrophila, Aeromonas veronii, Anaeromyxobacter dehalogenans, Bordetella bronchiseptica, Bordetella bronchiseptica, Bordetella parapertussis, Bordetella pertussis, Bradyrhizobium japonicum, Burkholderia cenocepacia, Burkholderia cepacia, Burkholderia mallei, Burkholderia pseudomallei, Chlamydia muridarum, Chlamydia trachmoatis, Chlamydophila abortus, Chlamydophila pneumoniae, Chromobacterium violaceum, Citrobacter rodentium, Desulfovibrio vulgaris, Edwardsiella tarda, Endozoicomonas elysicola, Erwinia amylovora, Escherichia albertii, Escherichia coli, Lawsonia intracellularis, Mesorhizobium loti, Myxococcus xanthus, Pantoea agglomerans, Photobacterium damselae, Photorhabdus luminescens, Photorabdus temperate, Pseudoalteromonas spongiae, Pseudomonas aeruginosa, Pseudomonas plecoglossicida, Pseudomonas syringae, Ralstonia solanacearum, Rhizobium sp, Salmonella enterica and other Salmonella sp, Shigella flexneri and other Shigella sp, Sodalis glossinidius, Vibrio alginolyticus, Vibrio azureus, Vibrio campellii, Vibrio caribbenthicus, Vibrio harvey, Vibrio parahaemolyticus, Vibrio tasmaniensis, Vibrio tubiashii, Xanthomonas axonopodis, Xanthomonas campestris, Xanthomonas oryzae, Yersinia enterocolitica, Yersinia pestis, Yersinia pseudotuberculosis. Preferred Gram-negative bacterial strains of the invention are Gram-negative bacterial strains comprised by the family of Enterobacteriaceae and Pseudomonadaceae. The Gram-negative bacterial strain of the present invention is normally used for delivery of heterologous proteins by the bacterial T3SS into eukaryotic cells in vitro and in vivo.

The term “recombinant Gram-negative bacterial strain” used herein refers to a Gram-negative bacterial strain genetically transformed with a vector. A useful vector of the present invention is e.g an expression vector, a vector for chromosomal or virulence plasmid insertion or a DNA fragment for chromosomal or virulence plasmid insertion.

The terms “Gram-negative bacterial strain deficient to produce an amino acid essential for growth” and “auxotroph mutant” are used herein interchangeably and refer to Gram-negative bacterial strains which can not grow in the absence of at least one exogenously provided essential amino acid or a precursor thereof. The amino acid the strain is deficient to produce is e.g. aspartate, meso-2,6-diaminopimelic acid, aromatic amino acids or leucine-arginine [23]. Such a strain can be generated by e.g. deletion of the aspartate-beta-semialdehyde dehydrogenase gene (Δasd). Such an auxotroph mutant cannot grow in absence of exogenous meso-2,6-diaminopimelic acid [24]. The mutation, e.g. deletion of the aspartate-beta-semialdehyde dehydrogenase gene is preferred herein for a Gram-negative bacterial strain deficient to produce an amino acid essential for growth of the present invention.

The term “Gram-negative bacterial strain deficient to produce adhesion proteins binding to the eukaryotic cell surface or extracellular matrix” refers to mutant Gram-negative bacterial strains which do not express at least one adhesion protein compared to the adhesion proteins expressed by the corresponding wild type strain. Adhesion proteins may include e.g. extended polymeric adhesion molecules like pili/fimbriae or non-fimbrial adhesins. Fimbrial adhesins include type-1 pili (such as E. coli Fim-pili with the FimH adhesin), P-pili (such as Pap-pili with the PapG adhesin from E. coli), type 4 pili (as pilin protein from e.g. P. aeruginosa) or curli (Csg proteins with the CsgA adhesin from S. enterica). Non-fimbrial adhesions include trimeric autotransporter adhesins such as YadA from Y. enterocolitica, BpaA (B. pseudomallei), Hia (H. influenzae), BadA (B. henselae), NadA (N. meningitidis) or UspA1 (M. catarrhalis) as well as other autotransporter adhesins such as AIDA-1 (E. coli) as well as other adhesins/invasins such as InvA from Y. enterocolitica or Intimin (E. coli) or members of the Dr-family or Afa-family (E. coli). The terms YadA and InvA as used herein refer to proteins from Y. enterocolitica. The autotransporter YadA [25, 26] binds to different froms of collagen as well as fibronectin, while the invasin InvA [27-29] binds to β-integrins in the eukaryotic cell membrane. If the Gram-negative bacterial strain is a Y. enterocolitica strain the strain is preferably deficient in InvA and/or YadA.

As used herein, the term “family of Enterobacteriaceae” comprises a family of gram-negative, rod-shaped, facultatively anaerobic bacteria found in soil, water, plants, and animals, which frequently occur as pathogens in vertebrates. The bacteria of this family share a similar physiology and demonstrate a conservation within functional elements and genes of the respective genomes. As well as being oxidase negative, all members of this family are glucose fermenters and most are nitrate reducers.

Enterobacteriaceae bacteria of the invention may be any bacteria from that family, and specifically includes, but is not limited to, bacteria of the following genera: Escherichia, Shigella, Edwardsiella, Salmonella, Citrobacter, Klebsiella, Enterobacter, Serratia, Proteus, Erwinia, Morganella, Providencia, or Yersinia. In more specific embodiments, the bacterium is of the Escherichia coli, Escherichia blattae, Escherichia fergusonii, Escherichia hermanii, Escherichia vuneris, Salmonella enterica, Salmonella bongori, Shigella dysenteriae, Shigella flexneri, Shigella boydii, Shigella sonnei, Enterobacter aerogenes, Enterobacter gergoviae, Enterobacter sakazakii, Enterobacter cloacae, Enterobacter agglomerans, Klebsiella pneumoniae, Klebsiella oxytoca, Serratia marcescens, Yersinia pseudotuberculosis, Yersinia pestis, Yersinia enterocolitica, Erwinia amylovora, Proteus mirabilis, Proteus vulgaris, Proteus penneri, Proteus hauseri, Providencia alcalifaciens, or Morganella morganii species. Preferably the Gram-negative bacterial strain is selected from the group consisting of the genera Yersinia, Escherichia, Salmonella, Shigella, Pseudomonas, Chlamydia, Erwinia, Pantoea, Vibrio, Burkholderia, Ralstonia, Xanthomonas, Chromobacterium, Sodalis, Citrobacter, Edwardsiella, Rhizobiae, Aeromonas, Photorhabdus, Bordetella and Desulfovibrio, more preferably from the group consisting of the genera Yersinia, Escherichia, Salmonella, and Pseudomonas, most preferably from the group consisting of the genera Yersinia and Salmonella.

The term “Yersinia” as used herein includes all species of Yersinia, including Yersinia enterocolitica, Yersinia pseudotuberculosis and Yersinia pestis. Prefered is Yersinia enterocolitica.

The term “Salmonella” as used herein includes all species of Salmonella, including Salmonella enterica and S. bongori. Prefered is Salmonella enterica.

“Promoter” as used herein refers to a nucleic acid sequence that regulates expression of a transcriptional unit. A “promoter region” is a regulatory region capable of binding RNA polymerase in a cell and initiating transcription of a downstream (3′ direction) coding sequence. Within the promoter region will be found a transcription initiation site (conveniently defined by mapping with nuclease S1), as well as protein binding domains (consensus sequences) responsible for the binding of RNA polymerase such as the putative-35 region and the Pribnow box. The term “operably linked” when describing the relationship between two DNA regions simply means that they are functionally related to each other and they are located on the same nucleic acid fragment. A promoter is operably linked to a structural gene if it controls the transcription of the gene and it is located on the same nucleic acid fragment as the gene. Usually the promoter is functional in said Gram-negative bacterial strain, i.e. the promoter is capable of expressing the fusion protein of the present invention, i.e. the promoter is capable of expressing the fusion protein of the present invention without further genetic engineering or expression of further proteins. Furthermore, a functional promoter must not be naturally counter-regulated to the bacterial T3SS.

The term “delivery” used herein refers to the transportation of a protein from a recombinant Gram-negative bacterial strain to a eukaryotic cell, including the steps of expressing the heterologous protein in the recombinant Gram-negative bacterial strain, secreting the expressed protein(s) from such Gram-negative bacterial strain and translocating the secreted protein(s) by such Gram-negative bacterial strain into the cytosol of the eukaryotic cell. Accordingly, the terms “delivery signal” or “secretion signal” which are used interchangeably herein refer to a polypeptide sequence which can be recognized by the secretion and translocation system of the Gram-negative bacterial strain and directs the delivery of a protein from the Gram-negative bacterial strain to eukaryotic cells.

As used herein, the “secretion” of a protein refers to the transportation of a heterologous protein outward across the cell membrane of a recombinant Gram-negative bacterial strain. The “translocation” of a protein refers to the transportation of a heterologous protein from a recombinant Gram-negative bacterial strain across the plasma membrane of a eukaryotic cell into the cytosol of such eukaryotic cell.

The term “eukaryotic cells” as used herein includes e.g. the following eukaryotic cells: Hi-5,HeLa, Hek, HUVECs, 3T3, CHO, Jurkat, Sf-9, HepG2, Vero, MDCK, Mefs, THP-1, J774, RAW, Caco2, NCI60, DU145, Lncap, MCF-7, MDA-MB-438, PC3, T47D, A549, U87, SHSY5Y, Ea.Hy926, Saos-2, 4T1, D2A1, B16F10, and primary human hepatocytes. “Eukaryotic cells” as used herein, are also referred to as “target cells” or “target eukaryotic cells”.

The term “T3SS effector protein” as used herein refers to proteins which are naturally injected by T3S systems into the cytosol of eukaryotic cells and to proteins which are naturally secreted by T3S systems that might e.g form the translocation pore into the eukaryotic membrane (including pore-forming tranlocators (as Yersinia YopB and YopD) and tip-proteins like Yersinia LcrV). Preferably proteins which are naturally injected by T3S systems into the cytosol of eukaryotic cells are used. These virulence factors will paralyze or reprogram the eukaryotic cell to the benefit of the pathogen. T3S effectors display a large repertoire of biochemical activities and modulate the function of crucial host regulatory molecules [5, 30] and include AvrA, AvrB, AvrBs2, AvrBS3, AvrBsT, AvrD, AvrD1, AvrPphB, AvrPphC, AvrPphEPto, AvrPpiBPto, AvrPto, AvrPtoB, AvrRpm1, AvrRpt2, AvrXv3, CigR, EspF, EspG, EspH, EspZ, ExoS, ExoT, GogB, GtgA, GtgE, GALA family of proteins, HopAB2, HopAO1, HopI1, HopM1, HopN1, HopPtoD2, HopPtoE, HopPtoF, HopPtoN, HopU1, HsvB, IcsB, IpaA, IpaB, IpaC, IpaH, IpaH7.8, IpaH9.8, IpgB1, IpgB2, IpgD, LcrV, Map, OspC1, OspE2, OspF, OspG, OspI, PipB, PipB2, PopB, PopP2, PthXo1 PthXo6, PthXo7, SifA, SifB, SipA/SspA, SipB, SipC/SspC, SipD/SspD, SlrP, SopA, SopB/SigD, SopD, SopE, SopE2, SpiC/SsaB, SptP, SpvB, SpvC, SrfH, SrfJ, Sse, SseB, SseC, SseD, SseF, SseG, SseI/SrfH, SseJ, SseK1, SseK2, SseK3, SseL, SspH1, SspH2, SteA, SteB, SteC, SteD, SteE, TccP2, Tir, VirA, VirPphA, VopF, XopD, YopB, YopD YopE, YopH, YopJ, YopM, YopO, YopP, YopT, YpkA.

T3SS effector genes of Yersinia have been cloned from e.g. Y. enterocolitica which are YopE, YopH, YopM, YopO, YopP/YopJ, and YopT [31]. The respective effector genes can be cloned from Shigella flexneri (e.g. OspF, IpgD, IpgB1), Salmonella enterica (e.g. SopE, SopB, SptP), P. aeruginosa (e.g ExoS, ExoT, ExoU, ExoY) or E. coli (e.g. Tir, Map, EspF, EspG, EspH, EspZ). The nucleic acid sequences of these genes are available to those skilled in the art, e.g., in the Genebank Database (yopH, yopO, yopE, yopP, yopM, yopT from NC_002120 GI:10955536; S. flexneri effector proteins from AF386526.1 GI:18462515; S. enterica effectors from NC_016810.1 GI:378697983 or FQ312003.1 GI:301156631; P. aeruginosa effectors from AE004091.2 GI:110227054 or CP000438.1 GI:115583796 and E. coli effector proteins from NC_011601.1 GI:215485161).

For the purpose of the present invention, genes are denoted by letters of lower case and italicised to be distinguished from proteins. In case the genes (denoted by letters of lower case and italicised) are following a bacterial species name (like E. coli), they refer to a mutation of the corresponding gene in the corresponding bacterial species. For example, YopE refers to the effector protein encoded by the yopE gene. Y. enterocolitica yopE represents a Y. enterocolitica having a mutaion in the yopE gene.

As used herein, the terms “polypeptide”, “peptide”, “protein”, “polypeptidic” and “peptidic” are used interchangeably to designate a series of amino acid residues connected to each other by peptide bonds between the alpha-amino and carboxy groups of adjacent residues. Preferred are proteins which have an amino acid sequence comprising at least 10 amino acids, more preferably at least 20 amino acids.

According to the present invention, “a heterologous protein” includes naturally occurring proteins or parts thereof and also includes artificially engineered proteins or parts thereof. As used herein, the term “heterologous protein” refers to a protein or a part thereof other than the T3SS effector protein or N-terminal fragment thereof to which it can be fused. In particular the heterologous protein as used herein refers to a protein or a part thereof, which do not belong to the proteome, i.e. the entire natural protein complement of the specific recombinant Gram-negative bacterial strain provided and used by the invention, e.g. which do not belong to the proteome, i.e. the entire natural protein complement of a specific bacterial strain of the genera Yersinia, Escherichia, Salmonella or Pseudomonas. Usually the heterologous protein is of animal origin including human origin. Preferably the heterologous protein is a human protein. More preferably the heterologous protein is selected from the group consisting of proteins involved in apoptosis or apoptosis regulation, cell cycle regulators, ankyrin repeat proteins, cell signaling proteins, reporter proteins, transcription factors, proteases, small GTPases, GPCR related proteins, nanobody fusion constructs and nanobodies, bacterial T3SS effectors, bacterial T4SS effectors and viral proteins. Particular preferably the heterologous protein is selected from the group consisting of proteins involved in apoptosis or apoptosis regulation, cell cycle regulators, ankyrin repeat proteins, reporter proteins, small GTPases, GPCR related proteins, nanobody fusion constructs, bacterial T3SS effectors, bacterial T4SS effectors and viral proteins. Even more particular preferred are heterologous proteins selected from the group consisting of proteins involved in apoptosis or apoptosis regulation, cell cycle regulators, and ankyrin repeat proteins. Most preferred are proteins involved in apoptosis or apoptosis regulation, like animal, preferably human heterologous proteins involved in apoptosis or apoptosis regulation

In some embodiments the vector of the Gram-neagtive bacterial strain of the present invention comprises two second DNA sequences encoding the identical or two different heterologous proteins fused independently from each other in frame to the 3′end of said first DNA sequence.

In some embodiments the vector of the Gram-neagtive bacterial strain of the present invention comprises three second DNA sequences encoding the identical or three different heterologous proteins fused independently from each other in frame to the 3′end of said first DNA sequence.

The heterologous protein expressed by the recombinant Gram-negative bacterial strain has usually a molecular weight of between 1 and 150 kD, preferably between 1 and 120 kD, more preferably between land 100 kDa, most preferably between 15 and 100 kDa.

According to the present invention “proteins involved in apoptosis or apoptosis regulation” include, but are not limited to, Bad, Bcl2, Bak, Bmt, Bax, Puma, Noxa, Bim, Bcl-XL, Apaf1, Caspase 9, Caspase 3, Caspase 6, Caspase 7, Caspase 10, DFFA, DFFB, ROCK1, APP, CAD, ICAD, CAD, EndoG, AIF, HtrA2, Smac/Diablo, Arts, ATM, ATR, Bok/Mtd, Bmf, Mcl-1(S), IAP family, LC8, PP2B, 14-3-3 proteins, PKA, PKC, PI3K, Erk1/2, p90RSK, TRAF2, TRADD, FADD, Daxx, Caspase8, Caspase2, RIP, RAIDD, MKK7, JNK, FLIPs, FKHR, GSK3, CDKs and their inhibitors like the INK4-family (p16(Ink4a), p15(Ink4b), p18(Ink4c), p19(Ink4d)), and the Cip1/Waf1/Kip1-2-family (p21(Cipl/Waf1), p27(Kip1), p57(Kip2). Preferably Bad, Bmt, Bcl2, Bak, Bax, Puma, Noxa, Bim, Bcl-XL, Caspase9, Caspase3, Caspase6, Caspase7, Smac/Diablo, Bok/Mtd, Bmf, Mcl-1(S), LC8, PP2B, TRADD, Daxx, Caspase8, Caspase2, RIP, RAIDD, FKHR, CDKs and their inhibitors like the INK4-family (p16(Ink4a), p15(Ink4b), p18(Ink4c), p19(Ink4d)), most preferably BIM, Bid, truncated Bid, FADD, Caspase 3 (and subunits thereof), Bax, Bad, Akt, CDKs and their inhibitors like the INK4-family (p16(Ink4a), p15(Ink4b), p18(Ink4c), p19(Ink4d)) are used [32-34]. Additionally proteins involved in apoptosis or apoptosis regulation include DIVA, Bcl-Xs, Nbk/Bik, Hrk/Dp5, Bid and tBid, Egl-1, Bcl-Gs, Cytochrome C, Beclin, CED-13, BNIP1, BNIP3, Bcl-B, Bcl-W, Ced-9, A1, NR13, Bfl-1, Caspase 1, Caspase 2, Caspase 4, Caspase 5, Caspase 8.

Proteins involved in apoptosis or apoptosis regulation are selected from the group consisting of pro-apoptotic proteins, anti-apoptotic proteins, inhibitors of apoptosis-prevention pathways and inhibitors of pro-survival signalling or pathways. Pro-apoptotic proteins comprise proteins selected form the group consisting of Bax, Bak, Diva, Bcl-Xs, Nbk/Bik, Hrk/Dp5, Bmf, Noxa, Puma, Bim, Bad, Bid and tBid, Bok, Apaf1, Smac/Diablo, BNIP1, BNIP3, Bcl-Gs, Beclin 1, Egl-1 and CED-13, Cytochrome C, FADD, the Caspase family, and CDKs and their inhibitors like the INK4-family (p16 (Ink4a), p15 (Ink4b), p18 (Ink4c), p19 (Ink4d)) or selected from the group consisting of Bax, Bak, Diva, Bcl-Xs, Nbk/Bik, Hrk/Dp5, Bmf, Noxa, Puma, Bim, Bad, Bid and tBid, Bok, Egl-1, Apaf1, Smac/Diablo, BNIP1, BNIP3, Bcl-Gs, Beclin 1, Egl-1 and CED-13, Cytochrome C, FADD, and the Caspase family. Preferred are Bax, Bak, Diva, Bcl-Xs, Nbk/Bik, Hrk/Dp5, Bmf, Noxa, Puma, Bim, Bad, Bid and tBid, Bok, Egl-1, Apaf1, BNIP1, BNIP3, Bcl-Gs, Beclin 1, Egl-1 and CED-13, Smac/Diablo, FADD, the Caspase family, CDKs and their inhibitors like the INK4-family (p16(Ink4a), p15(Ink4b), p18(Ink4c), p19(Ink4d)). Equally preferred are Bax, Bak, Diva, Bcl-Xs, Nbk/Bik, Hrk/Dp5,Bmf, Noxa, Puma, Bim, Bad, Bid and tBid, Bok, Apaf1, BNIP1, BNIP3, Bcl-Gs, Beclin 1, Egl-1 and CED-13, Smac/Diablo, FADD, the Caspase family.

Anti-apoptotic proteins comprise proteins selected form the group consisting of Bcl-2, Bcl-X1, Bcl-B, Bcl-W, Mcl-1, Ced-9, A1, NR13, IAP family and Bfl-1. Preferred are Bcl-2, Bcl-Xl, Bcl-B, Bcl-W, Mcl-1, Ced-9, A1, NR13 and Bfl-1.

Inhibitors of apoptosis-prevention pathways comprise proteins selected form the group consisting of Bad, Noxa and Cdc25A. Preferred are Bad and Noxa.

Inhibitors of pro-survival signalling or pathways comprise proteins selected form the group consisting of PTEN, ROCK, PP2A, PHLPP, JNK, p38. Preferred are PTEN, ROCK, PP2A and PHLPP.

In some embodiments the heterologous proteins involved in apoptosis or apoptosis regulation are selected from the group consisting of BH3-only proteins, caspases and intracellular signalling proteins of death receptor control of apoptosis. BH3-only proteins comprise proteins selected form the group consisting of Bad, BIM, Bid and tBid, Puma, Bik/Nbk, Bod, Hrk/Dp5, BNIP1, BNIP3, Bmf, Noxa, Mcl-1, Bcl-Gs, Beclin 1, Egl-1 and CED-13. Preferred are Bad, BIM, Bid and tBid.

Caspases comprise proteins selected form the group consisting of Caspase 1, Caspase 2, Caspase 3, Caspase 4, Caspase 5, Caspase 6, Caspase 7, Caspase 8, Caspase 9, Caspase 10.

Preferred are Caspase 3, Caspase 8 and Caspase9.

Intracellular signalling proteins of death receptor control of apoptosis comprise proteins selected form the group consisting of FADD, TRADD, ASC, BAP31, GULPI/CED-6, CIDEA, MFG-E8, CIDEC, RIPK1/RIP1, CRADD, RIPK3/RIP3, Crk, SHB, CrkL, DAXX, the 14-3-3 family, FLIP, DFF40 and 45, PEA-15, SODD. Preferred are FADD and TRADD.

In some embodiments two heterologous proteins involved in apoptosis or apoptosis regulation are comprised by the vector of the Gram-negative bacterial strain of the present invention, wherein one protein is a pro-apoptotic protein and the other protein is an inhibitor of apoptosis-prevention pathways or wherein one protein is a pro-apoptotic protein and the other protein is an inhibitor of pro-survival signalling or pathways.

Pro-apoptotic proteins encompassed by the present invention have usually an alpha helical structure, preferably a hydrophobic helix surrounded by amphipathic helices and ususally comprise at least one of BH1, BH2, BH3 or BH4 domaines, preferably comprise at least one BH3 domain. Usually pro-apoptotic proteins encompassed by the present invention have no enzymatic activity.

The term “protease cleavage site” as used herein refers to a specific amino acid motif within an amino acid sequence e.g. within an amino acid sequence of a protein or a fusion protein, which is cleaved by a specific protease, which recognizes the amino acid motif. For review see [35]. Examples of protease cleavage sites are amino acid motifs, which are cleaved by a protease selected from the group consisting of enterokinase (light chain), enteropeptidase, prescission protease, human rhinovirus protease (HRV 3C), TEV protease, TVMV protease, FactorXa protease and thrombin.

The following amino acid motif is recognized by the respective protease:
Asp-Asp-Asp-Asp-Lys: Enterokinase (light chain)/Enteropeptidase
Leu-Glu-Val-Leu-Phe-Gln/Gly-Pro: PreScission Protease/human Rhinovirus protease (HRV 3C)
Glu-Asn-Leu-Tyr-Phe-Gln-Ser and modified motifs based on the Glu-X-X-Tyr-X-Gln-Gly/Ser (where X is any amino acid) recognized by TEV protease (tobacco etch virus)
Glu-Thr-Val-Arg-Phe-Gln-Ser: TVMV protease
Ile-(Glu or Asp)-Gly-Arg: FactorXa protease
Leu-Val-Pro-Arg/Gly-Ser: Thrombin.

Encompassed by the protease cleavage sites as used herein is ubiquitin. Thus in some preferred embodiments ubiquitin is used as protease cleavage site, i.e. the third DNA sequence encodes ubiquitin as protease cleavage site, which can be cleaved by a specific ubiquitin processing proteases at the N-terminal site, e.g. which can be cleaved by a specific ubiquitin processing proteases called Deubiquitinating enzymes at the N-terminal site endogeneously in the cell where the fusion protein has been delivered to. Ubiquitin is processed at its C-terminus by a group of endogenous Ubiquitin-specific C-terminal proteases (Deubiquitinating enzymes, DUBs). The cleavage of Ubiquitin by DUBs is supposed to happen at the very C-terminus of Ubiquitin (after G76).

An “individual,” “subject” or “patient” is a vertebrate. In certain embodiments, the vertebrate is a mammal. Mammals include, but are not limited to, primates (including human and non-human primates) and rodents (e.g., mice and rats). In certain embodiments, a mammal is a human.

The term “mutation” is used herein as a general term and includes changes of both single base pair and multiple base pairs. Such mutations may include substitutions, frame-shift mutations, deletions, insertions and truncations.

The term “labelling molecule or an acceptor site for a labelling molecule” as used herein refers to a small chemical compound binding to a specific amino acid sequence resulting in fluorescence of the bound chemical compound, preferably coumarine ligase/coumarine acceptor site (and derivates thereof), resorufin ligase/resorufin acceptor site (and derivates thereof) and the tetra-Cysteine motif (as Cys-Cys-Pro-Gly-Cys-Cys and derivates thereof) in use with FlAsH/ReAsH dye (life technologies) or a fluorescent protein as Enhanced Green Fluorescent Protein (EGFP).

The term “nuclear localization signal” as used herein refers to an amino acid sequence that marks a protein for import into the nucleus of a eukaryotic cell and includes preferably a viral nuclear localization signal such as the SV40 large T-antigen derived NLS (PPKKKRKV).

The term “multiple cloning site” as used herein refers to a short DNA sequence containing several restriction sites for cleavage by restriction endonucleases such as AclI, HindIII, SspI, MluCI, Tsp509I, PciI, AgeI, BspMI, BfuAI, SexAI, MluI, BceAI, HpyCH4IV, HpyCH4III, BaeI, BsaXI, AflIII, SpeI, BsrI, BmrI, BglII, AfeI, AluI, StuI, ScaI, ClaI, BspDI, PI-SceI, NsiI, AseI, SwaI, CspCI, MfeI, BssSI, BmgBI, PmlI, DraIII, AleI, EcoP15I, PvuII, AlwNI, BtsIMutI, TspRI, NdeI, NlaIII, CviAII, FatI, MslI, FspEI, XcmI, BstXI, PfIMI, BccI, NcoI, BseYI, FauI, SmaI, XmaI, TspMI, Nt.CviPII, LpnPI, AciI, SacII, BsrBI, MspI, HpaII, ScrFI, BssKI, StyD4I, BsaJI, BslI, BtgI, NciI, AvrII, MnlI, BbvCI, Nb.BbvCI, Nt.BbvCI, SbfI, Bpu10I, Bsu36I, EcoNI, HpyAV, BstNI, PspGI, StyI, BcgI, PvuI, BstUI, EagI, RsrII, BsiEI, BsiWI, BsmBI, Hpy991, MspA1I, MspJI, SgrAI, BfaI, BspCNI, XhoI, EarI, AcuI, PstI, BpmI, DdeI, SfcI, AflII, BpuEI, SmlI, AvaI, BsoBI, MboII, BbsI, XmnI, BsmI, Nb.BsmI, EcoRI, HgaI, AatII, ZraI, Tth111I PflFI, PshAI, AhdI, DrdI, Eco53kI, SacI, BseRI, PleI, Nt.BstNBI, MlyI, HinfI, EcoRV, MboI, Sau3AI, DpnII BfuCI, DpnI, BsaBI, TfiI, BsrDI, Nb.BsrDI, BbvI, BtsI, Nb.BtsI, BstAPI, SfaNI, SphI, NmeAIII, NaeI, NgoMIV, BglI, AsiSI, BtgZI, HinP1I, HhaI, BssHII, NotI, Fnu4HI, Cac8I, MwoI, NheI, BmtI, SapI, BspQI, Nt.BspQI, BlpI, TseI, ApeKI, Bsp1286I, AlwI, Nt.AlwI, BamHI, FokI, BtsCI, HaeIII, PhoI, FseI, SfiI, NarI, KasI, SfoI, PluTI, AscI, EciI, BsmFI, ApaI, PspOMI, Sau96I, NlaIV, KpnI, Acc65I, BsaI, HphI, BstEII, AvaII, BanI, BaeGI, BsaHI, BanII, RsaI, CviQI, BstZ17I, BciVI, SalI, Nt.BsmAI, BsmAI, BcoDI, ApaLI, BsgI, AccI, Hpy166II, Tsp45I, HpaI, PmeI, HincII, BsiHKAI, ApoI, NspI, BsrFI, BstYI, HaeII, CviKI-1, EcoO109I, PpuMI, I-CeuI, SnaBI, I-SceI, BspHI, BspEI, MmeI, TaqαI, NruI, Hpy188I, Hpy188III, XbaI, BclI, HpyCH4V, FspI, PI-PspI, MscI, BsrGI, MseI, PacI, PsiI, BstBI, DraI, PspXI, BsaWI, BsaAI, EaeI, preferably XhoI, XbaI, HindIII, NcoI, NotI, EcoRI, EcoRV, BamHI, NheI, SacI, SalI, BstBI. The term “multiple cloning site” as used herein further refers to a short DNA sequence used for recombination events as e.g in Gateway cloning strategy or for methods such as Gibbson assembly or topo cloning.

The term “Yersinia wild type strain” as used herein refers to a naturally occuring variant (as Y. enterocolitica E40) or a naturally occuring variant containing genetic modifications allowing the use of vectors, such as deletion mutations in restriction endonucleases or antibiotic resistance genes (as Y. enterocolitica MRS40, the Ampicillin sensitive derivate of Y. enterocolitica E40) These strains contain chromosomal DNA as well as an unmodified virulence plasmid (called pYV).

The term “comprise” is generally used in the sense of include, that is to say permitting the presence of one or more features or components.

In one embodiment the present invention provides a recombinant Gram-negative bacterial strain, wherein the Gram-negative bacterial strain is selected from the group consisting of the genera Yersinia, Escherichia, Salmonella and Pseudomonas. In one embodiment the present invention provides a recombinant Gram-negative bacterial strain, wherein the Gram-negative bacterial strain is selected from the group consisting of the genera Yersinia and Salmonella. Preferably the Gram-negative bacterial strain is a Yersinia strain, more preferably a Yersinia enterocolitica strain. Most preferred is Yersinia enterocolitica E40 or Ampicilline sensitive derivates therof as Y. enterocolitica MRS40 as described in [36]. Also preferably the Gram-negative bacterial strain is a Salmonella strain, more preferably a Salmonella enterica strain. Most preferred is Salmonella enterica Serovar Typhimurium SL1344 as described by the Public health England culture collection (NCTC 13347).

In one embodiment of the present invention the delivery signal from a bacterial T3SS effector protein comprises a bacterial T3SS effector protein or a N-terminal fragment thereof wherein the T3SS effector protein or a N-terminal fragment thereof may comprise a chaperone binding site. A T3SS effector protein or a N-terminal fragment thereof which comprises a chaperone binding site is particular useful as delivery signal in the present invention. Preferred T3SS effector proteins or N-terminal fragments thereof are selected from the group consisting of SopE, SopE2, SptP, YopE, ExoS, SipA, SipB, SipD, SopA, SopB, SopD, IpgB1, IpgD, SipC, SifA, SseJ, Sse, SrfH, YopJ, AvrA, AvrBsT, YopT, YopH, YpkA, Tir, EspF, TccP2, IpgB2, OspF, Map, OspG, OspI, IpaH, SspHI, VopF, ExoS, ExoT, HopAB2, XopD, AvrRpt2, HopAO1, HopPtoD2, HopU1, GALA family of proteins, AvrBs2, AvrD1, AvrBS3, YopO, YopP, YopE, YopM, YopT, EspG, EspH, EspZ, IpaA, IpaB, IpaC, VirA, IcsB, OspC1, OspE2, IpaH9.8, IpaH7.8, AvrB, AvrD, AvrPphB, AvrPphC, AvrPphEPto, AvrPpiBPto, AvrPto, AvrPtoB, VirPphA, AvrRpm1, HopPtoE, HopPtoF, HopPtoN, PopB, PopP2, AvrBs3, XopD, and AvrXv3. More preferred T3SS effector proteins or N-terminal fragments thereof are selected from the group consisting of SopE, SptP, YopE, ExoS, SopB, IpgB1, IpgD, YopJ, YopH, EspF, OspF, ExoS, YopO, YopP, YopE, YopM, YopT, whereof most preferred T3SS effector proteins or N-terminal fragments thereof are selected from the group consisting of IpgB1, SopE, SopB, SptP, OspF, IpgD, YopH, YopO, YopP, YopE, YopM, YopT, in particular YopE or an N-terminal fragment thereof.

Equally preferred T3SS effector proteins or N-terminal fragments thereof are selected from the group consisting of SopE, SopE2, SptP, SteA, SipA, SipB, SipD, SopA, SopB, SopD, IpgB1, IpgD, SipC, SifA, SifB, SseJ, Sse, SrfH, YopJ, AvrA, AvrBsT, YopH, YpkA, Tir, EspF, TccP2, IpgB2, OspF, Map, OspG, OspI, IpaH, VopF, ExoS, ExoT, HopAB2, AvrRpt2, HopAO1, HopU1, GALA family of proteins, AvrBs2, AvrD1, YopO, YopP, YopE, YopT, EspG, EspH, EspZ, IpaA, IpaB, IpaC, VirA, IcsB, OspC1, OspE2, IpaH9.8, IpaH7.8, AvrB, AvrD, AvrPphB, AvrPphC, AvrPphEPto, AvrPpiBPto, AvrPto, AvrPtoB, VirPphA, AvrRpm1, HopPtoD2, HopPtoE, HopPtoF, HopPtoN, PopB, PopP2, AvrBs3, XopD, and AvrXv3. Equally more preferred T3SS effector proteins or N-terminal fragments thereof are selected from the group consisting of SopE, SptP, SteA, SifB, SopB, IpgB1, IpgD, YopJ, YopH, EspF, OspF, ExoS, YopO, YopP, YopE, YopT, whereof equally most preferred T3SS effector proteins or N-terminal fragments thereof are selected from the group consisting of IpgB1, SopE, SopB, SptP, SteA, SifB, OspF, IpgD, YopH, YopO, YopP, YopE, and YopT, in particular SopE, SteA, or YopE or an N-terminal fragment thereof, more particular SteA or YopE or an N-terminal fragment thereof, most particular YopE or an N-terminal fragment thereof.

In some embodiments the delivery signal from the bacterial T3SS effector protein encoded by the first DNA sequence comprises the bacterial T3SS effector protein or an N-terminal fragment thereof, wherein the N-terminal fragment thereof includes at least the first 10, preferably at least the first 20, more preferably at least the first 100 amino acids of the bacterial T3SS effector protein.

In some embodiments the delivery signal from the bacterial T3SS effector protein encoded by the first DNA sequence comprises the bacterial T3SS effector protein or an N-terminal fragment thereof, wherein the bacterial T3SS effector protein or the N-terminal fragment thereof comprises a chaperone binding site.

Preferred T3SS effector proteins or a N-terminal fragment thereof, which comprise a chaperone binding site comprise the following combinations of chaperone binding site and T3SS effector protein or N-terminal fragment thereof: SycE-YopE, InvB-SopE, SicP-SptP, SycT-YopT, SycO-YopO, SycN/YscB-YopN, SycH-YopH, SpcS-ExoS, CesF-EspF, SycD-YopB, SycD-YopD. More preferred are SycE-YopE, InvB-SopE, SycT-YopT, SycO-YopO, SycN/YscB-YopN, SycH-YopH, SpcS-ExoS, CesF-EspF. Most preferred is a YopE or an N-terminal fragment thereof comprising the SycE chaperone binding site such as an N-terminal fragment of a YopE effector protein containing the N-terminal 138 amino acids of the YopE effector protein designated herein as YopE1-138 and as shown in SEQ ID NO. 2 or a SopE or an N-terminal fragment thereof comprising the InvB chaperone binding site s as uch an N-terminal fragment of a SopE effector protein containing the N-terminal 81 or 105 amino acids of the SopE effector protein designated herein as SopE₁₋₈₁ or SopE₁₋₁₀₅ respectively, and as shown in SEQ ID NO. 142 or 143.

In one embodiment of the present invention the recombinant Gram-negative bacterial strain is a Yersinia strain and the delivery signal from the bacterial T3SS effector protein encoded by the first DNA sequence comprises a YopE effector protein or an N-terminal part, preferably the Y. enterocolitica YopE effector protein or an N-terminal part thereof. Preferably the SycE binding site is comprised within the N-terminal part of the YopE effector protein. In this connection an N-terminal fragment of a YopE effector protein may comprise the N-terminal 12, 16, 18, 52, 53, 80 or 138 amino acids [10, 37, 38]. Most preferred is an N-terminal fragment of a YopE effector protein containing the N-terminal 138 amino acids of the YopE effector protein e.g. as described in Forsberg and Wolf-Watz [39] designated herein as YopE₁₋₁₃₈ and as shown in SEQ ID NO. 2.

In one embodiment of the present invention the recombinant Gram-negative bacterial strain is a Salmonella strain and the delivery signal from the bacterial T3SS effector protein encoded by the first DNA sequence comprises a SopE or SteA effector protein or an N-terminal part thereof, preferably the Salmonella enterica SopE or SteA effector protein or an N-terminal part thereof. Preferably the chaperon binding site is comprised within the N-terminal part of the SopE effector protein. In this connection an N-terminal fragment of a SopE effector protein protein may comprise the N-terminal 81 or 105 amino acids. Most preferred is the full length SteA and an N-terminal fragment of a SopE effector protein containing the N-terminal 105 amino acids of the effector protein e.g. as described in SEQ ID NO. 142 or 143.

One skilled in the art is familiar with methods for identifying the polypeptide sequences of an effector protein that are capable of delivering a protein. For example, one such method is described by Sory et al. [13]. Briefly, polypeptide sequences from e.g. various portions of the Yop proteins can be fused in-frame to a reporter enzyme such as the calmodulin-activated adenylate cyclase domain (or Cya) of the Bordetella pertussis cyclolysin. Delivery of a Yop-Cya hybrid protein into the cytosol of eukaryotic cells is indicated by the appearance of cyclase activity in the infected eukaryotic cells that leads to the accumulation of cAMP. By employing such an approach, one skilled in the art can determine, if desired, the minimal sequence requirement, i.e., a contiguous amino acid sequence of the shortest length, that is capable of delivering a protein, see, e.g. [13]. Accordingly, preferred delivery signals of the present invention consists of at least the minimal sequence of amino acids of a T3SS effector protein that is capable of delivering a protein.

In one embodiment the present invention provides mutant recombinant Gram-negative bacterial strains in particular recombinant Gram-negative bacterial strains which are deficient in producing at least one T3SS functional effector protein.

According to the present invention, such a mutant Gram-negative bacterial strain e.g. such a mutant Yersinia strain can be generated by introducing at least one mutation into at least one effector-encoding gene. Preferably, such effector-encoding genes include YopE, YopH, YopO/YpkA, YopM, YopP/YopJ and YopT as far as a Yersinia strain is concerned. Preferably, such effector-encoding genes include AvrA, CigR, GogB, GtgA, GtgE, PipB, SifB, SipA/SspA, SipB, SipC/SspC, SipD/SspD, SlrP, SopB/SigD, SopA, SpiC/SsaB, SseB, SseC, SseD, SseF, SseG, Ssel/SrfH, SopD, SopE, SopE2, SspH1, SspH2, PipB2, SifA, SopD2, SseJ, SseK1, SseK2, SseK3, SseL, SteC, SteA, SteB, SteD, SteE, SpvB, SpvC, SpvD, SrfJ, SptP, as far as a Salmonella strain is concerned. Most preferably, all effector-encoding genes are deleted. The skilled artisan may employ any number of standard techniques to generate mutations in these T3SS effector genes. Sambrook et al. describe in general such techniques. See Sambrook et al. [40].

In accordance with the present invention, the mutation can be generated in the promoter region of an effector-encoding gene so that the expression of such effector gene is abolished. The mutation can also be generated in the coding region of an effector-encoding gene such that the catalytic activity of the encoded effector protein is abolished. The “catalytic activity” of an effector protein refers normally to the anti-target cell function of an effector protein, i.e., toxicity. Such activity is governed by the catalytic motifs in the catalytic domain of an effector protein. The approaches for identifying the catalytic domain and/or the catalytic motifs of an effector protein are well known by those skilled in the art. See, for example, [41, 42].

Accordingly, one preferred mutation of the present invention is a deletion of the entire catalytic domain. Another preferred mutation is a frameshift mutation in an effector-encoding gene such that the catalytic domain is not present in the protein product expressed from such “frameshifted” gene. A most preferred mutation is a mutation with the deletion of the entire coding region of the effector protein. Other mutations are also contemplated by the present invention, such as small deletions or base pair substitutions, which are generated in the catalytic motifs of an effector protein leading to destruction of the catalytic activity of a given effector protein.

The mutations that are generated in the genes of the T3SS functional effector proteins may be introduced into the particular strain by a number of methods. One such method involves cloning a mutated gene into a “suicide” vector which is capable of introducing the mutated sequence into the strain via allelic exchange. An example of such a “suicide” vector is described by [43].

In this manner, mutations generated in multiple genes may be introduced successively into a Gram-negative bacterial strain giving rise to polymutant, e.g a sixtuple mutant recombinant strain. The order in which these mutated sequences are introduced is not important. Under some circumstances, it may be desired to mutate only some but not all of the effector genes. Accordingly, the present invention further contemplates polymutant Yersinia other than sixtuple-mutant Yersinia, e.g., double-mutant, triple-mutant, quadruple-mutant and quintuple-mutant strains. For the purpose of delivering proteins, the secretion and translocation system of the instant mutant strain needs to be intact.

A preferred recombinant Gram-negative bacterial strain of the present invention is a sixtuple-mutant Yersinia strain in which all the effector-encoding genes are mutated such that the resulting Yersinia no longer produce any functional effector proteins. Such sixtuple-mutant Yersinia strain is designated as ΔyopH,O,P,E,M,T for Y. enterocolitica. As an example such a sixtuple-mutant can be produced from the Y. enterocolitica MRS40 strain giving rise to Y. enterocolitica MRS40 ΔyopH,O,P,E,M,T, which is preferred.

A further aspect of the present invention is directed to a vector for use in combination with the recombinant Gram-negative bacterial strains to deliver a desired protein into eukaryotic cells, wherein the vector comprises in the 5′ to 3′ direction:
a promoter;
a first DNA sequence encoding a delivery signal from a bacterial T3SS effector protein, operably linked to said promoter;
a second DNA sequence encoding a heterologous protein fused in frame to the 3′end of said first DNA sequence; and alternatively
a third DNA sequence encoding a protease cleavage site, wherein the third DNA sequence is located between the 3′end of said first DNA sequence and the 5′end of said second DNA sequence.

Promoter, heterologous protein and protease cleavage site as described supra can be used for the vector of the Gram-negative bacterial strain.

Vectors which can be used according to the invention depend on the Gram-negative bacterial strains used as known to the skilled person. Vectors which can be used according to the invention include expression vectors, vectors for chromosomal or virulence plasmid insertion and DNA fragments for chromosomal or virulence plasmid insertion. Expression vectors which are useful in e.g. Yersinia, Escherichia, Salmonella or Pseudomonas strain are e.g pUC, pBad, pACYC, pUCP20 and pET plasmids. Vectors for chromosomal or virulence plasmid insertion which are useful in e.g. Yersinia, Escherichia, Salmonella or Pseudomonas strain are e.g pKNG101. DNA fragments for chromosomal or virulence plasmid insertion refer to methods used in e.g. Yersinia, Escherichia, Salmonella or Pseudomonas strain as e.g. lambda-red genetic engineering. Vectors for chromosomal or virulence plasmid insertion or DNA fragments for chromosomal or virulence plasmid insertion may insert the first, second and/or third DNA sequence of the present invention so that the first, second and/or third DNA sequence is operably linked to an endogenous promoter of the recombinant Gram-negative bacterial strain. Thus if a vector for chromosomal or virulence plasmid insertion or a DNA fragment for chromosomal or virulence plasmid insertion is used, an endogenous promoter can be encoded on the endogenous bacterial DNA (chromosomal or plasmid DNA) and only the first and second DNA sequence will be provided by the engineered vector for chromosomal or virulence plasmid insertion or DNA fragment for chromosomal or virulence plasmid insertion. Thus a promoter is not necessarily needed to be comprised by the vector used for transformation of the recombinant Gram-negative bacterial strains i.e. the recombinant Gram-negative bacterial strains of the present invention may be transformed with a vector which dose not comprise a promoter. Preferably an expression vector is used. The vector of the present invention is normally used for delivery of the heterologous proteins by the bacterial T3SS into eukaryotic cells in vitro and in vivo.

A preferred expression vector for Yersinia is selected from the group consisting of pBad_Si_1 and pBad_Si_2. pBad_Si2 was constructed by cloning of the SycE-YopE₁₋₁₃₈ fragment containing endogenous promoters for YopE and SycE from purified pYV40 into KpnI/HindIII site of pBad-MycHisA (Invitrogen). Additional modifications include removal of the NcoI/BglII fragment of pBad-MycHisA by digest, Klenow fragment treatment and religation. Further at the 3′ end of YopE₁₋₁₃₈ the following cleavage sites were added: XbaI-XhoI-BstBI-(HindIII). pBad_Si1 is equal to pBad_Si2 but encodes EGFP amplified from pEGFP-C1 (Clontech) in the NcoI/BglII site under the Arabinose inducible promoter.

A preferred expression vector for Salmonella is selected from the group consisting of pSi_266, pSi_267, pSi_268 and pSi_269. Plasmids pSi_266, pSi_267, pSi_268 and pSi_269 containing the corresponding endogenous promoter and the SteA₁₋₂₀ fragment (pSi_266), the full length SteA sequence (pSi_267), the SopE₁₋₈₁ fragment (pSi_268) or the SopE₁₋₁₀₅ fragment (pSi_269) were amplified from S. enterica SL1344 genomic DNA and cloned into NcoI/KpnI site of pBad-MycHisA (Invitrogen).

The vectors of the instant invention may include other sequence elements such as a 3′ termination sequence (including a stop codon and a poly A sequence), or a gene conferring a drug resistance which allows the selection of transformants having received the instant vector. The vectors of the present invention may be transformed by a number of known methods into the recombinant Gram-negative bacterial strains. For the purpose of the present invention, the methods of transformation for introducing a vector include, but are not limited to, electroporation, calcium phosphate mediated transformation, conjugation, or combinations thereof. For example, a vector can be transformed into a first bacteria strain by a standard electroporation procedure. Subsequently, such a vector can be transferred from the first bacteria strain into the desired strain by conjugation, a process also called “mobilization”.

Transformant (i.e., Gram-negative bacterial strains having taken up the vector) may be selected, e.g., with antibiotics. These techniques are well known in the art. See, for example, [13].

In accordance with the present invention, the promoter of the expression vector of the recombinant Gram-negative bacterial strain of the invention can be a native promoter of a T3SS effector protein of the respective strain or a compatible bacterial strain or a promoter used in expression vectors which are useful in e.g. Yersinia, Escherichia, Salmonella or Pseudomonas strain e.g pUC and pBad. Such promoters are the T7 promoter, Plac promoter or Ara-bad promoter.

If the recombinant Gram-negative bacterial strain is a Yersinia strain the promoter can be from a Yersinia virulon gene. A “Yersinia virulon gene” refers to genes on the Yersinia pYV plasmid, the expression of which is controlled both by temperature and by contact with a target cell. Such genes include genes coding for elements of the secretion machinery (the Ysc genes), genes coding for translocators (YopB, YopD, and LcrV), genes coding for the control elements (YopN, TyeA and LcrG), genes coding for T3SS effector chaperones (SycD, SycE, SycH, SycN, SycO and SycT), and genes coding for effectors (YopE, YopH, YopO/YpkA, YopM, YopT and YopP/YopJ) as well as other pYV encoded proteins as VirF and YadA. In a preferred embodiment of the present invention, the promoter is the native promoter of a T3SS functional effector encoding gene. If the recombinant Gram-negative bacterial strain is a Yersinia strain the promoter is selected from any one of YopE, YopH, YopO/YpkA, YopM and YopP/YopJ. More preferably, the promoter is from YopE or SycE.

If the recombinant Gram-negative bacterial strain is a Salmonella strain the promoter can be from SpiI or SpiII pathogenicity island or from an effector protein elsewhere encoded. Such genes include genes coding for elements of the secretion machinery, genes coding for translocators, genes coding for the control elements, genes coding for T3SS effector chaperones, and genes coding for effectors as well as other proteins encoded by SPI-1 or SPI-2. In a preferred embodiment of the present invention, the promoter is the native promoter of a T3SS functional effector encoding gene. If the recombinant Gram-negative bacterial strain is a Salmonella strain the promoter is selected from any one of the effector proteins. More preferably, the promoter is from SopE, InvB or SteA.

In a preferred embodiment the expression vector comprises a DNA sequence encoding a protease cleavage site. Generation of a functional and generally applicable cleavage site allows cleaving off the delivery signal after translocation. As the delivery signal can interfere with correct localization and/or function of the translocated protein within the target cells the introduction of a protease cleavage site between the delivery signal and the protein of interest provides for the first time delivery of almost native proteins into eukaryotic cells. Preferably the protease cleavage site is an amino acid motif which is cleaved by a protease or the catalytic domains thereof selected from the group consisting of enterokinase (light chain), enteropeptidase, prescission protease, human rhinovirus protease 3C, TEV protease, TVMV protease, FactorXa protease and thrombin, more preferably an amino acid motif which is cleaved by TEV protease. Equally preferable the protease cleavage site is an amino acid motif which is cleaved by a protease or the catalytic domains thereof selected from the group consisting of enterokinase (light chain), enteropeptidase, prescission protease, human rhinovirus protease 3C, TEV protease, TVMV protease, FactorXa protease, ubiquitin processing protease, called Deubiquitinating enzymes, and thrombin. Most preferred is an amino acid motif which is cleaved by TEV protease or by an ubiquitin processing protease. Thus in a further embodiment of the present invention, the heterologous protein is cleaved from the delivery signal from a bacterial T3SS effector protein by a protease. Preferred methods of cleavage are methods wherein:
a) the protease is translocated into the eukaryotic cell by a recombinant Gram-negative bacterial strain as described herein which expresses a fusion protein which comprises the delivery signal from the bacterial T3SS effector protein and the protease as heterologous protein; or
b) the protease is expressed constitutively or transiently in the eukaryotic cell. Usually the recombinant Gram-negative bacterial strain used to deliver a desired protein into a eukaryotic cell and the recombinant Gram-negative bacterial strain translocating the protease into the eukaryotic cell are different.

In one embodiment of the present invention the vector comprises a further DNA sequence encoding a labelling molecule or an acceptor site for a labelling molecule. The further DNA sequence encoding a labelling molecule or an acceptor site for a labelling molecule is usually fused to the 5′ end or to the 3′ end of the second DNA sequence. A preferred labelling molecule or an acceptor site for a labelling molecule is selected from the group consisting of enhanced green fluourescent protein (EGFP), coumarin, coumarin ligase acceptor site, resorufin, resurofin ligase acceptor site, the tetra-Cysteine motif in use with FlAsH/ReAsH dye (life technologies). Most preferred is resorufin and a resurofin ligase acceptor site or EGFP. The use of a labelling molecule or an acceptor site for a labelling molecule will lead to the attachment of a labelling molecule to the heterologous protein of interest, which will then be delivered as such into the eukaryotic cell and enables tracking of the protein by e.g. live cell microscopy.

In one embodiment of the present invention the vector comprises a further DNA sequence encoding a peptide tag. The further DNA sequence encoding a peptide tag is usually fused to the 5′ end or to the 3′ end of the second DNA sequence. A preferred peptide tag is selected from the group consisting of Myc-tag, His-tag, Flag-tag, HA tag, Strep tag or V5 tag or a combination of two or more tags out of these groups. Most preferred is Myc-tag, Flag-tag, His-tag and combined Myc-and His-tags. The use of a peptide tag will lead to traceability of the tagged protein e.g by immunofluorescence or Western blotting using anti-tag antibodies. Further, the use of a peptide tag allows affinity purification of the desired protein either after secretion into the culture supernatant or after translocation into eukaryotic cells, in both cases using a purification method suiting the corresponding tag (e.g. metal-chelate affinity purification in use with a His-tag or anti-Flag antibody based purification in use with the Flag-tag).

In one embodiment of the present invention the vector comprises a further DNA sequence encoding a nuclear localization signal (NLS). The further DNA sequence encoding a nuclear localization signal (NLS) is usually fused to the 5′end or to the 3′end of the second DNA sequence wherein said further DNA sequence encodes a nuclear localization signal (NLS). A preferred NLS is selected from the group consisting of SV40 large T-antigen NLS and

derivates thereof [44] as well as other viral NLS. Most preferred is SV40 large T-antigen NLS and derivates thereof.

In one embodiment of the present invention the vector comprises a multiple cloning site. The multiple cloning site is usually located at the 3′end of the first DNA sequence and/or at the 5′end or 3′end of the second DNA sequence. One or more than one multiple cloning sites can be comprised by the vector. A preferred multiple cloning site is selected from the group of restriction enzymes consisting of XhoI, XbaI, HindIII, NcoI, NotI, EcoRI, EcoRV, BamHI, NheI, SacI, SalI, BstBI. Most preferred is XbaI, XhoI, BstBI and HindIII.

The protein expressed from the fused first and second and optional third DNA sequences of the vector is also termed as a “fusion protein” or a “hybrid protein”, i.e., a fused protein or hybrid of delivery signal and a heterologous protein. The fusion protein can also comprise e.g. a delivery signal and two or more different heterologous proteins.

The present invention contemplates a method for delivering heterologous proteins as hereinabove described into eukaryotic cells in cell culture as well as in-vivo.

Thus in one embodiment the method for delivering heterologous proteins comprises
i) culturing the Gram-negative bacterial strain as described herein;
ii) contacting a eukaryotic cell with the Gram-negative bacterial strain of i) wherein a fusion protein which comprises a delivery signal from a bacterial T3SS effector protein and the heterologous protein is expressed by the Gram-negative bacterial strain and is translocated into the eukaryotic cell; and optionally
iii) cleaving the fusion protein so that the heterologous protein is cleaved from the delivery signal from the bacterial T3SS effector protein.

In some embodiments at least two fusion proteins which comprises each a delivery signal from a bacterial T3SS effector protein and a heterologous protein are expressed by the Gram-negative bacterial strain and are translocated into the eukaryotic cell by the methods of the present inventions.

The recombinant Gram-negative bacterial strain can be cultured so that a fusion protein is expressed which comprises the delivery signal from the bacterial T3SS effector protein and the heterologous protein according to methods known in the art (e.g. FDA, Bacteriological Analytical Manual (BAM), chapter 8: Yersinia enterocolitica). Preferably the recombinant Gram-negative bacterial strain can be cultured in Brain Heart infusion broth e.g. at 28° C. For induction of expression of T3SS and e.g. YopE/SycE promoter dependent genes, bacteria can be grown at 37° C.

In a preferred embodiment, the eukaryotic cell is contacted with two Gram-negative bacterial strains of i), wherein the first Gram-negative bacterial strain expresses a first fusion protein which comprises the delivery signal from the bacterial T3SS effector protein and a first heterologous protein and the second Gram-negative bacterial strain expresses a second fusion protein which comprises the delivery signal from the bacterial T3SS effector protein and a second heterologous protein, so that the first and the second fusion protein are translocated into the eukaryotic cell. This embodiment provided for co-infection of e.g eukaryotic cells with two bacterial strains as a valid method to deliver e.g. two different hybrid proteins into single cells to address their functional interaction.

The present invention contemplates a wide range of eukaryotic cells that may be targeted by the instant recombinant Gram-negative bacterial strain e.g. Hi-5 (BTI-TN-5B1-4; life technologies B855-02), HeLa cells, e.g. HeLa Cc12 (as ATCC No. CCL-2), fibroblast cells, e.g. 3T3 fibroblast cells (as ATCC No. CCL-92) or Mef (as ATCC No. SCRC-1040), Hek (as ATCC No. CRL-1573), HUVECs (as ATCC No. PCS-100-013), CHO (as ATCC No. CCL-61), Jurkat (as ATCC No. TIB-152), Sf-9 (as ATCC No. CRL-1711), HepG2 (as ATCC No. HB-8065), Vero (as ATCC No. CCL-81), MDCK (as ATCC No. CCL-34), THP-1 (as ATCC No. TIB-202), J774 (as ATCC No. TIB-67), RAW (as ATCC No. TIB-71), Caco2 (as ATCC No. HTB-37), NCI cell lines (as ATCC No. HTB-182), DU145 (as ATCC No. HTB-81), Lncap (as ATCC No. CRL-1740), MCF-7 (as ATCC No. HTB-22), MDA-MB cell lines (as ATCC No. HTB-128), PC3 (as ATCC No. CRL-1435), T47D (as ATCC No. CRL-2865), A549 (as ATCC No. CCL-185), U87 (as ATCC No. HTB-14), SHSY5Y (as ATCC No. CRL-2266s), Ea.Hy926 (as ATCC No. CRL-2922), Saos-2 (as ATCC No. HTBH-85), 4T1 (as ATCC No. CRL-2539), B16F10 (as ATCC No. CRL-6475), or primary human hepatocytes (as life technologies HMCPIS), preferably HeLa, Hek, HUVECs, 3T3, CHO, Jurkat, Sf-9, HepG2 Vero, THP-1, Caco2, Mef, A549, 4T1, B16F10 and primary human hepatocytes and most preferably HeLa, Hek, HUVECs, 3T3, CHO, Jurkat, THP-1, A549 and Mef. By “target”, is meant the extracellular adhesion of the recombinant Gram-negative bacterial strain to a eukaryotic cell.

In accordance with the present invention, the delivery of a protein can be achieved by contacting a eukaryotic cell with a recombinant Gram-negative bacterial strain under appropriate conditions. Various references and techniques are conventionally available for those skilled in the art regarding the conditions for inducing the expression and translocation of virulon genes, including the desired temperature, Ca⁺⁺ concentration, addition of inducers as Congo Red, manners in which the recombinant Gram-negative bacterial strain and target cells are mixed, and the like. See, for example, [45]. The conditions may vary depending on the type of eukaryotic cells to be targeted and the recombinant bacterial strain to be used. Such variations can be addressed by those skilled in the art using conventional techniques. Those skilled in the art can also use a number of assays to determine whether the delivery of a fusion protein is successful. For example, the fusion protein may be detected via immunofluorescence using antibodies recognizing a fused tag (like Myc-tag). The determination can also be based on the enzymatic activity of the protein being delivered, e.g., the assay described by [13].

In one embodiment the present invention provides a method of purifying a heterologous protein comprising culturing the Gram-negative bacterial strain as described herein so that a fusion protein which comprises a delivery signal from a bacterial T3SS effector protein and the heterologous protein is expressed and secreted into the supernatant of the culture. The fusion protein expressed may further comprise a protease cleavage site between the delivery signal from the bacterial T3SS effector protein and the heterologous protein and/or may further comprise a peptide tag.

Thus in a particular embodiment the method of purifying a heterologous protein comprises
i) culturing the Gram-negative bacterial strain as described herein so that a fusion protein which comprises a delivery signal from a bacterial T3SS effector protein, the heterologous protein and a protease cleavage site between the delivery signal from the bacterial T3SS effector protein and the heterologous protein is expressed and secreted into the supernatant of the culture;
ii) adding a protease to the supernatant of the culture wherein the protease cleaves the fusion protein so that the heterologous protein is cleaved from the delivery signal from the bacterial T3SS effector protein;
iii) optionally isolating the heterologous protein from the supernatant of the culture

Thus in another particular embodiment the method of purifying a heterologous protein comprises
i) culturing the Gram-negative bacterial strain as described herein so that a fusion protein which comprises a delivery signal from a bacterial T3SS effector protein, the heterologous protein and a peptide tag is expressed and secreted into the supernatant of the culture;
ii) targeting the peptide tag e.g. by affinity column purification of the supernatant.

Thus in another particular embodiment the method of purifying a heterologous protein comprises
i) culturing the Gram-negative bacterial strain as described herein so that a fusion protein which comprises a delivery signal from a bacterial T3SS effector protein, the heterologous protein, a protease cleavage site between the delivery signal from the bacterial T3SS effector protein and the heterologous protein and a peptide tag is expressed and secreted into the supernatant of the culture;
ii) adding a protease to the supernatant of the culture wherein the protease cleaves the fusion protein so that the heterologous protein is cleaved from the delivery signal from the bacterial T3SS effector protein;
ii) targeting the peptide tag e.g. by affinity column purification of the supernatant.

In the above described particular embodiments the protease can be added to the supernatant of the culture in the form of e.g a purified protease protein or by adding a bacterial strain expressing and secreting a protease to the supernatant of the culture. Further steps may include removal of the protease e.g. via affinity column purification.

In one embodiment the present invention provides the recombinant Gram-negative bacterial strain as described herein for use in medicine.

In one embodiment the present invention provides the recombinant Gram-negative bacterial strain as described herein for use in the delivery of a heterologous protein as a medicament or as a vaccine to a subject. The heterologous protein can be delivered to a subject as a vaccine by contacting the Gram-negative bacterial strain with eukaryotic cells, e.g. with a living animal in vivo so that the heterologous protein is translocated into the living animal which then produces antibodies against the heterologous protein. The antibodies produced can be directly used or be isolated and purified and used in diagnosis, in research use as well as in therapy. The B-cells poducing the antibodies or the therein contained DNA sequence can be used for further production of specific antibodies for use in diagnosis, in research use as well as in therapy

In one embodiment the present invention provides a method for delivering a heterologous protein, wherein the heterologous protein is delivered in vitro into a eukaryotic cell.

In a further embodiment the present invention provides a method for delivering a heterologous protein, wherein the eukaryotic cell is a living animal wherein the living animal is contacted with the Gram-negative bacterial strain in vivo so that a fusion protein is translocated into the living animal. The preferred animal is a mammal, more preferably a human being.

In a further embodiment the present invention provides the use of the recombinant Gram-negative bacterial strain as described supre for High Throughput Screenings of inhibitors for a cellular pathway or event triggered by the translocated heterologous protein(s).

In a further embodiment the present invention provides a library of Gram-negative bacterial strains, wherein the heterologous protein encoded by the second DNA sequence of the expression vector of the Gram-negative bacterial strains is a human or murine protein, preferably a human protein and, wherein each human or murein protein expressed by a Gram-negative bacterial strain is different in amino acid sequence. A possible library could e.g. contain the 560 protein containing Addgene human kinase Orf collection (Addgene No. 1000000014). As cloning vector for expression the above described expression vectors can be used.

In a further embodiment the present invention provides a kit comprising a vector as described herein and a bacterial strain expressing and secreting a protease capable of cleaving the protease cleavage site comprised by the vector. A particular useful vector is a vector for use in combination with the bacterial strain to deliver a desired protein into eukaryotic cells as described above, wherein the vector comprises in the 5′ to 3′ direction:
a promoter;
a first DNA sequence encoding a delivery signal from a bacterial T3SS effector protein, operably linked to said promoter;
a second DNA sequence encoding a heterologous protein fused in frame to the 3′end of said first DNA sequence; and alternatively
a third DNA sequence encoding a protease cleavage site, wherein the third DNA sequence is located between the 3′end of said first DNA sequence and the 5′end of said second DNA sequence.

### EXAMPLES

### Example 1

### A) Materials and Methods

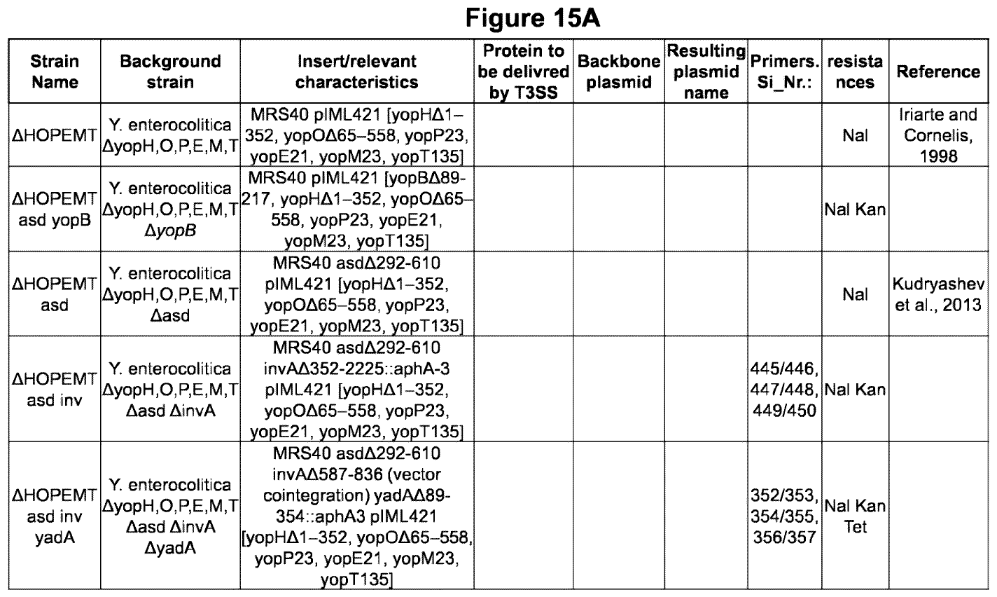

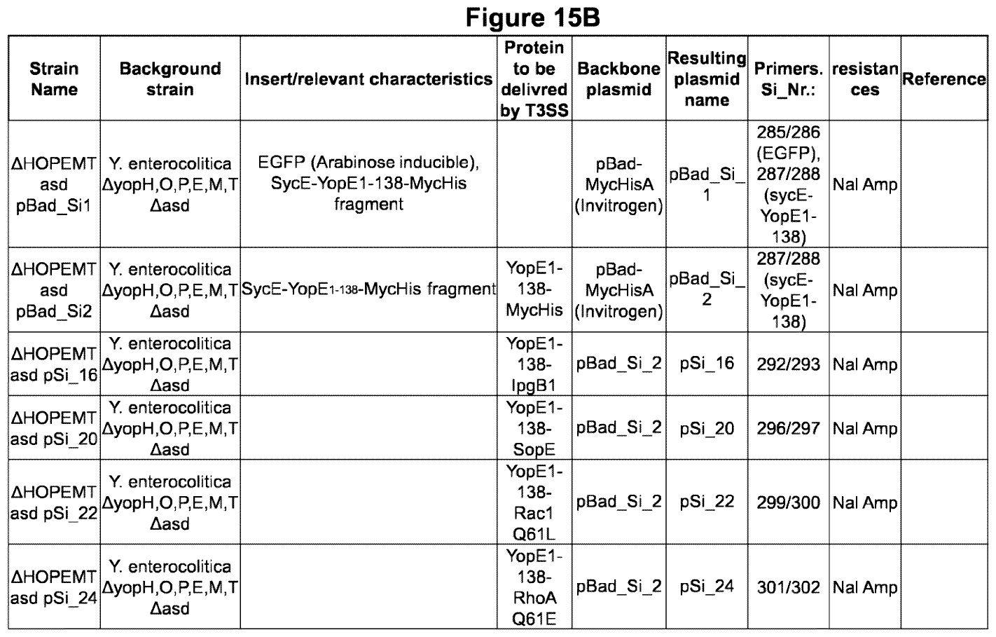

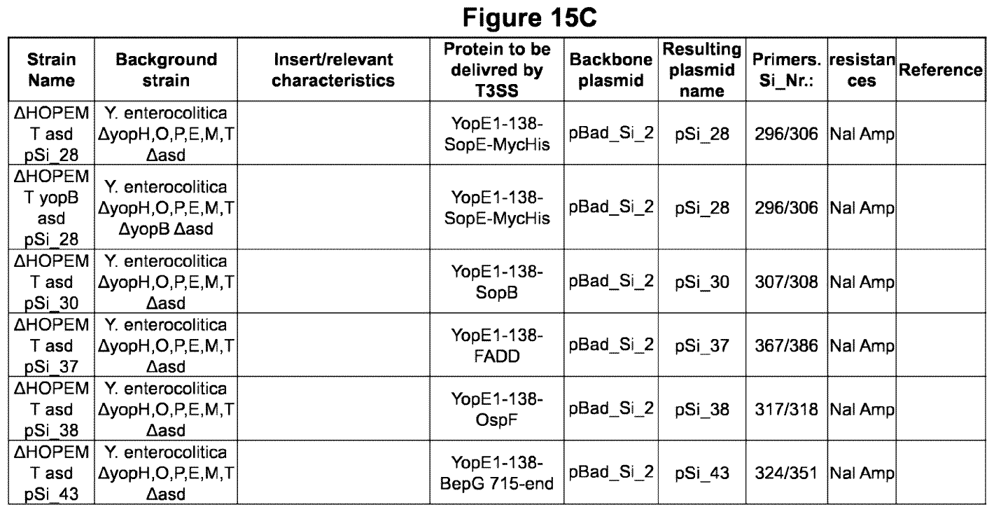

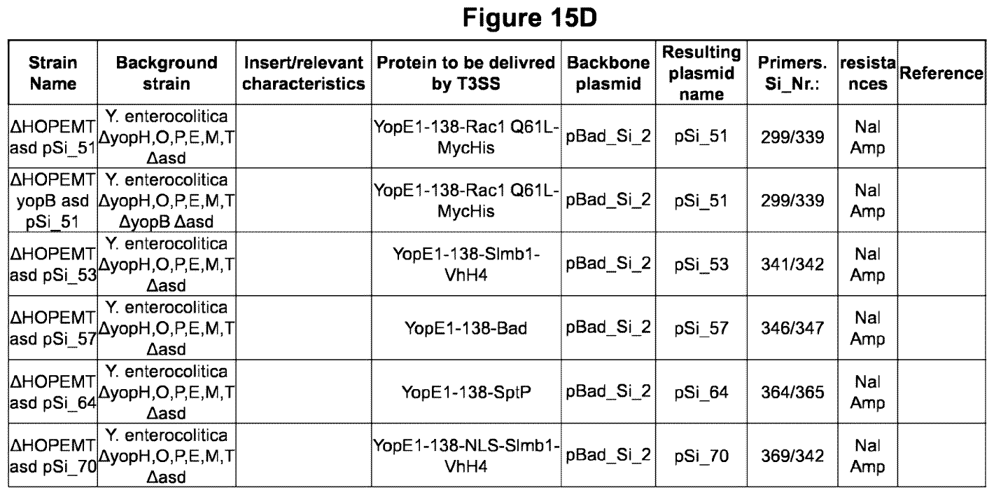

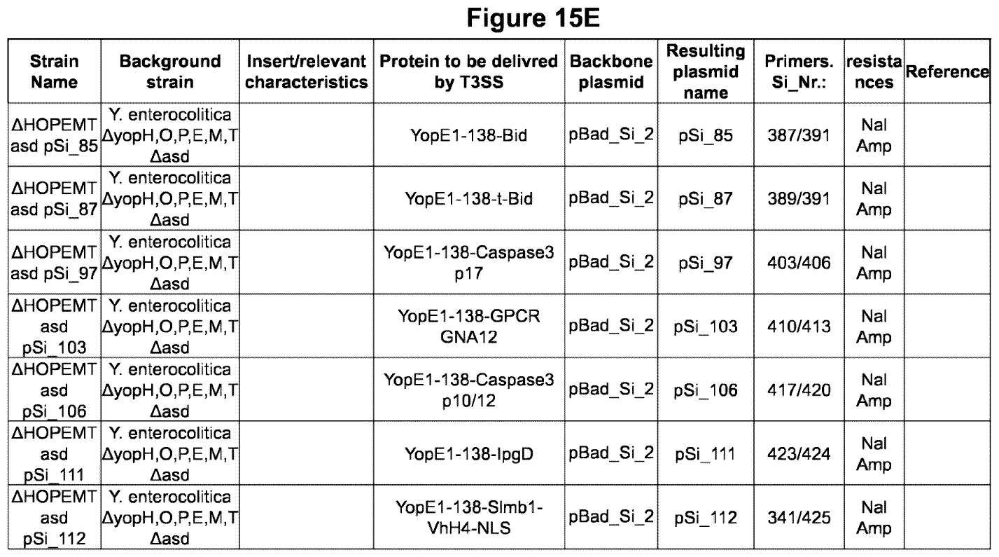

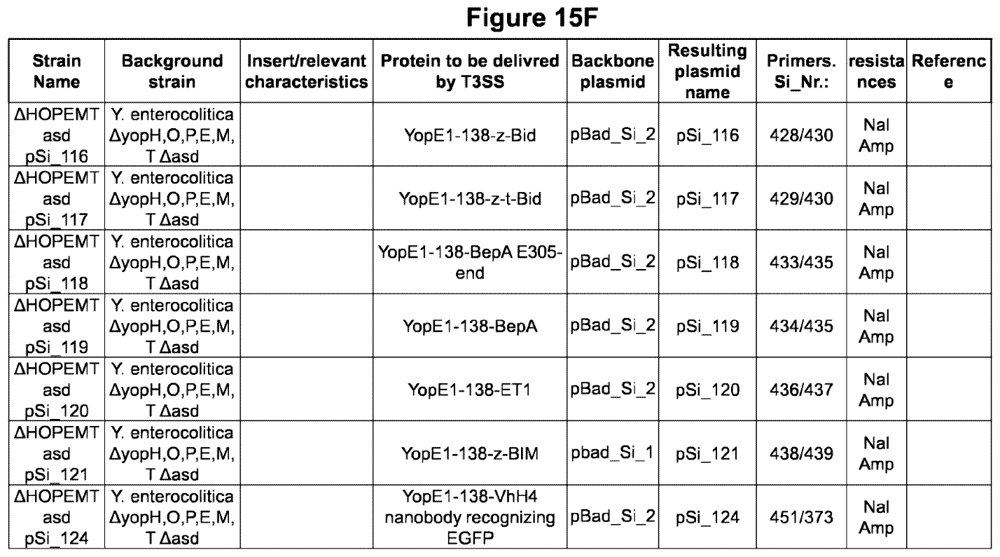

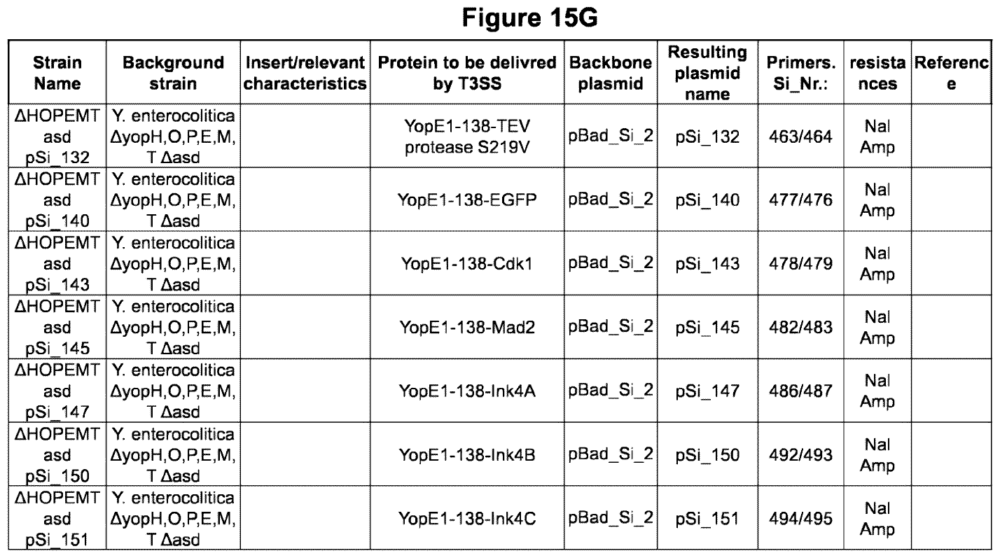

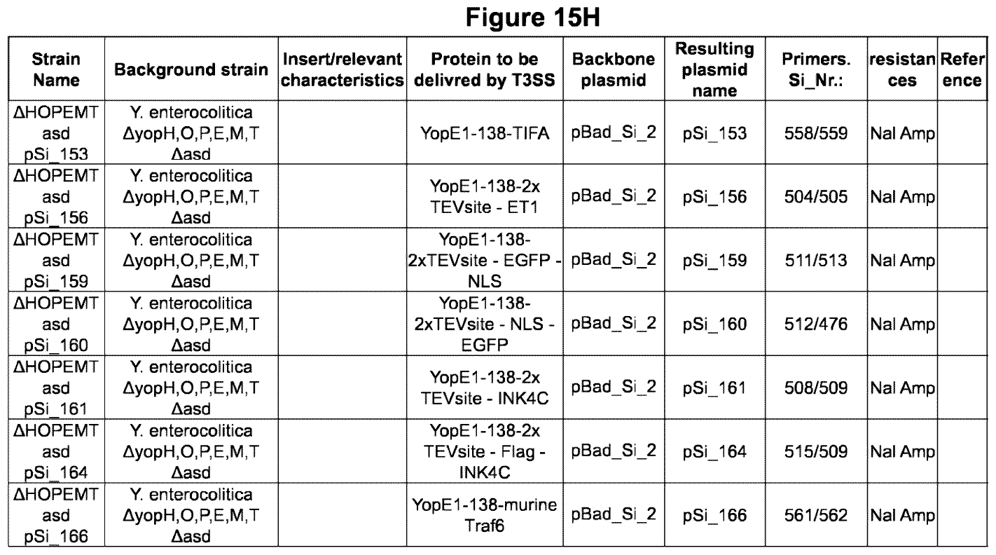

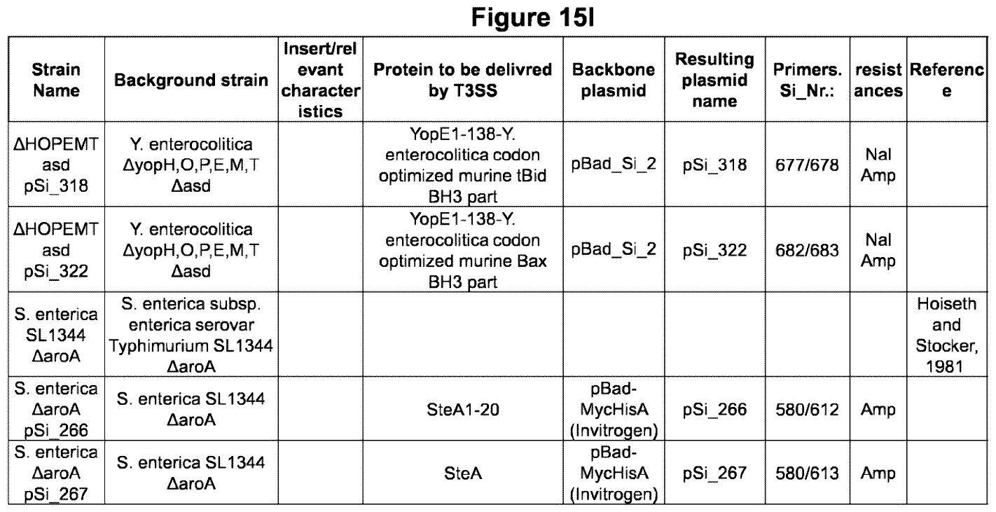

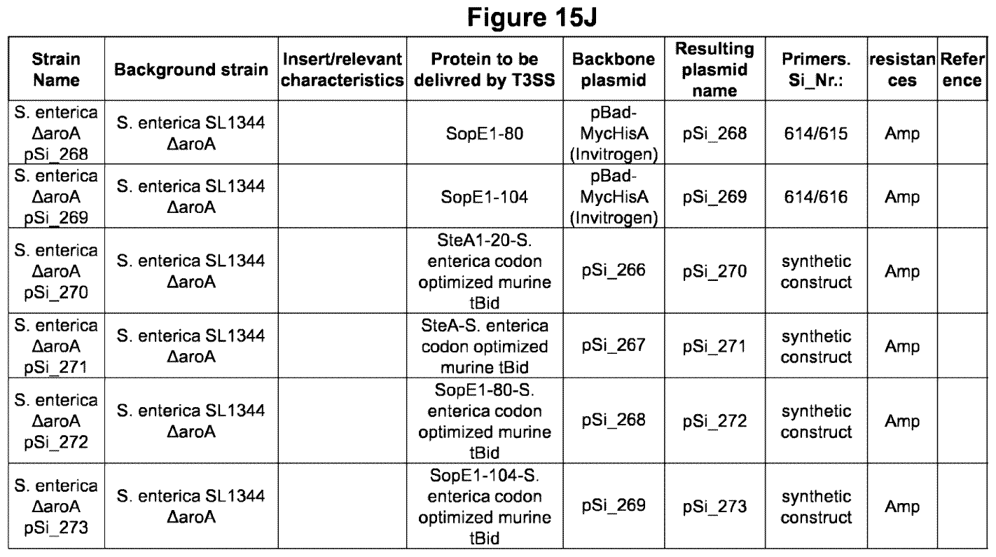

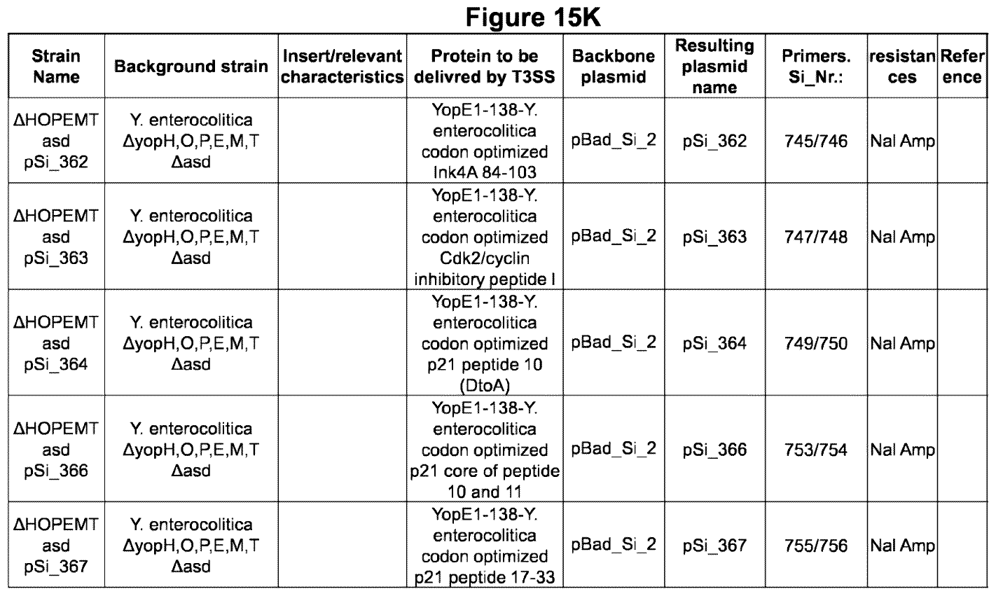

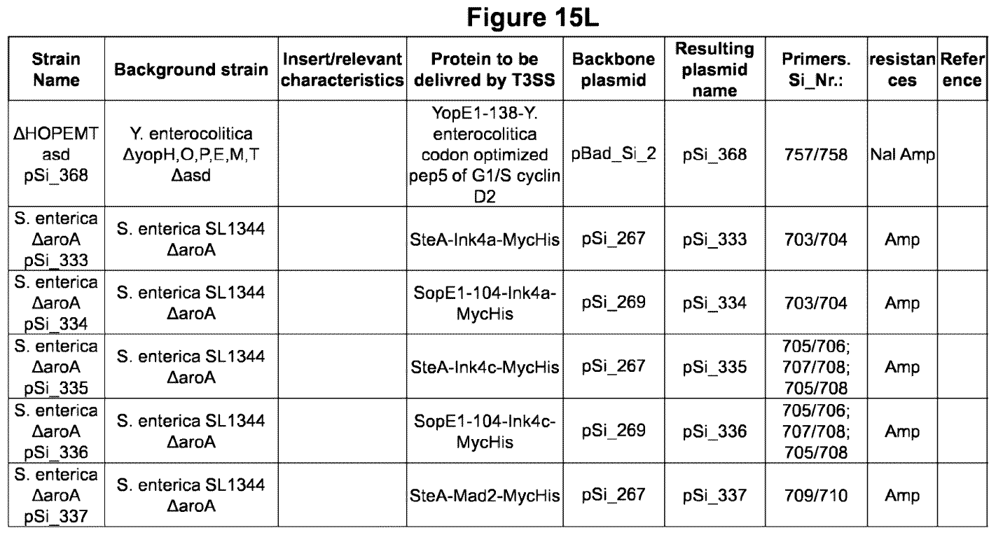

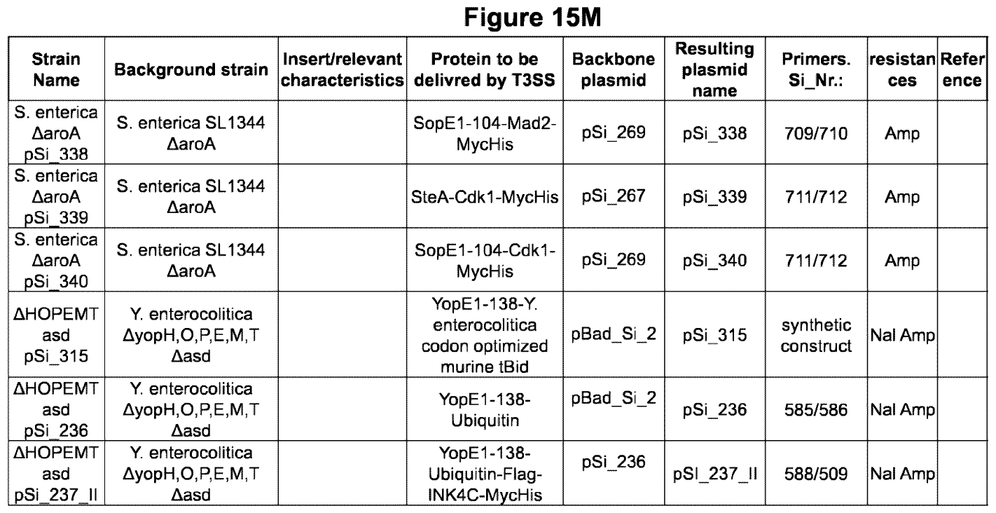

Bacterial strains and growth conditions. The strains used in this study are listed in FIGS. 15A to M. E. coli Top10, used for plasmid purification and cloning, and E. coli Sm10 λ pir, used for conjugation, as well as E. coli BW19610 [46], used to propagate pKNG101, were routinely grown on LB agar plates and in LB broth at 37° C. Ampicillin was used at a concentration of 200 μg/ml (Yersinia) or 100 μg/ml (E. coli) to select for expression vectors. Streptomycin was used at a concentration of 100 μg/ml to select for suicide vectors. Y. enterocolitica MRS40 a non Ampicillin resistant E40-derivate and strains derived thereof were routinely grown on Brain Heart Infusion (BHI; Difco) at RT. To all Y. enterocolitica strains Nalidixic acid was added (35 μg/ml) and all Y. enterocolitica asd strains were additionally supplemented with 100 μg/ml meso-2,6-Diaminopimelic acid (mDAP, Sigma Aldrich). S. enterica SL1344 were routinely grown on LB agar plates and in LB broth at 37° C. Ampicillin was used at a concentration of 100 μg/ml to select for expression vectors in S. enterica.

Genetic manipulations of Y. enterocolitica. Genetic manipulations of Y. enterocolitica has been described [47, 48]. Briefly, mutators for modification or deletion of genes in the pYV plasmids or on the chromosome were constructed by 2-fragment overlapping PCR using purified pYV40 plasmid or genomic DNA as template, leading to 200-250 bp of flanking sequences on both sides of the deleted or modified part of the respective gene. Resulting fragments were cloned in pKNG101 [43] in E. coli BW19610 [46]. Sequence verified plasmids were transformed into E. coli Sm10λ pir, from where plasmids were mobilized into the corresponding Y. enterocolitica strain. Mutants carrying the integrated vector were propagated for severeal generations without selection pressure. Then sucrose was used to select for clones that have lost the vector. Finally mutants were identified by colony PCR.

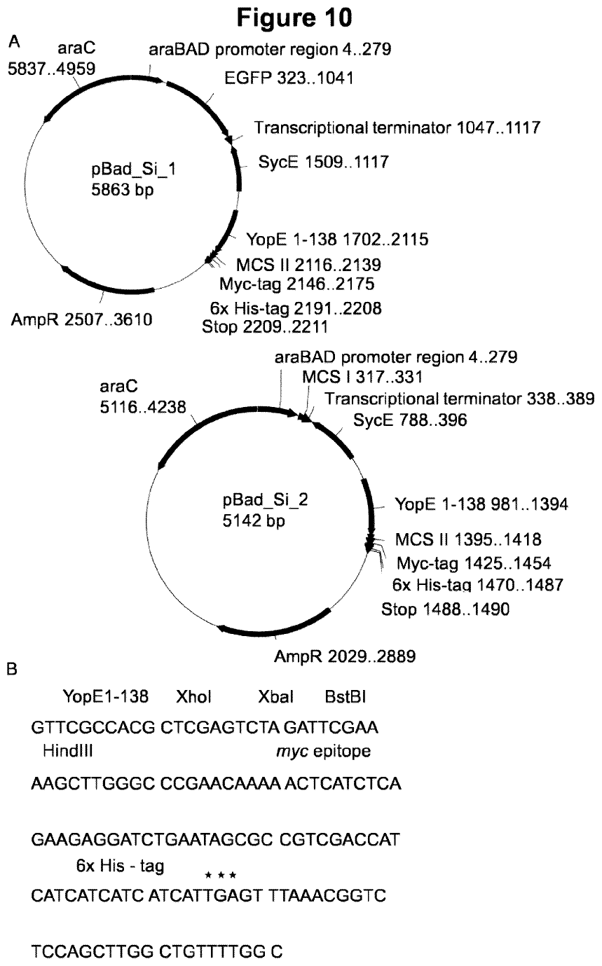

Construction of plasmids. Plasmid pBad_Si2 or pBad_Si1 (FIG. 10) were used for cloning of fusion proteins with the N-terminal 138 amino acids of YopE (SEQ ID No. 2). pBad_Si2 was constructed by cloning of the SycE-YopE₁₋₁₃₈ fragment containing endogenous promoters for YopE and SycE from purified pYV40 into KpnI/HindIII site of pBad-MycHisA (Invitrogen).

Additional modifications include removal of the NcoI/BglII fragment of pBad-MycHisA by digestion, Klenow fragment treatment and religation. A bidirectional transcriptional terminator (BBa_B1006; iGEM foundation) was cloned into KpnI cut and Klenow treated (pBad_Si2) or BglII cut site (pBad_Si1). Further at the 3′ end of YopE₁₋₁₃₈ the following cleavage sites were added: XbaI-XhoI-BstBI-(HindIII) (FIG. 10B). pBad_Si1 is equal to pBad_Si2 but encodes EGFP amplified from pEGFP-C1 (Clontech) in the NcoI/BglII site under the Arabinose inducible promoter. Plasmids pSi_266, pSi_267, pSi_268 and pSi_269 containing the corresponding endogenous promoter and the SteA₁₋₂₀ fragment (pSi_266), the full length SteA sequence (pSi_267), the SopE₁₋₈₁ fragment (pSi_268) or the SopE₁₋₁₀₅ fragment (pSi_269) were amplified from S. enterica SL1344 genomic DNA and cloned into NcoI/KpnI site of pBad-MycHisA (Invitrogen).

Full length genes or fragments thereof were amplified with the specific primers listed in Table I below and cloned as fusions to YopE₁₋₁₃₈ into plasmid pBad_Si2 or in case of z-BIM (SEQ ID No. 21) into pBad_Si1 (see Table II below). For fusion to SteA or SopE, synthetic DNA constructs were cleaved by KpnI/HindII and cloned into pSi_266, pSi_267, pSi_268 or pSi_269 respectively. In case of genes of bacterial species, purified genomic DNA was used as template (S. flexneri M90T, Salmonella enterica subsp. enterica serovar Typhimurium SL1344, Bartonella henselae ATCC 49882). For human genes a universal cDNA library (Clontech) was used if not otherwise stated (FIGS. 15A to M), zebrafish genes were amplified from a cDNA library (a kind gift of M. Affolter). Ligated plasmids were cloned in E. coli Top10. Sequenced plasmids were electroporated into the desired Y. enterocolitica or S. enterica strain using settings as for standard E. coli electroporation.

TABLE I
(Primer Nr. Si_:Sequence)

285: CATACCATGGGAGTGAGCAAGGGCGAG
286: GGAAGATCTttACTTGTACAGCTCGTCCAT
287: CGGGGTACCTCAACTAAATGACCGTGGTG
288: GTTAAAGCTTttcgaatctagactcgagCGTGGCGAACTGGTC
292: CAGTctcgagCAAATTCTAAACAAAATACTTCCAC
293: cagtTTCGAATTAATTTGTATTGCTTTGACGG
296: CAGTctcgagACTAACATAACACTATCCACCCAG
297: GTTAAAGCTTTCAGGAGGCATTCTGAAG
299: CAGTctcgagCAGGCCATCAAGTGTGTG
300: cagtTTCGAATCATTTTCTCTTCCTCTTCTTCA
301: CAGTctcgagGCTGCCATCCGGAA
302: cagtTTCGAATCACAAGACAAGGCACCC
306: GTTAAAGCTTGGAGGCATTCTGAAGatacttatt
307: CAGTctcgagCAAATACAGAGCTTCTATCACTCAG
308: GTTAAAGCTTTCAAGATGTGATTAATGAAGAAATG
317: cagtTTCGAACCCATAAAAAAGCCCTGTC
318: GTTAAAGCTTCTACTCTATCATCAAACGATAAAATGg
324: CAGTctcgagTTCACTCAAGAAACGCAAA
339: cagtTTCGAATTTTCTCTTCCTCTTCTTCAcg
341: cgtaTCTAGAAAAATGATGAAAATGGAGACTG
342: GTTAAAGCTTttaGCTGGAGACGGTGAC
346: CAGTctcgagTTCCAGATCCCAGAGTTTG
347: GTTAAAGCTTTCACTGGGAGGGGG
351: CAGTctcgagctcgagTTATCTACTCATAGAAACTACTTTTGCAG
352: cgcGGATCCtcagtgtctctgcggcatta
353: CATTTATTCCTCCTAGTTAGTCAcagcaactgctgctcctttc
354: gaaaggagcagcagttgctgTGACTAACTAGGAGGAATAAATG
355: cgattcacggattgctttctCATTATTCCCTCCAGGTACTA
356: TAGTACCTGGAGGGAATAATGagaaagcaatccgtgaatcg
357: cgtaTCTAGAcggctttaagtgcgacattc
364: cgtaTCTAGACTAAAGTATGAGGAGAGAAAATTGAA
365: GTTAAAGCTTTCAGCTTGCCGTCGT
367: CGTAtctagaGACCCGTTCCTGGTGC
369: cgtaTCTAGAccccccaagaagaagc
373: GTTAAAGCTTGCTGGAGACGGTGACC
386: CGTAtctagaTCAGGACGCTTCGGAGGTAG
387: CGTAtctagaATGGACTGTGAGGTCAACAA
389: CGTAtctagaGGCAACCGCAGCA
391: GTTAAAGCTTTCAGTCCATCCCATTTCTg
403: CGTAtctagatctggaatatccctggaca
406: GTTAAAGCTTgtctgtctcaatgccacagt
410: CAGTctcgagATGTCCGGGGTGGTg
413: cagtTTCGAATCACTGCAGCATGATGTC
417: CAGTctcgagAGTGGTGTTGATGATGACATG
420: cagtTTCGAATTAGTGATAAAAATAGAGTTCTTTTGTGAG
423: CAGTctcgagATGCACATAACTAATTTGGGATT
424: cagtTTCGAATTATACAAATGACGAATACCCTTT
425: GTTAAAGCTTttacaccttgcgcttcttcttgggcggGCTGGAG
ACGGTGAC
428: CGTAtctagaATGGACTTCAACAGGAACTTT
429: CGTAtctagaGGACATAGTCCACCAGCG
430: GTTAAAGCTTTCAGTTGGATCCGAAAAAC
433: CGTAtctagaGAATTAAAAAAAACACTCATCCCA
434: CGTAtctagaCCAAAGGCAAAAGCAAAAA
435: GTTAAAGCTTTTAGCTAGCCATGGCAAGC
436: CGTAtctagaATGCCCCGCCCC
437: GTTAAAGCTTCTACCCACCGTACTCGTCAAT
438: CGTAtctagaATGTCTGACACGTCCAGAGAG
439: GTTAAAGCTTTCATCTTCTTCGCAGGAAAAAG
445: cgcGGATCCttatgggttctcacagcaaaa
446: CATTTATTCCTCCTAGTTAGTCAaggcaacagccaatcaagag
447: ctcttgattggctgttgcctTGACTAACTAGGAGGAATAAATG
448: ttgattgcagtgacatggtgCATTATTCCCTCCAGGTACTA
449: TAGTACCTGGAGGGAATAATGcaccatgtcactgcaatcaa
450: cgtaTCTAGAtagccgcagatgttggtatg
451: CGTAtctagaGATCAAGTCCAACTGGTGG
463: CAGTctcgaggaaagcttgtttaaggggc
464: cagtTTCGAAttagcgacggcgacg
476: GTTAAAGCTTttACTTGTACAGCTCGTCCAT
477: CGTAtctagaGTGAGCAAGGGCGAG
478: CAGTctcgagATGGAAGATTATACCAAAATAGAGAAA
479: GTTAAAGCTTCTACATCTTCTTAATCTGATTGTCCa
482: CGTAtctagaATGGCGCTGCAGCt
483: GTTAAAGCTTTCAGTCATTGACAGGAATTTTg
486: CGTAtctagaATGGAGCCGGCGGCG
487: GTTAAAGCTTTCAATCGGGGATGTCTg
492: CGTAtctagaATGCGCGAGGAGAACAAGGG
493: GTTAAAGCTTTCAGTCCCCTGTGGCTGTGc
494: CGTAtctagaATGGCCGAGCCTTG
495: GTTAAAGCTTttaTTGAAGATTTGTGGCTCC
504:CGTAtctagaGAAAATCTGTATTTTCAAAGTGAAAATCTGTATTT
TCAAAGTATGCCCCGCCCC
505: GTTAAAGCTTCCCACCGTACTCGTCAATtc
508: CGTAtctagaGAAAATCTGTATTTTCAAAGTGAAAATCTGTATT
TTCAAAGTATGGCCGAGCCTTG
509: GTTAAAGCTTTTGAAGATTTGTGGCTCCc
511: CGTAtctagaGAAAATCTGTATTTTCAAAGTGAAAATCTGTATT
TTCAAAGTGTGAGCAAGGGCGAG
512: CGTAtctagaGAAAATCTGTATTTTCAAAGTGAAAATCTGTATT
TTCAAAGTCCGCCGAAAAAAAAACGTAAAGTTGTGAGCAAGGGCGAG
513: GTTAAAGCTTttAAACTTTACGTTTTTTTTTCGGCGGCTTGTACA
GCTCGTCCAT
515: CGTAtctagaGAAAATCTGTATTTTCAAAGTGAAAATCTGTATT
TTCAAAGTGATTATAAAGATGATGATGATAAAATGGCCGAGCCTTG
558: CGTATCTAGAATGACCAGTTTTGAAGATGC
559: GTTAAAGCTTTCATGACTCATTTTCATCCAT
561: CGTATCTAGAATGAGTCTCTTAAACTGTGAGAACAG
562: GTTAAAGCTTCTACACCCCCGCATCA
580: catgccatggATTTATGGTCATAGATATGACCTC
585: CAGTctcgagATGCAGATCTTCGTCAAGAC
586: GTTAAAGCTTgctagatcgaaACCACCACGTAGACGTAAGAC
588: cagtTTCGAAGATTATAAAGATGATGATGATAAAATGGCCG
AGCCTTG
612: CGGGGTACCatgaggtagatatttcctgataaag
613: CGGGGTACCataattgtccaaatagttatggtagc
614: catgccatggCGGCAAGGCTCCTC
615: cggggtaccTTTATTTGTCAACACTGCCC
616: cggggtaccTGCGGGGTCTTTACTCG
677: TTACTATTCGAAGAAATTATTCATAATATTGCCCGCCATCT
GGCCCAAATTGGTGATGAAATGGATCATTAAGCTTGGAGTA
678: TACTCCAAGCTTAATGATCCATTTCATCACCAATTTGGGCCA
GATGGCGGGCAATATTATGAATAATTTCTTCGAATAGTAA
682: TTACTACTCGAGAAAAAACTGAGCGAATGTCTGCGCCGCATTGG
TGATGAACTGGATAGCTAAGCTTGGAGTA
683: TACTCCAAGCTTAGCTATCCAGTTCATCACCAATGCGGCGCAG
ACATTCGCTCAGTTTTTTCTCGAGTAGTAA

TABLE II
Cloned fusion proteins

| | Pro- | | | | |
|---|---|---|---|---|---|
| | tein | | | | Primer |
| Protein to be | Seq. | | Resulting | | Seq. |
| delivred by | ID. | Backbone | plasmid | Primers. | ID |
| T3SS | No. | plasmid | name | Si_Nr.: | No. |
| YopEl-138- | 3 | pBad- | pBad_Si_1 | 285/286 | 44/45 |
| MycHis | | MycHisA | | (EGFP), | and |
| | | (Invitrogen) | | 287/288 | 46/47 |
| | | | | (sycE- | |
| | | | | YopEl- | |
| | | | | 138) | |
| YopEl-138- | 3 | pBad- | pBad_Si_2 | 287/288 | 46/47 |
| MycHis | | MycHisA | | (sycE- | |
| | | (Invitrogen) | | YopEl- | |
| | | | | 138) | |
| YopEl-138- | 4 | pBad_Si_2 | pSi_16 | 292/293 | 48/49 |
| IpgBl | | | | | |
| YopEl-138- | 5 | pBad_Si_2 | pSi_20 | 296/297 | 50/51 |
| SopE | | | | | |
| YopEl-138- | 26 | pBad_Si_2 | pSi_22 | 299/300 | 52/53 |
| Racl Q61L | | | | | |
| YopEl-138- | 27 | pBad_Si_2 | pSi_24 | 301/302 | 54/55 |
| RhoA Q61E | | | | | |
| YopEl-138- | 135 | pBad_Si_2 | pSi_28 | 296/306 | 50/56 |
| SopE-MycHis | | | | | |
| YopEl-138- | 6 | pBad_Si_2 | pSi_30 | 307/308 | 57/58 |
| SopB | | | | | |
| YopEl-138- | 28 | pBad_Si_2 | pSi_37 | 367/386 | 76/79 |
| FADD | | | | | |
| YopEl-138- | 7 | pBad_Si_2 | pSi_38 | 317/318 | 59/60 |
| OspF | | | | | |
| YopEl-138- | 136 | pBad_Si_2 | pSi_43 | 324/351 | 61/67 |
| BepG 715-end | | | | | |
| YopEl-138- | 137 | pBad_Si_2 | pSi_51 | 299/339 | 52/62 |
| Racl Q61L- | | | | | |
| MycHis | | | | | |
| YopEl-138- | 32 | pBad_Si_2 | pSi_53 | 341/342 | 63/64 |
| Slmbl-VhH4 | | | | | |
| YopEl-138-Bad | 29 | pBad_Si_2 | pSi_57 | 346/347 | 65/66 |
| YopEl-138- | 8 | pBad_Si_2 | pSi_64 | 364/365 | 74/75 |
| SptP | | | | | |
| YopEl-138- | 33 | pBad_Si_2 | pSi_70 | 369/342 | 77/64 |
| NLS-Slmbl- | | | | | |
| VhH4 | | | | | |
| YopEl-138-Bid | 24 | pBad_Si_2 | pSi_85 | 387/391 | 80/82 |
| YopEl-138-t- | 25 | pBad_Si_2 | pSi_87 | 389/391 | 81/82 |
| Bid | | | | | |
| YopEl-138- | 22 | pBad_Si_2 | pSi_97 | 403/406 | 83/84 |
| Caspase3 pl7 | | | | | |
| YopEl-138- | 30 | pBad_Si_2 | pSi_103 | 410/413 | 85/86 |
| GPCR GNA12 | | | | | |
| YopEl-138- | 23 | pBad_Si_2 | pSi_106 | 417/420 | 87/88 |
| Caspase3 | | | | | |
| plO/12 | | | | | |
| YopEl-138- | 9 | pBad_Si_2 | pSi_111 | 423/424 | 89/90 |
| IpgD | | | | | |
| YopEl-138- | 34 | pBad_Si_2 | pSi_112 | 341/425 | 63/91 |
| Slmbl-VhH4- | | | | | |
| NLS | | | | | |
| YopEl-138-z- | 19 | pBad_Si_2 | pSi_116 | 428/430 | 92/94 |
| Bid | | | | | |
| YopEl-138-z-t- | 20 | pBad_Si_2 | pSi_117 | 429/430 | 93/94 |
| Bid | | | | | |
| YopEl-138- | 11 | pBad_Si_2 | pSi_118 | 433/435 | 95/97 |
| BepA E305-end | | | | | |
| YopEl-138- | 10 | pBad_Si_2 | pSi_119 | 434/435 | 96/97 |
| BepA | | | | | |
| YopEl-138- | 36 | pBad_Si_2 | pSi_120 | 436/437 | 98/99 |
| ET1 | | | | | |
| YopEl-138-z- | 21 | pbad_Si_1 | pSi_121 | 438/439 | 100/101 |
| BIM | | | | | |
| YopEl-138- | 31 | pBad_Si_2 | pSi_124 | 451/373 | 108/78 |
| VhH4 nanobody | | | | | |
| recognizing | | | | | |
| EGFP | | | | | |
| YopEl-138- | 42 | pBad_Si_2 | pSi_132 | 463/464 | 109/110 |
| TEV protease | | | | | |
| S219V | | | | | |
| YopEl-138- | 37 | pBad_Si_2 | pSi_140 | 477/476 | 112/111 |
| EGFP | | | | | |
| YopEl-138- | 14 | pBad_Si_2 | pSi_143 | 478/479 | 113/114 |
| Cdkl | | | | | |
| YopEl-138- | 15 | pBad_Si_2 | pSi_145 | 482/483 | 115/116 |
| Mad2 | | | | | |
| YopEl-138- | 16 | pBad_Si_2 | pSi_147 | 486/487 | 117/118 |
| Ink4A | | | | | |
| YopEl-138- | 17 | pBad_Si_2 | pSi_150 | 492/493 | 119/120 |
| Ink4B | | | | | |
| YopEl-138- | 18 | pBad_Si_2 | pSi_151 | 494/495 | 121/122 |
| Ink4C | | | | | |
| YopEl-138- | 13 | pBad_Si_2 | pSi_153 | 558/559 | 131/132 |
| TIFA | | | | | |
| YopEl-138-2x | 41 | pBad_Si_2 | pSi_156 | 504/505 | 123/124 |
| TEVsite-ET1 | | | | | |
| YopEl-138- | 39 | pBad_Si_2 | pSi_159 | 511/513 | 127/129 |
| 2xTEVsite- | | | | | |
| EGFP-NLS | | | | | |
| YopEl-138- | 38 | pBad_Si_2 | pSi_160 | 512/476 | 128/111 |
| 2xTEVsite- | | | | | |
| NLS-EGFP | | | | | |
| YopEl-138-2x | 40 | pBad_Si_2 | pSi_161 | 508/509 | 125/126 |
| TEVsite- | | | | | |
| INK4C | | | | | |
| YopEl-138-2x | 43 | pBad_Si_2 | pSi_164 | 515/509 | 130/126 |
| TEVsite-Flag- | | | | | |
| INK4C | | | | | |
| YopEl-138- | 12 | pBad_Si_2 | pSi_166 | 561/562 | 133/134 |
| murine Traf6 | | | | | |
| YopEl-138-Y. | 138 | pBad_Si_2 | pSi_318 | 677/678 | 148/149 |
| enterocolitica | | | | | |
| codon optimized | | | | | |
| murine tBid | | | | | |
| BH3 part | | | | | |
| YopEl-138-Y. | 139 | pBad_Si_2 | pSi_322 | 682/683 | 150/151 |
| enterocolitica | | | | | |
| codon optimized | | | | | |
| murine Bax BH3 | | | | | |
| part | | | | | |
| SteAl-20 | 140 | pBad- | pSi_266 | 580/612 | 152/153 |
| | | MycHisA | | | |
| | | (Invitrogen) | | | |
| SteA | 141 | pBad- | pSi_267 | 580/613 | 152/154 |
| | | MycHisA | | | |
| | | (Invitrogen) | | | |
| SopEl-81 | 142 | pBad- | pSi_268 | 614/615 | 155/156 |
| | | MycHisA | | | |
| | | (Invitrogen) | | | |
| SopEl-105 | 143 | pBad- | pSi_269 | 614/616 | 155/157 |
| | | MycHisA | | | |
| | | (Invitrogen) | | | |
| SteAl-20-S. | 144 | pSi_266 | pSi_270 | synthetic | / |
| enterica codon | | | | construct | |
| optimized | | | | | |
| murine tBid | | | | | |
| SteA-S. enterica | 145 | pSi_267 | pSi_271 | synthetic | / |
| codon optimized | | | | construct | |
| murine tBid | | | | | |
| SopEl-81-S. | 146 | pSi_268 | pSi_272 | synthetic | / |
| enterica codon | | | | construct | |
| optimized | | | | | |
| murine tBid | | | | | |
| SopEl-105-S. | 147 | pSi_269 | pSi_273 | synthetic | / |
| enterica codon | | | | construct | |
| optimized | | | | | |
| murine tBid | | | | | |
| YopEl-138-Y. | 158 | pBad_Si_2 | pSi_362 | 745/746 | 172/173 |
| enterocolitica | | | | | |
| codon optimized | | | | | |
| Ink4A 84-103 | | | | | |
| YopEl-138-Y. | 159 | pBad_Si_2 | pSi_363 | 747/748 | 174/175 |
| enterocolitica | | | | | |
| codon optimized | | | | | |
| pl07/RBLl | | | | | |
| 657-662 | | | | | |
| (AAA02489.1) | | | | | |
| YopEl-138-Y. | 160 | pBad_Si_2 | pSi_364 | 749/750 | 176/177 |
| enterocolitica | | | | | |
| codon optimized | | | | | |
| p21 141-160 | | | | | |
| (AAH13967.1) | | | | | |
| YopEl-138-Y. | 161 | pBad_Si_2 | pSi_366 | 753/754 | 178/179 |
| enterocolitica | | | | | |
| codon optimized | | | | | |
| p21 145-160 | | | | | |
| (AAH13967.1) | | | | | |
| YopEl-138-Y. | 162 | pBad_Si_2 | pSi_367 | 755/756 | 180/181 |
| enterocolitica | | | | | |
| codon optimized | | | | | |
| p21 17-33 | | | | | |
| (AAH13967.1) | | | | | |
| YopEl-138-Y. | 163 | pBad_Si_2 | pSi_368 | 757/758 | 182/183 |
| enterocolitica | | | | | |
| codon optimized | | | | | |
| cyclin D2 139- | | | | | |
| 147 | | | | | |
| (CAA48493.1) | | | | | |
| SteA-Ink4a- | 164 | pSi_267 | pSi_333 | 703/704 | 184/185 |
| MycHis | | | | | |
| SopEl-105- | 165 | pSi_269 | pSi_334 | 703/704 | 184/185 |
| Ink4a-MycHis | | | | | |
| SteA-Ink4c- | 166 | pSi-267 | pSi_335 | PCR1: | 186/187, |
| MycHis | | | | 705/706 | 188/189 |
| | | | | PCR2: | |
| | | | | 707/708 | |
| | | | | over- | |
| | | | | lapping | |
| | | | | PCR: | |
| | | | | 705/708 | |
| SopEl-105 | 167 | pSi_269 | pSi_336 | PCR1: | 186/187 |
| Ink4c-MycHis | | | | 705/706; | 188/189 |
| | | | | PCR2: | |
| | | | | 707/708; | |
| | | | | over- | |
| | | | | lapping | |
| | | | | PCR: | |
| | | | | 705/708 | |
| SteA-Mad2- | 168 | pSi_267 | pSi_337 | 709/710 | 190/191 |
| MycHis | | | | | |
| SopEl-105- | 169 | pSi_269 | pSi_338 | 709/710 | 190/191 |
| Mad2-MycHis | | | | | |
| SteA-Cdkl- | 170 | pSi_267 | pSi_339 | 711/712 | 192/193 |
| MycHis | | | | | |
| SopEl-105- | 171 | pSi_269 | pSi_340 | 711/712 | 192/193 |
| Cdkl-MycHis | | | | | |
| YopEl-138-Y. | 194 | pBad_Si_2 | pSi_315 | synthetic | / |
| enterocolitica | | | | construct | |
| codon optimized | | | | | |
| murine tBid | | | | | |
| YopEl-138- | 195 | pBad_Si_2 | pSi_236 | 585/586 | 197/198 |
| Ubiquitin | | | | | |
| YopEl-138- | 196 | pSi_236 | pSi_237_II | 588/509 | 199/126 |
| Ubiquitin-Flag- | | | | | |
| INK4C-MycHis | | | | | |

Yop secretion. Induction of the yop regulon was performed by shifting the culture to 37° C. in BHI-Ox (secretion-permissive conditions) [49]. As carbon source glucose was added (4 mg/ml).

Total cell and supernatant fractions were separated by centrifugation at 20 800 g for 10 min at 4° C. The cell pellet was taken as total cell fraction. Proteins in the supernatant were precipitated with trichloroacetic acid 10% (w/v) final for 1 h at 4° C. After centrifugation (20 800 g for 15 min) and removal of the supernatant, the resulting pellet was washed in ice-cold Acetone over-night. The samples were centrifuged again, the supernatant was discarded and the pellet was air-dried and resuspened in 1×SDS loading dye.

Secreted proteins were analysed by SDS-PAGE; in each case, proteins secreted by 3×10⁸ bacteria were loaded per lane. Detection of specific secreted proteins by immunoblotting was performed using 12.5% SDS-PAGE gels. For detection of proteins in total cells, 2×10⁸ bacteria were loaded per lane, if not stated otherwise, and proteins were separated on 12.5% SDS-PAGE gels before detection by immunoblotting.

Immunoblotting was carried out using rat monoclonal antibodies against YopE (MIPA193-13A9; 1:1000, [50]). The antiserum was preabsorbed twice overnight against Y. enterocolitica ΔHOPEMT asd to reduce background staining. Detection was performed with secondary antibodies directed against rat antibodies and conjugated to horseradish peroxidase (1:5000; Southern biotech), before development with ECL chemiluminescent substrate (LumiGlo, KPM).

Cell culture and infections. HeLa Cc12, swiss 3T3 fibroblast cells, 4T1, B16F10 and D2A1were cultured in Dulbecco's modified Eagle's medium (DMEM) supplemented with 10% FCS and 2 mM L-Glutamine (cDMEM). HUVECs were isolated and cultivated as described [51]. Jurkat and 4T cells were cultured in RPMI 1640 supplemented with 10% FCS and 2 mM L-Glutamine. Y. enterocolitica were grown in BHI with additives overnight at RT, diluted in fresh BHI to an OD₆₀₀ of 0.2 and grown for 2 h at RT before a temperature shift to a 37° C. waterbath shaker for further 30 min or for 1 h in case of delivery of EGFP. Finally, the bacteria were collected by centrifugation (6000 rcf, 30 sec) and washed once with DMEM supplemented with 10 mM HEPES and 2 mM L-glutamine. S. enterica were grown in LB with additives overnight at 37° C. and either diluted 1:40 in fresh LB and grown for 2.5 h at 37° C. (Spil T3SS inducting conditions) or the overnight culture was further incubated at 37° C. (Spill T3SS inducing conditions). Finally, the bacteria were collected by centrifugation (6000 rcf, 30 sec) and washed once with DMEM supplemented with 10 mM HEPES and 2 mM L-glutamine. Cells seeded in 96-well (for Immunofluorescence) or 6-well (for Western blotting) plates were infected at indicated MOIs in DMEM supplemented with 10 mM HEPES and 2 mM L-glutamine. After adding bacteria, plates were centrifuged for 1 min at 1750 rpm and placed at 37° C. for indicated time periods. Extracellular bacteria were killed by gentamicin (100 mg/ml) if indicated. In case of immunofluorescence analysis, infection assays were stopped by 4% PFA fixation. For Western blot analysis cells were washed twice with ice-cold PBS and Phospho-safe lysis buffer (Novagen) was added to lyse the cells. After incubation on ice, the cells were centrifuged (16 000 rcf, 25 min, 4° C.). Supernatants were collected and analyzed for total protein content by Bradford BCA assay (Pierce) before SDS PAGE and Western blotting using anti-Phospho-Akt (Ser473 and T308, both Cell Signaling), anti-Actin (Millipore), Anti-Bid (Cell Signaling), anti-Myc (Santa Cruz), anti-p38 (Cell Signaling), anti-phospho-p-38 (Thr180/Tyr182; Cell Signaling), anti-Caspase-3 p17 (Cell Signaling) and anti-Ink4C (Cell Signaling) antibody.

Secretion analysis with S. enterica. For induction of protein secretion by S. enterica, S. enterica were cultivated overnight in LB containing 0.3 M NaCl on an orbital shaker (set to 150 rpm). S. enterica were then diluted 1:50 in fresh LB containing 0.3 M NaCl and grown for 4 h at 37° C. without shaking.

Total cell and supernatant fractions were separated by centrifugation at 20 800 g for 20 min at 4° C. The cell pellet was taken as total cell fraction. Proteins in the supernatant were precipitated with trichloroacetic acid 10% (w/v) final for 1 h at 4° C. After centrifugation (20800 g for 15 min) and removal of the supernatant, the resulting pellet was washed in ice-cold Acetone over-night. The samples were centrifuged again, the supernatant was discarded and the pellet was air-dried and resuspended in 1×SDS loading dye.

Secreted proteins were analysed by SDS-PAGE; in each case, proteins secreted by 3×108 bacteria were loaded per lane. Detection of specific secreted proteins by immunoblotting was performed using 12.5% SDS-PAGE gels. For detection of proteins in total cells, 2×10⁸ bacteria were loaded per lane, if not stated otherwise, and proteins were separated on 12.5% SDS-PAGE gels before detection by immunoblotting. Immunoblotting was carried out using anti-Myc (Santa Cruz) antibody.

Western blotting of T3SS translocated proteins from infected cells. HeLa cells in 6-well plates were infected at an MOI of 100 as described above. In case of coinfection with the TEV protease translocating Y. enterocolitica strain, the OD₆₀₀ of the strains was set and the two bacterial suspensions were mixed in a tube at a ratio of 1:1 (if not otherwise indicated) before addition to the cells. At the end of the infection, the cells were washed twice with ice-cold PBS and collected by scraping in a small volume of ice-cold PBS. After centrifugation (16 000 rcf, 5 min, 4° C.) the pellet was dissolved in 0.002% digitonin supplemented with a protease inhibitor cocktail (Roche complete, Roche). The dissolved pellets were incubated for 5 minutes on ice and then centrifuged (16 000 rcf, 25 min, 4° C.). Supernatants were collected and analyzed for total protein content by Bradford BCA assay (Pierce) before SDS PAGE and Western blotting using an anti-Myc (Santa Cruz, 9E11) or anti-Ink4C (Cell Signaling) antibody.

Immunofluorescence. Cell seeded in 96-well plates (Corning) were infected as described above and after fixation with 4% PFA the cells were washed three times with PBS. The wells were then blocked using 5% goat serum in PBS 0.3% Triton X-100 for 1 h at RT. The primary antibody (anti-Myc, Santa Cruz, 1:100) was diluted in PBS with 1% BSA and 0.3% Triton X-100 and cells were incubated overnight at 4° C. Cells were washed 4 times with PBS before the secondary antibody (AF 488 anti-mouse, life technologies, 1:250) diluted in PBS with 1% BSA and 0.3% Triton X-100 was added. If needed Hoechst DNA staining (life technologies, 1:2500) and/or actin staining (Dy647-Phalloidin, DyeOmics) were included. In some cases only the DNA and/or actin stain was applied directly after washing the PFA off. Cells were incubated for 1 h at RT, washed three times with PBS and analyzed by automated image analysis as described below.

Automated Microscopy and Image Analysis. Images were automatically acquired with an ImageXpress Micro (Molecular devices, Sunnyvale, USA). Quantification of anti-Myc staining intensities was performed using MetaXpress (Molecular devices, Sunnyvale, USA). Regions within cells excluding nuclear regions and regions containing bacteria were manually chosen (circles with an area of 40 pixels) and average intensity was recorded.

TNFα stimulation and Western blotting of phospho-p38. HeLa cells seeded in 6-well plates were infected with an MOI of 100 as described above. 30 min p.i Gentamicin was added and 45 min p.i. TNFa was added (10 ng/ml). 1 h 15 min p.i. cells were washed twice with ice-cold PBS and Phospho-safe lysis buffer (Novagen) was added to lyse the cells. After incubation on ice, the cells were centrifuged (16 000 rcf, 25 min, 4° C.). Supernatants were collected and analyzed for total protein content by Bradford BCA assay (Pierce) before SDS PAGE and Western blotting using an anti-Phospho-p38, total p38 antibodies (Cell Signaling) and anti-Actin antibody (Millipore).

cAMP level determination of infected HeLa cells. HeLa cells seeded in 96-well plates were infected as described above. 30 min before the infection cDMEM was changed to DMEM supplemented with 10 mM HEPES and 2 mM L-glutamine and 100 μM 3-Isobutyl-1-methylxanthin (IBMX, Sigma Aldrich). 60 min p.i. Gentamicin was added and cells were further incubated at 37° C. for another 90 min. Determination of cAMP was performed using a competitive ELISA according to the manufacturers instructions (Amersham, cAMP Biotrak, RPN225). As a positive control indicated amount of cholera toxin (C8052, Sigma Aldrich) was added for 1 h to cells in DMEM supplemented with 10 mM HEPES and 2 mM L-glutamine and 100 uM IBMX.

Zebrafish embryo infections, imaging and automated image quantification. All animal experiments were performed according to approved guidelines. Zebrafish were maintained at standard conditions [52]. Embryos were staged by hours postfertilization (hpf) at 28.5° C. [53].The following zebrafish lines were used in this study: wild type fish (AB/EK and EK/TL). Infection protocol followed guidelines given in [54]. 12 hpf embryos were maintained in E3medium containing 0.2 mM N-phenylthiourea (PTU) to prevent pigment formation. 2 days postfertilization (dpf) embryos were anesthetized by 0.2 mg/ml Tricaine and aligned on 1% agar plates in E3 using a hair loop tool [54]. Y. enterocolitica were grown in BHI supplemented with 0.4% Arabinose and antibiotics and mDap overnight at RT, diluted in fresh BHI with 0.5% Arabinose and other additives to an OD₆₀₀ of 0.2 and grown for 2 h at RT before a temperature shift to a 37° C. waterbath shaker for further 45 min. Finally, the bacteria were collected by centrifugation (6000 rcf, 30 sec) and washed once with PBS. The OD₆₀₀ was set to 2 in PBS containing mDAP. 1-2 nL of this suspension were injected into the hindbrain of aligned zebrafish embryos using an Femtojet Microinjector (Eppendorf) using Femtotips II (Eppendorf), where the tip of the needle had been broken off with fine tweezers. The injection time was set to 0.2 s and the compensation pressure to 15 hPa (Eppendorf, Femtojet) and the injection pressure was adjusted between 600 and 800 hPa. Drop size and thus the inoculum was checked by microscopy and by control plating. Following microinjection the fish were collected in E3 containing Tricaine and PTU and incubated for 30 min at 37° C. and incubated for further 5 h at 28° C. A fluorescence binocular (Leica) was used to observe bacterial EGFP fluorescence 1 h post infection in zebrafish hindbrains, and embryos that are not properly injected were discarded. At the end of the infection, fish were fixed with 2% ice-cold PFA for 1 h on ice and further with fresh ice-cold PFA overnight at 4° C. Antibody staining was performed as described previously [55, 56]. Biefly, embryos were washed 4 times with PBS 0.1% Tween for 5 min each wash and permeabilized with PBS-T+0.5% Triton X-100 for 30 min at RT. Embryos were blocked in blocking solution (PBS 0.1% Tween 0.1% TritonX-100 5% goat serum and 1% BSA) at 4° C. overnight. Antibody (Cleaved Caspase-3 (Asp175), Cell Signaling) was diluted 1:100 in blocking solution and incubated under shaking at 4° C. in the dark. Fish were washed 7 times with PBS 0.1% Tween for 30 min before the secondary antibody (goat anti-rabbit AF647, Invitrogen, 1:500) diluted in blocking solution was added and incubated at 4° C. overnight. Larvae were washed with PBS 0.1% Tween four times 30 min at 4° C. and once overnight and further washed 3-4 times. Images were taken with Leica TCS SP5 confocal microscope using a 40× water immersion objective. Images were analyzed using Imaris (Bitplane) and Image J software (http://imagej.nih.gov/ij/).

Image analysis (on n=14 for pBad_Si2 or n=19 for z-BIM) was performed via CellProfiler [57] on maximum intensity z projections of recorded z-stack images. Briefly, bacteria were detected via the GFP channel. Around each area of a bacterial spot a circle with a radius of 10 pixels was created. Overlapping regions were separated equally among the connecting members. In those areas closely surrounding bacteria, the Caspase 3 p17 staining intensity was measured.

Sample Preparation for Phosphoproteomics. For each condition, two 6-well plates of HeLa CCL-2 cells were grown to confluency. Cells were infected for 30 min as described above. At the indicated time-points, the plates were put on ice and washed twice with ice-cold PBS. Samples were then collected in urea solution [8 M Urea (AppliChem), 0.1 M Ammoniumbicarbonate (Sigma), 0.1% RapiGest (Waters), 1×PhosSTOP (Roche)]. The samples were briefly vortexed, sonicated at 4° C. (Hielscher), shaked for 5 min on a thermomixer (Eppendorf) and centrifuged for 20 min at 4° C. and 16'000 g. Supernatants were collected and stored at −80° C. for further processing. BCA Protein Assay (Pierce) was used to measure protein concentration.

Phosphopeptide Enrichment. Disulfide bonds were reduced with tris (2-carboxyethyl) phosphine at a final concentration of 10 mM at 37° C. for 1 h. Free thiols were alkylated with 20 mM iodoacetamide (Sigma) at room temperature for 30 min in the dark. The excess of iodoacetamide was quenched with N-acetyl cysteine at a final concentration of 25 mM for 10 min at room temperature. Lys-C endopeptidase (Wako) was added to a final enzyme/protein ratio of 1:200(w/w) and incubated for 4 h at 37° C. The solution was subsequently diluted with 0.1 M ammoniumbicarbonate (Sigma) to a final concentration below 2 M urea and digested overnight at 37° C. with sequencing-grade modified trypsin (Promega) at a protein-to-enzyme ratio of 50:1. Peptides were desalted on a C18 Sep-Pak cartridge (Waters) and dried under vacuum. Phosphopeptides were isolated from 2 mg of total peptide mass with TiO₂ as described previously [58]. Briefly, dried peptides were dissolved in an 80% acetonitrile (ACN)−2.5% trifluoroacetic acid (TFA) solution saturated with phthalic acid. Peptides were added to the same amount of equilibrated TiO₂ (5-μm bead size, GL Sciences) in a blocked Mobicol spin column (MoBiTec) that was incubated for 30 min with end-over-end rotation. The column was washed twice with the saturated phthalic acid solution, twice with 80% ACN and 0.1% TFA, and finally twice with 0.1% TFA. The peptides were eluted with a 0.3 M NH₄OH solution. The pH of the eluates was adjusted to be below 2.5 with 5% TFA solution and 2 M HCl. Phosphopeptides were again desalted with microspin C18 cartridges (Harvard Apparatus).

LC-MS/MS analysis. Chromatographic separation of peptides was carried out using an EASY nano-LC system (Thermo Fisher Scientific), equipped with a heated RP-HPLC column (75 μm×45 cm) packed in-house with 1.9 μm C18 resin (Reprosil-AQ Pur, Dr. Maisch). Aliquots of 1 μg total phosphopeptide sample were analyzed per LC-MS/MS run using a linear gradient ranging from 98% solvent A (0.15% formic acid) and 2% solvent B (98% acetonitrile, 2% water, 0.15% formic acid) to 30% solvent B over 120 minutes at a flow rate of 200 nl/min. Mass spectrometry analysis was performed on a dual pressure LTQ-Orbitrap mass spectrometer equipped with a nanoelectrospray ion source (both Thermo Fisher Scientific). Each MS1 scan (acquired in the Orbitrap) was followed by collision-induced dissociation (CID, acquired in the LTQ) of the 20 most abundant precursor ions with dynamic exclusion for 30 seconds. For phosphopeptide analysis the 10 most abundant precursor ions were subjected to CID with enabled multistage activation. Total cycle time was approximately 2 s. For MS1, 10⁶ ions were accumulated in the Orbitrap cell over a maximum time of 300 ms and scanned at a resolution of 60,000 FWHM (at 400 m/z). MS2 scans were acquired using the normal scan mode, a target setting of 10⁴ ions, and accumulation time of 25 ms. Singly charged ions and ions with unassigned charge state were excluded from triggering MS2events. The normalized collision energy was set to 32%, and one microscan was acquired for each spectrum.

Label-free quantification and database searching. The acquired raw-files were imported into the Progenesis software tool (Nonlinear Dynamics, Version 4.0) for label-free quantification using the default parameters. MS2 spectra were exported directly from Progenesis in mgf format and searched using the MASCOT algorithm (Matrix Science, Version 2.4) against a decoy database [59] containing normal and reverse sequences of the predicted SwissProt entries of Homo sapiens (www.ebi.ac.uk, release date 16 May 2012) and commonly observed contaminants (in total 41,250 sequences) generated using the SequenceReverser tool from the MaxQuant software (Version 1.0.13.13). To identify proteins originating from Y. enterocolitica, non phosphopeptide enriched samples were searched against the same database above including predicted SwissProt entries of Y. enterocolitica (www.ebi.ac.uk, release date 15 Aug. 2013) The precursor ion tolerance was set to 10 ppm and fragment ion tolerance was set to 0.6 Da. The search criteria were set as follows: full tryptic specificity was required (cleavage after lysine or arginine residues unless followed by proline), 2 missed cleavages were allowed, carbamidomethylation (C) was set as fixed modification and phosphorylation (S,T,Y) or oxidation (M) as a variable modification for TiO2 enriched or not enriched samples, respectively. Finally, the database search results were exported as an xml-file and imported back to the Progenesis software for MS1 feature assignment. For phosphopeptide quantification, a csv-file containing the MS1 peak abundances of all detected features was exported and for not enriched samples, a csv-file containing all protein measurements based on the summed feature intensities of all identified peptides per protein was created. Importantly, the Progenesis software was set that proteins identified by similar sets of peptides are grouped together and that only non-conflicting peptides with specific sequences for single proteins in the database were employed for protein quantification. Both files were further processed using the in-house developed SafeQuant v1.0 R script (unpublished data, available at https://github.com/eahrne/SafeQuant/). In brief, the software sets the identification level False Discovery Rate to 1% (based on the number of decoy protein sequence database hits) and normalizes the identified MS1 peak abundances (Extracted Ion Chromatogram, XIC) across all samples, i.e. the summed XIC of all confidently identified peptide features is scaled to be equal for all LC-MS runs. Next, all quantified phosphopeptides/proteins are assigned an abundance ratio for each time point, based on the median XIC per time point. The statistical significance of each ratio is given by its q-value (False Discovery Rate adjusted p-values), obtained by calculating modified t-statistic p-values and adjusting for multiple testing [61]. The location of the phosphorylated residues was automatically assigned by MASCOT (score>10). All annotated spectra together with the MS raw files and search parameters employed, will be deposited to the ProteomeXchange Consortium (http:/proteomecentral.protcomexchange.org) via the PRIDE partner repository [62].

Sequence alignment was performed using EMBL-EBI web based ClustalW2 multiple sequence alignment tool at http://www.ebi.ac.uk/Tools/msa/clustalw2/.

### B) Results

### A Protein Delivery System Based on Type 3 Secretion of YopE Fusion Proteins

While the very N-terminus of the Y. enterocolitica T3SS effector YopE (SEQ ID No. 1) contains the secretion signal sufficient to translocate heterologous proteins [10], the chaperone-binding site (CBS) for its chaperone (SycE) is not included [63]. We selected the N-terminal 138 amino acids of YopE (SEQ ID No. 2) to be fused to proteins to be delivered, as this had been shown to give best results for translocation of other heterologous T3S substrates [38]. As these N-terminal 138 amino acids of YopE contain the CBS, we further decided to coexpress SycE. The SycE-YopE₁₋₁₃₈ fragment cloned from purified Y. enterocolitica pYV40 virulence plasmid contains the endogenous promoters of YopE and of its chaperone SycE (FIG. 10). Therfore, SycE and any YopE₁₋₁₃₈ fusion protein are induced by a rapid temperature shift from growth at RT to 37° C. Culture time at 37° C. will affect fusion protein amount present in bacteria. A multiple cloning site (MCS) was added at the 3′ end of YopE₁₋₁₃₈ (FIG. 10B) followed by a Myc and a 6×His tag and a Stop codon.

The background strain was carefully selected. First, to limit the translocation of endogenous effectors, we used a Y. enterocolitica strain that was deleted for all known effectors, Yop H, O, P, E, M and T (named ΔHOPEMT) [64]. In addition, we used an auxotroph mutant that cannot grow in absence of exogenous meso-2,6-diaminopimelic acid [65]. This strain was deleted for the aspartate-beta-semialdehyde dehydrogenase gene (Δasd), and classified as biosafety level 1 by the Swiss safety agency (amendment to A010088/2). In addition, we deleted the adhesion proteins YadA and/or InvA to offer a larger choice of background strains. While the use of the yadA or yadA/invA strains reduce the background signalling induced [66], the delivered protein amount is affected as well [67].

### Characterization of YopE Fusion Protein Delivery Into Eukaryotic Cells

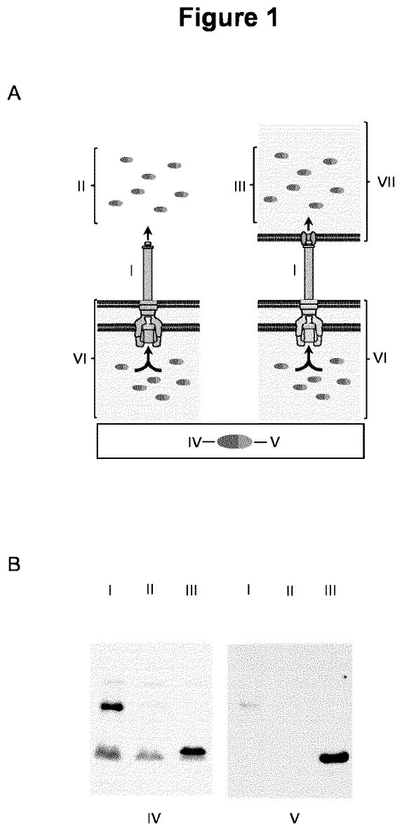

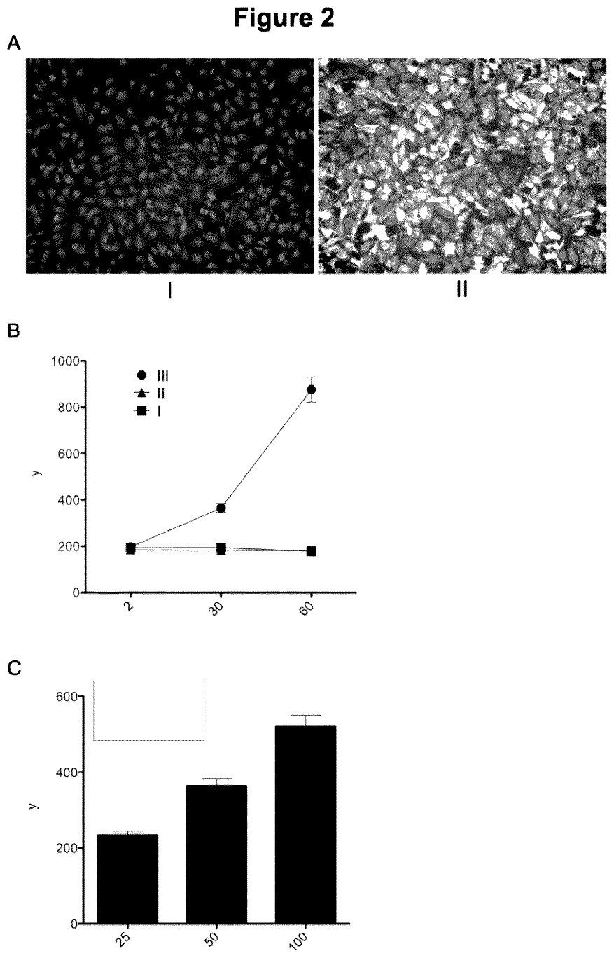

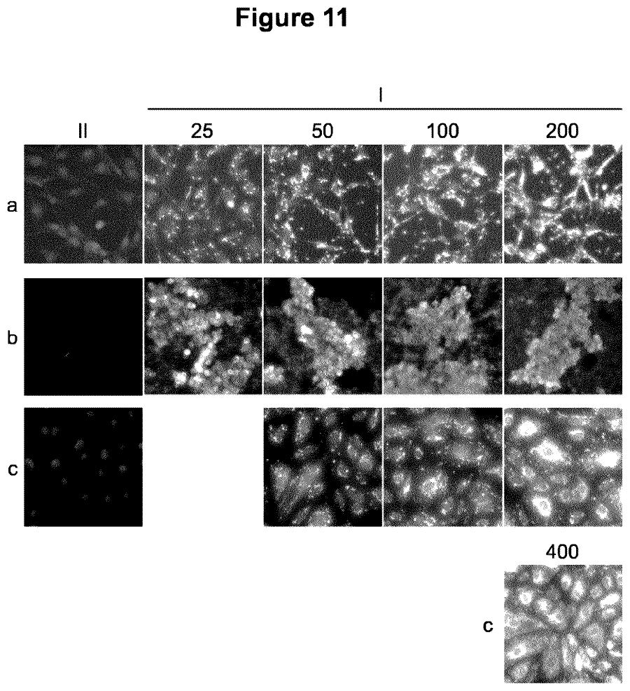

In an in-vitro secretion assay (see FIG. 1A), protein secretion into the surrounding liquid is artificially induced. After TCA based protein precipitation, Western blot analysis with anti-YopE antibody was used to determine protein amounts secreted (FIG. 1B). While a wt strain secreted full length YopE, the ΔHOPEMT asd strains did not. Upon presence of YopE₁₋₁₃₈-Myc-His (further termed YopE₁₋₁₃₈-Myc; SEQ_ID_No._3) a smaller YopE band became visible (FIG. 1B). Hence, the YopE₁₋₁₃₈ fragment is well secreted in the set up described here. To analyze homogeneity of protein translocation into eukaryotic cells, we infected HeLa cells with the YopE₁₋₁₃₈-Myc encoding strain and stained the Myc tag by IF (FIGS. 2A and B). While in the beginning only the bacteria were stained, at 30 min post infection (p.i.) cell outlines start to be visible, which is enhanced upon increased infection time (FIG. 2B). This trend is well reflected by the Myc tag staining intensity inside HeLa cells (FIGS. 2A and B). The YopE₁₋₁₃₈-Myc can be detected everywhere in the cells (FIG. 2A), except in the nuclei [68]. Remarkably, most if not all cells were reached by this approach in a comparable way. As Y. enterocolitica is known to infect many different cell types [69], we followed YopE₁₋₁₃₈-Myc delivery into various cell lines. The same homogenous anti-Myc IF staining was observed in infected murine fibroblasts, Jurkat cells and HUVECs (FIG. 11). Even more, tuning the MOI up or down allows modulating the protein amount delivered (FIG. 2C), while still most of the cells remain targeted. A low bacterial number will not result in few cells with lots of delivered protein but rather with most cells containing a low amount of delivered protein (FIG. 2C).

### Redirection of T3SS Delivered Proteins to the Nucleus

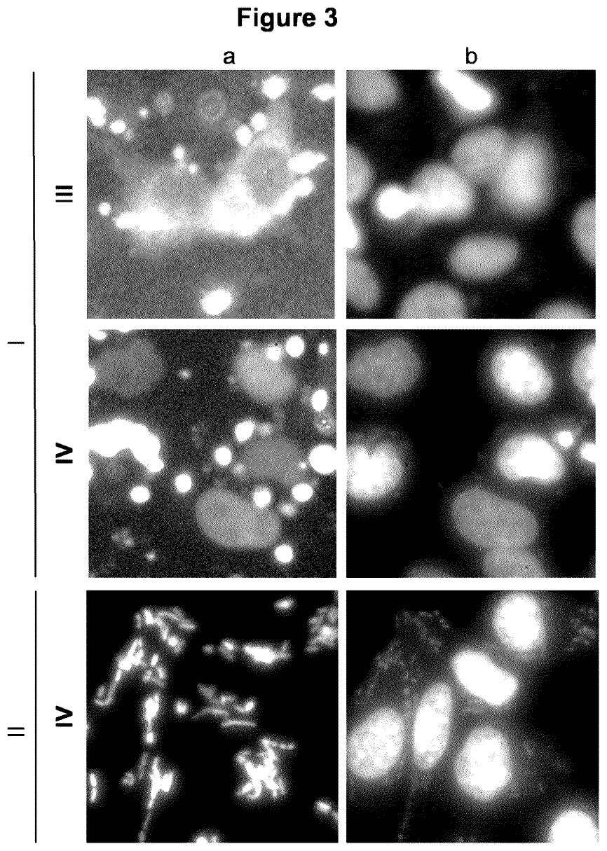

As YopE itself localized to the cytoplasm (FIG. 2A), it is of special interest to test if the YopE₁₋₁₃₈ fragment hampers localization of nuclear fusion proteins. We therefore added the SV40 NLS to the C-terminus (and N-terminus, similar results) of YopE₁₋₁₃₈-EGFP (SEQ ID No. 39 and SEQ ID No. 38, respectively). While YopE₁₋₁₃₈-EGFP (SEQ ID No. 37) led to a weak cytoplasmic staining, YopE₁₋₁₃₈-EGFP-NLS gave rise to a stronger nuclear EGFP signal in HeLa cells infected (FIG. 3). This indicates that the YopE₁₋₁₃₈ fragment is compatible with the use of an NLS. While mCherry had already been used in plant pathogens [70], this

represents a successful delivery of a GFP-like protein via human or animal pathogenic bacteria encoding a T3SS. This validates the SycE and YopE₁₋₁₃₈ dependent strategy to be very promising for delivery of many proteins of choice.

### Removal of the YopE₁₋₁₃₈ Appendage After Translocation of the Fusion Protein to the Eukaryotic Cell

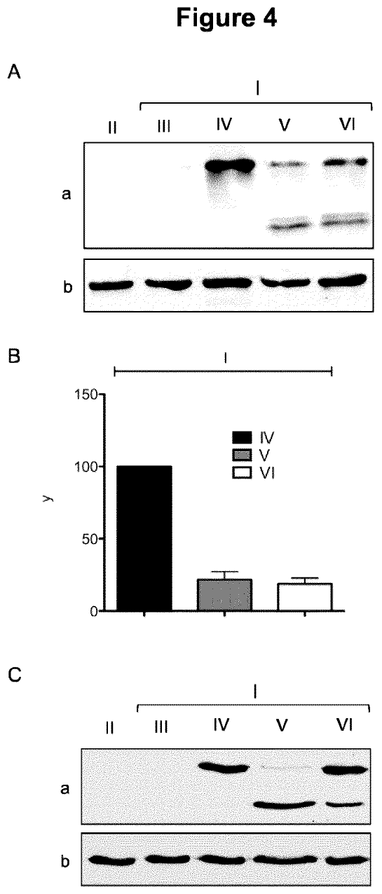

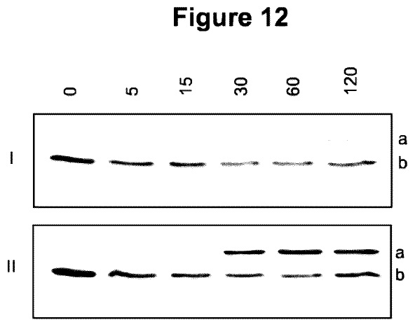

While for bacterial delivery the YopE₁₋₁₃₈ fragment is of great benefit, it might hamper the fusion proteins function and/or localization. Therefore, its removal after protein delivery would be optimal. To this end, we introduced two TEV cleavage sites (ENLYFQS) [71-73] in between YopE₁₋₁₃₈ and a fusion partner (the transcriptional regulator ET1-Myc (SEQ ID No. 36 and 41) and human INK4C (SEQ ID No. 40 and SEQ ID No. 43)). To keep the advantages of the presented method, we further fused the TEV protease (S219V variant; [75]) to YopE₁₋₁₃₈ (SEQ ID No. 42) in another Y. enterocolitica strain. HeLa cells were infected with both strains at once. To allow analysis of the translocated fraction of proteins only, infected HeLa cells were lysed at 2 h p.i. (FIG. 4) with Digitonin, which is known not to lyse the bacteria ([76]; see FIG. 12 for control). Western blot analysis revealed the presence of the YopE₁₋₁₃₈-2xTEV-cleavage-site-ETI-Myc or YopE₁₋₁₃₈-2xTEV-cleavage-site-Flag-INK4C-Myc only when cells had been infected with the corresponding strain (FIGS. 4A and C). Upon overnight digestion of this cell-lysate with purified TEV protease, a shifted band could be observed (FIGS. 4A and C). This band corresponds to ET1-Myc (FIG. 4C) or Flag-INK4C (FIG. 4A) with the N-terminal remnants of the TEV cleavage site, most likely only one Serine. Upon coinfection of cells with the strain delivering the TEV protease, the same cleaved ET1-Myc or Flag-INK4C fragment became visible, indicating that the TEV protease delivered via T3SS is functional and that single cells had been infected by both bacterial strains (FIGS. 4A and C). While cleavage is not complete, the majority of translocated protein is cleaved already 2 h post infection and even over-night digestion with purified TEV protease did not yield better cleavage rates (FIG. 4B). As reported, TEV protease dependent cleavage might need optimization dependent on the fusion protein [77, 78]. TEV protease dependent removal of the YopE₁₋₁₃₈ appendage after translocation hence provides for the first time a T3SS protein delivery of almost native heterologous proteins, changing the amino acid composition by only one N-terminal amino acid.

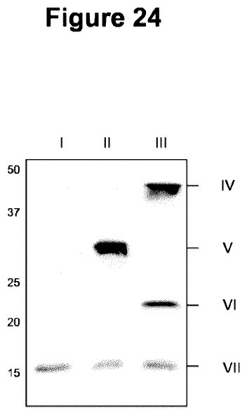

An alternative approach to the TEV protease dependent cleavage of the YopE fragment consisted in incorporating Ubiquitin into the fusion protein of interest. Indeed, Ubiquitin is processed at its C-terminus by a group of endogenous Ubiquitin-specific C-terminal proteases (Deubiquitinating enzymes, DUBs). As the cleavage is supposed to happen at the very C-terminus of Ubiquitin (after G76), the protein of interest should be free of additional amino acid sequence. This method was tested on the YopE1-138-Ubiquitin-Flag-INK4C-MycHis fusion protein. In control cells infected by YopE1-138-Flag-INK4C-MycHis-expressing bacteria, a band corresponding to YopE1-138-Flag-INK4C-MycHis was found, indicative of efficient translocation of the fusion protein (FIG. 24). When cells were infected for 1 h with YopE1-138-Ubiquitin-Flag-INK4C-MycHis-expressing bacteria, an additional band corresponding to the size of Flag-INK4C-MycHis was visible, indicating that part of the fusion protein was cleaved. This result shows that the introduction of Ubiquitin into the fusion protein enables to cleave off the YopE1-138 fragment without a need for an exogenous protease.

### Translocation of Type III and Type IV Bacterial Effectors

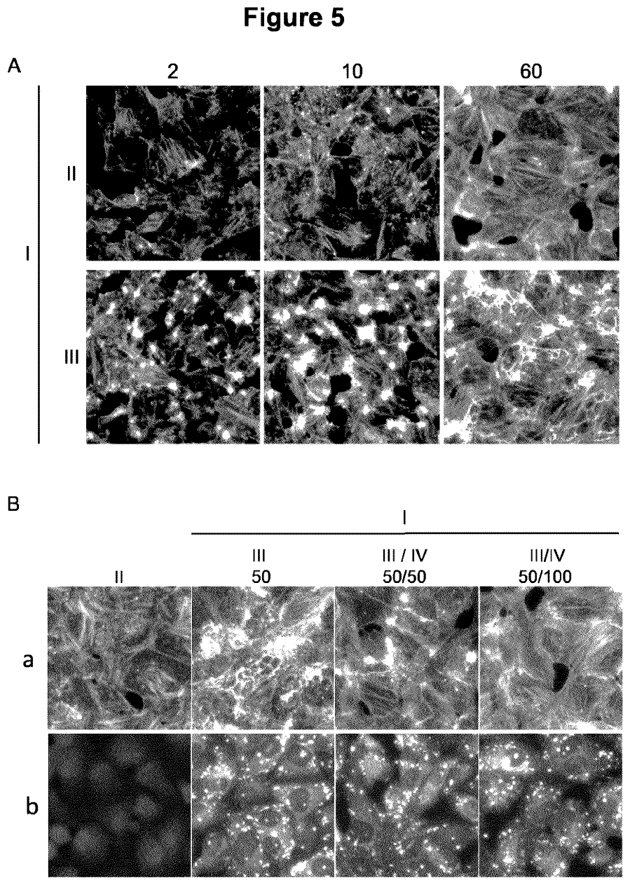

SopE from Salmonella enterica is a well-characterized guanine nucleotide exchange factor (GEF) that interacts with Cdc42, promoting actin cytoskeletal remodeling [79]. Whereas the translocation of YopE₁₋₁₃₈-Myc into HeLa cells has no effect, translocated YopE₁₋₁₃₈-SopE (SEQ ID No. 5 and 135) induced dramatic changes in the actin network (FIG. 5A). Similar results were obtained with another GEF effector protein, IpgB1 from Shigella flexneri (SEQ ID No. 4). Remarkably, first changes in the actin cytoskeleton were observed as fast as 2 min p.i. (FIG. 5A). Therefore, one can conclude that T3SS dependent protein delivery happens immediately after infection is initiated by centrifugation. To proof strict T3SS dependent transport, one of the T3SS proteins forming the translocation pore into the eukaryotic cell membrane was deleted (YopB, see [80]) (FIG. 12).

During Salmonella infection, SopE translocation is followed by translocation of SptP, which functions as a GTPase activating protein (GAP) for Cdc42 [81]. Whereas the translocation of YopE₁₋₁₃₈-SopE-Myc (SEQ ID No. 135) alone triggered massive F-actin rearrangements, the co-infection with YopE₁₋₁₃₈-SptP (SEQ ID No. 8) expressing bacteria abolished this effect in a dose dependent manner (FIG. 5B). An anti-Myc staining indicated that this inhibition was not due to a reduced level of YopE₁₋₁₃₈-SopE-Myc translocation (FIG. 5B). Together these results showed that the co-infection of cells with two bacterial strains is a valid method to deliver two different effectors into single cells to address their functional interaction.

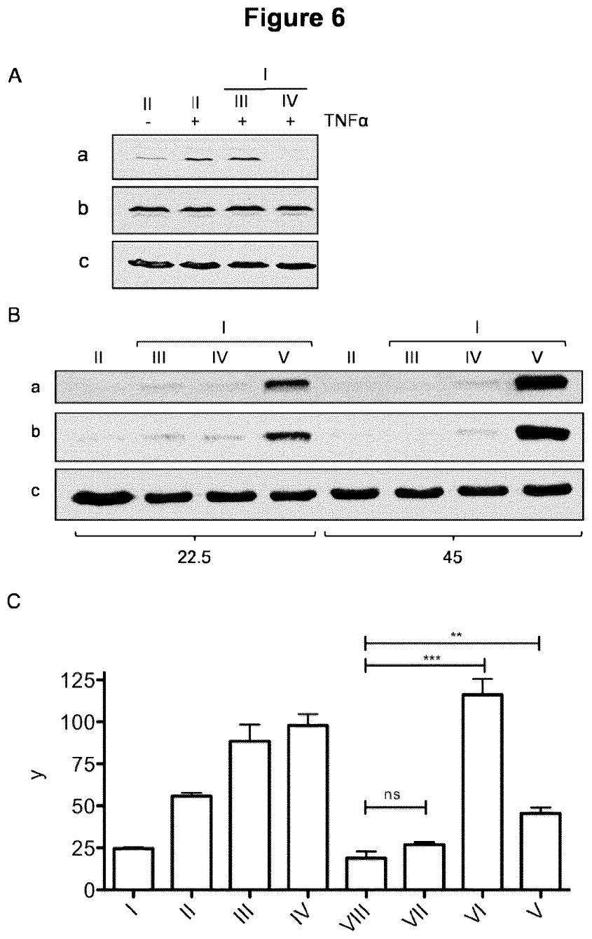

The S. flexneri type III effector OspF functions as a phosphothreonine lyase that dephosphorylates MAP kinases p38 and ERK [82]. To test the functionality of translocated YopE₁₋₁₃₈-OspF (SEQ ID No. 7), we monitored the phosphorylation of p38 after stimulation with TNFα. In uninfected cells or in cells infected with YopE₁₋₁₃₈-Myc expressing bacteria, TNFα□induced p38 phosphorylation. In contrast, after translocation of YopE₁₋₁₃₈-OspF, TNFα-induced phosphorylation was abolished, showing that the delivered OspF is active towards p38 (FIG. 6A).

During Salmonella infection, the type III effector SopB protects epithelial cells from apoptosis by sustained activation of Akt [83]. Whereas the translocation of YopE₁₋₁₃₈-Myc or YopE₁₋₁₃₈-SopE had no effect on Akt, the translocation of YopE₁₋₁₃₈-SopB (SEQ ID No. 6) induced a strong phosphorylation of Akt at T308 and S473, reflecting the active form (FIG. 6B). Similar results were obtained with the SopB-homolog from S. flexneri (IpgD, SEQ ID No. 9). Altogether, our results show that the YopE₁₋₁₃₈-based delivery system functions for all T3S effectors tested so far, and that it allows investigating proteins involved in the control of central cellular functions including the cytoskeleton, inflammation and cell survival.

A number of bacteria, including Agrobacterium tumefaciens, Legionella pneumophila and Bartonella henselae, use type IV secretion to inject effectors into cells. We tested whether the type IV effector BepA from B. henselae could be translocated into HeLa cells using our tool. Full length BepA (SEQ ID No. 10) and BepA_{E305-end} (SEQ ID No. 11) containing the C-terminal Bid domain, were cloned and cells were infected with the respective strains. As BepA was shown to induce the production of cyclic AMP (cAMP) [84], the level of cAMP in HeLa cells was measured after infection. Whereas the translocation of the Bid domain of the B. henselae effector BepG (SEQ ID No. 136) failed to induce cAMP, full length BepA and BepA_{E305-end} triggered cAMP production in expected amounts [84] (FIG. 6C). This result shows, that type IV effectors can also be effectively delivered by the YopE₁₋₁₃₈-based delivery system into host cell targets and that they are functional.

### Translocation of Eukaryotic Proteins Into Epithelial Cells

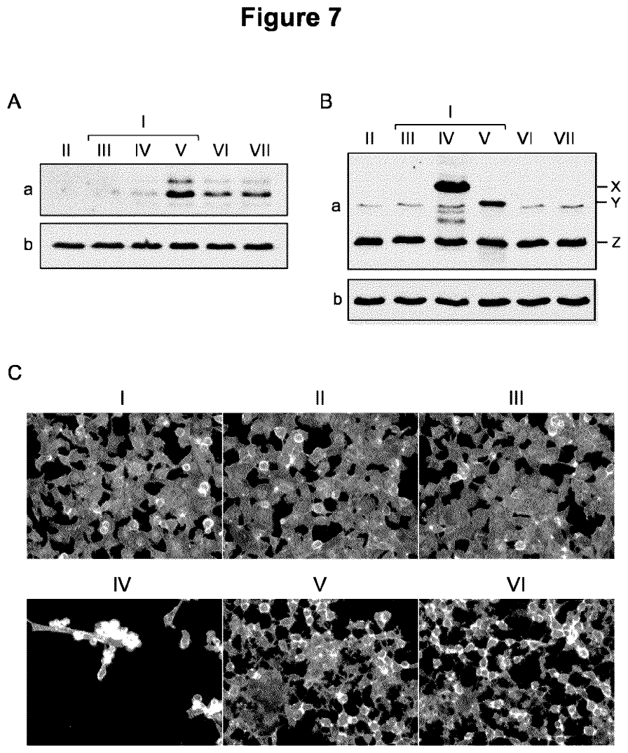

To show that human proteins can translocate via type III secretion we fused human apoptosis inducers for delivery by Y. enterocolitica to YopE₁₋₁₃₈ or for delivery by S. enterica to SteA₁₋₂₀, SteA, SopE₁₋₈₁ or SopE₁₋₁₀₅. We then monitored the translocation of the human BH3 interacting-domain death agonist (BID, SEQ ID No. 24), which is a pro-apoptotic member of the Bcl-2 protein family. It is a mediator of mitochondrial damage induced by caspase-8 (CASP8). CASP8 cleaves BID, and the truncated BID (tBID, SEQ ID No. 25) translocates to mitochondria where it triggers cytochrome c release. The latter leads to the intrinsic mode of caspase 3 (CASP3) activation during which it is cleaved into 17 and 12 kDa subunits [85]. Whereas infection for 1 h with YopE₁₋₁₃₈-Myc or YopE₁₋₁₃₈-BID expressing Y. enterocolitica failed to induce apoptosis, the translocation of human tBID triggered cell death in larger extend than the well-characterized apoptosis inducer staurosporin (FIGS. 7A and C). As expected, the translocation of tBID lead to the production of CASP3 p17 subunit, even in larger amounts as with staurosporin (FIG. 7A). To be able to compare translocated protein amounts to endogenous Bid, HeLa cells were lysed with Digitonin and analyzed by Western blotting using an anti Bid antibody (FIG. 7B). T3SS delivered YopE₁₋₁₃₈-tBID reached about endogenous Bid levels in HeLa cells, while delivered YopE₁₋₁₃₈-BID was present in even higher quantities (2.5 fold) (FIG. 7B). A deep proteome and transcriptome mapping of HeLa cells estimated 4.4 fold 10⁵ copies of BID per single cell [86]. Therefore, one can conclude that T3SS dependent human protein delivery reaches 10⁵ to 10⁶ proteins per cell. These numbers fit the copies per cell of nanobodies translocated via E. coli T3SS [19]. Assuming a levelling of a factor of 10 for the MOI and for the duration of the infection, a factor of 3.2 for the time-point of antibiotic addition and for the culture time at 37° C. before infection, the delivered protein copies/cell can be tuned from some 1000 copies/cell up to some 10⁶ copies/cell Altogether, these results indicated that translocated tBID was functional and delivered at relevant levels. This validated the translocation tool to study the role of proteins in the regulation of apoptosis, a central aspect of cell biology.

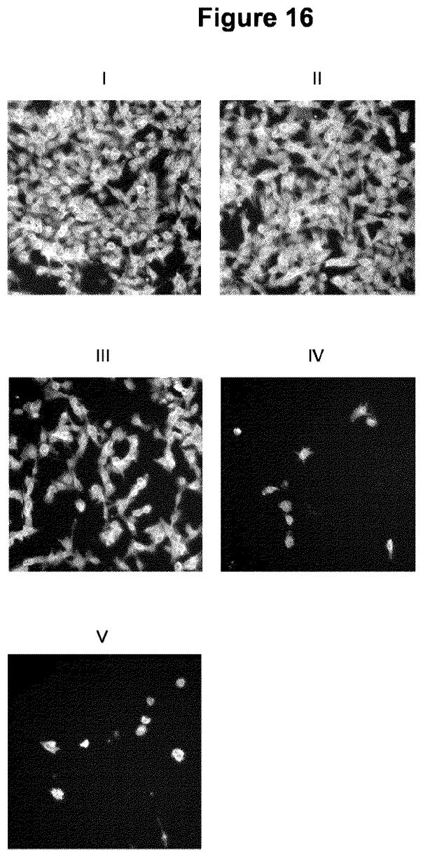

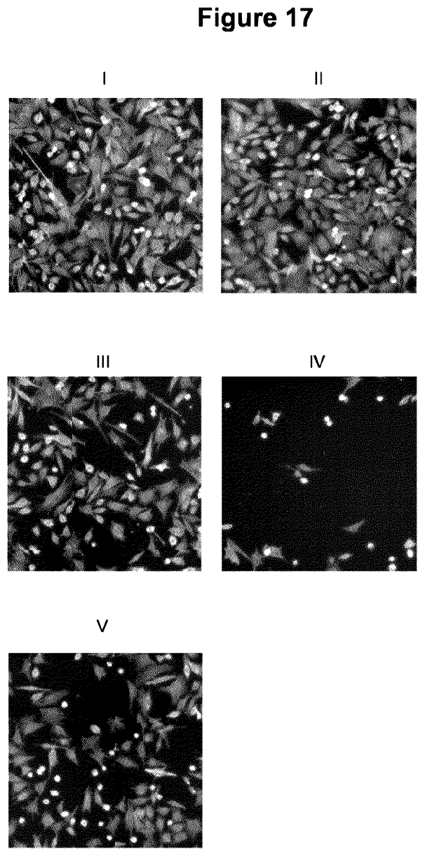

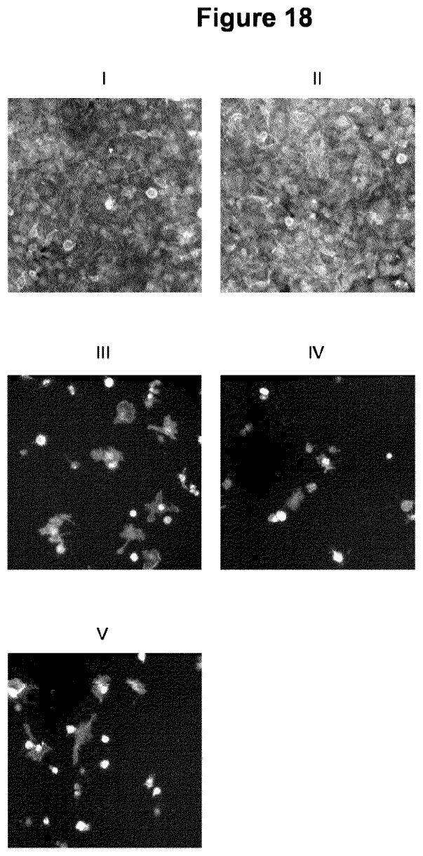

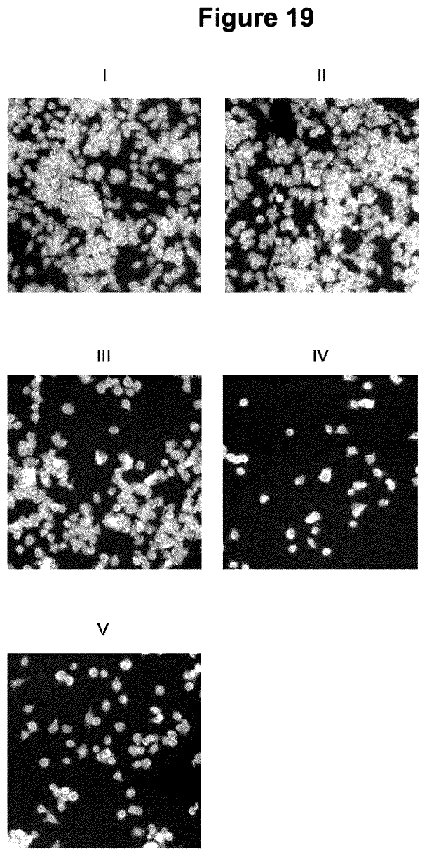

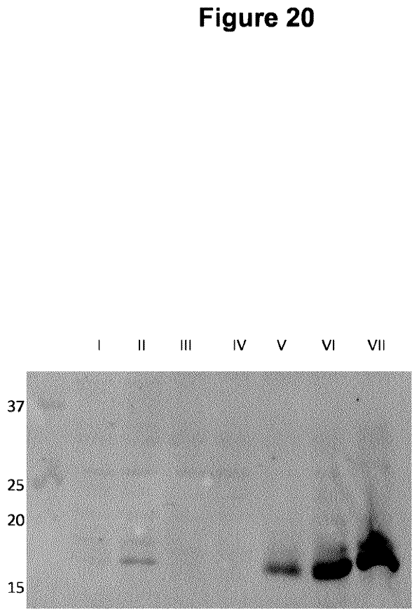

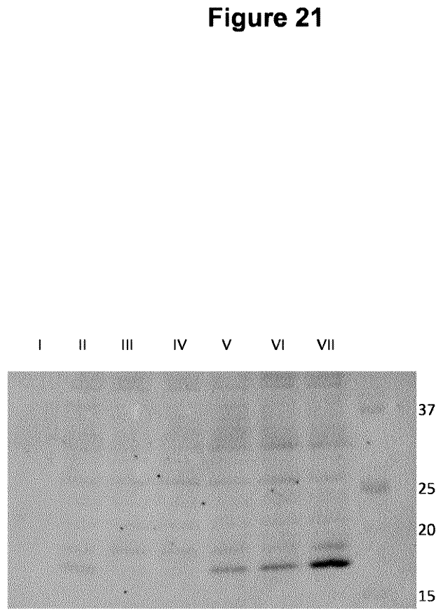

We further fused murine tBID (codon optimized for Y. enterocolitica; SEQ ID No. 194) or the BH3 domains of murine tBID or murine BAX (in both cases codon optimized for Y. enterocolitica; SEQ ID No. 138 and 139) to YopE₁₋₁₃₈ for delivery by Y. enterocolitica. Whereas infection for 2.5 h with Y. enterocolitica ΔHOPEMT asd delivering no protein or YopE₁₋₁₃₈-Myc failed to induce apoptosis, the translocation of murine tBID (codon optimized to Y. enterocolitica, SEQ ID No. 194) triggered cell death in B16F10 (FIG. 16), D2A1 (FIG. 17), HeLa (FIG. 18) and 4T1 (FIG. 19) cells. The translocation of the BH3 domain of murine BID codon optimized for Y. enterocolitica (SEQ ID 138) or murine BAX codon optimized for Y. enterocolitica (SEQ ID 139) were as well found to induce massive cell death in B16F10 (FIG. 16), D2A1 (FIG. 17), HeLa (FIG. 18) and 4T1 (FIG. 19) cells.

Whereas infection for 4 h with S. enterica aroA bacteria failed to induce apoptosis, the translocation of murine tBID triggered apoptosis, as the translocation of murine tBID lead to the production of CASP3 p17 subunit (FIGS. 20 and 21). The extent of apoptosis induction for SopE fusion proteins was larger when using SpiI T3SS inducing conditions (FIG. 20), which reflects the transport of SopE exclusively by SpiI T3SS. SteA₁₋₂₀ fused murine tBID failed to induce apoptosis, very likely because the secretion signal within the 20 N-terminal amino acids of SteA is not sufficient to allow delivery of a fusion protein (FIGS. 20 and 21). Murine tBID fused to full length SteA lead to apoptosis induction in HeLa cells (FIGS. 20 and 21), both in SpiI and SpiII T3SS inducing conditions, reflecting the ability of SteA to be transported by both T3SS. It has to be noted that even under SpiII T3SS inducing conditions, a partial activity of the SpiI T3SS is expected as seen by the activity of SopE fusion proteins in SpiII T3SS inducing conditions (FIG. 21).

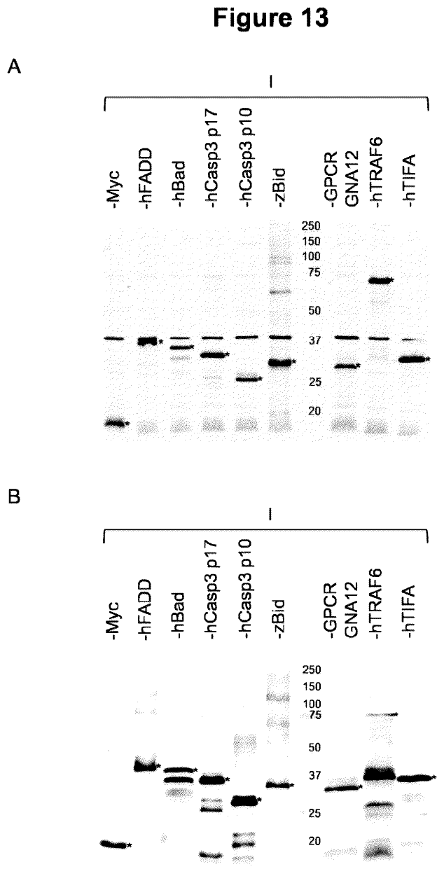

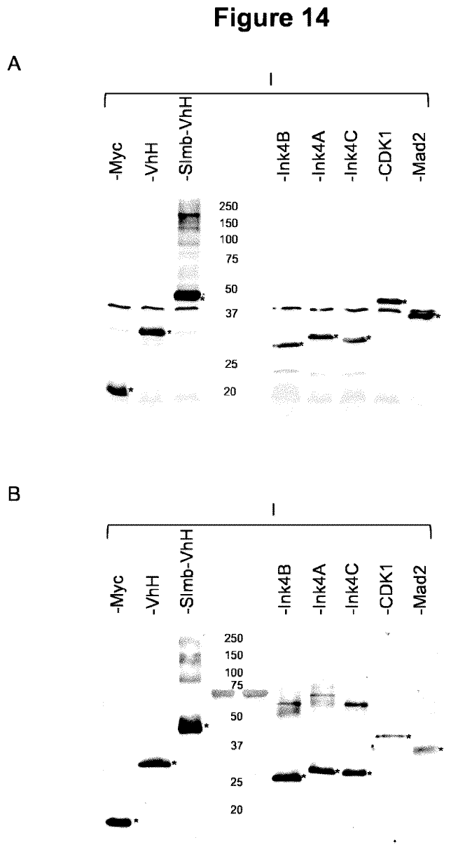

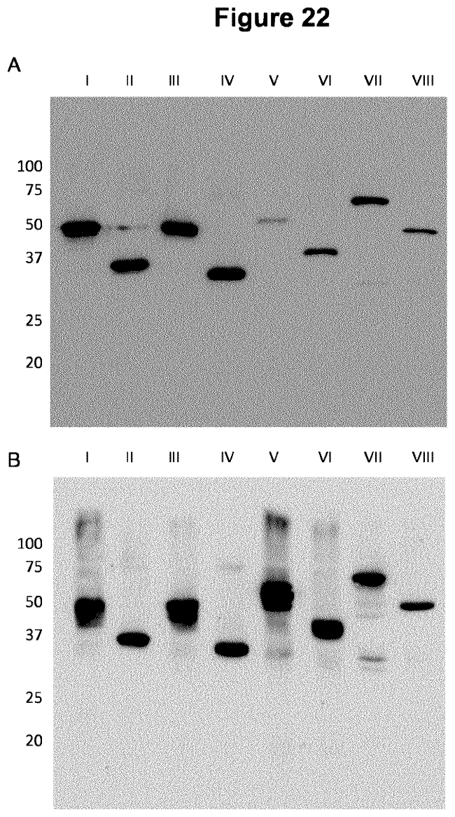

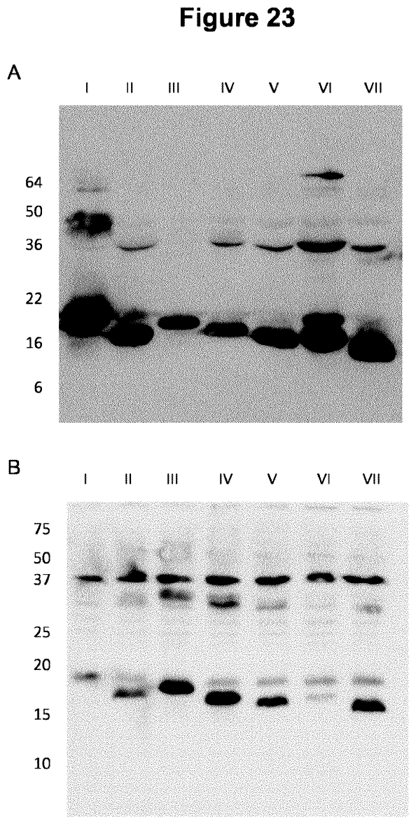

Besides the here functionally elaborated translocated eukaryotic proteins, several other eukaryotic proteins have been secreted using the here-described tool. This includes for delivery by Y. enterocolitica (FIGS. 13, 14 and 23) proteins from cell cycle regulation (Mad2 (SEQ ID No. 15), CDK1 (SEQ ID No. 14), INK4A (SEQ ID No. 16), INK4B (SEQ ID No. 17) and INK4C (SEQ ID No. 18)) as well as parts thereof (INK4A 84-103 (SEQ ID No. 158), p107 657-662 (SEQ ID No. 159), p21 141-160 (SEQ ID No. 160), p21 145-160 (SEQ ID No. 161), p21 17-33 (SEQ ID No. 162) and cyclin D2 139-147 (SEQ ID No 163)), apoptosis related proteins (Bad (SEQ ID No. 29), FADD (SEQ ID No. 28), and Caspase 3 p17 (SEQ ID No. 22) and p12 (SEQ ID No. 23), zebrafish Bid (SEQ ID No. 19) and t-Bid (SEQ ID No. 20)) as well as parts thereof (tBid BH3 (SEQ ID No.138), Bax BH3 (SEQ ID No.139)), signalling proteins (murine TRAF6 (SEQ ID No. 12), TIFA (SEQ ID No. 13)), GPCR Gα subunit (GNA12, shortest isoform, (SEQ ID No. 30)), nanobody (vhhGFP4, (SEQ ID No. 31)) and nanobody fusion constructs for targeted protein degradation (Slmb-vhhGFP4; (SEQ_ID_Nos. 32, 33, 34) [87]) (FIGS. 13 and 14) as well as small GTPases (Rac1 Q61E (SEQ ID No. 26 and 137) and RhoA Q63L (SEQ ID No. 27) and Pleckstrin homology domain from human Akt (SEQ ID No. 35). Besides the functionally elaborated apoptosis related proteins (murine tBid, SEQ ID No. 144-147), this further includes for delivery by S. enterica (FIG. 22) proteins from cell cycle regulation (Mad2 (SEQ ID No. 168-169), CDK1 (SEQ ID No. 170-171), INK4A (SEQ ID No. 164-165) and INK4C (SEQ ID No. 166-167)). While those proteins have not been functionally validated, the possibility of T3SS dependent secretion of diverse eukaryotic proteins in combination with the possible removal of the YopE appendage opens up new vistas on the broad applicability of T3SS in cell biology.

### In Vivo Translocation of Truncated Bid in Zebrafish Embryos Induces Apoptosis

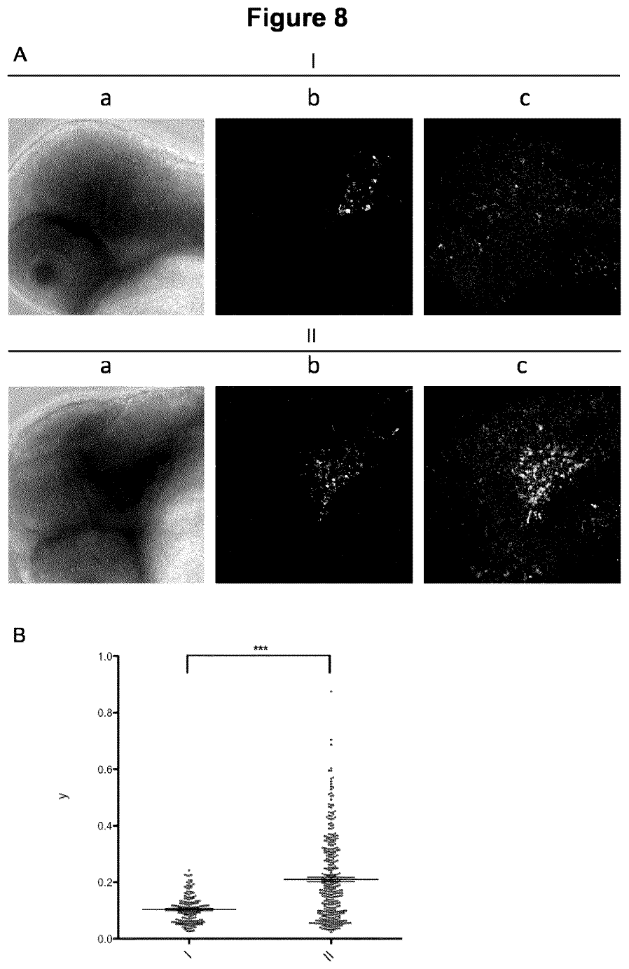

An interesting feature of this bacterial tool is the potential use in living animals. Zebrafish in their embryonic state can be kept transparent allowing fluorescent staining and microscopy [54, 88, 89]. Few zebrafish apoptosis inducers have been described in detail, whereof z-BIM is the most potent [90]. Therefore, we decided to clone z-BIM into our system. Even if weakly homolgous to human BIM, we assayed the potency of apoptosis induction of YopE₁₋₁₃₈-z-BIM (SEQ ID No. 21) in human epithelial cells. HeLa cells infected for 1 h with the strain translocating YopE₁₋₁₃₈-z-BIM showed clear signs of cell death. We then performed in-vivo experiments with 2 days post fertilization (dpf) zebrafish embryos, using a localized infection model via microinjection of bacteria into the hindbrain [54]. After infection for 5.5 h the fish were fixed, permeabilized and stained for presence of CASP3 p17. Upon infection with the YopE₁₋₁₃₈-Myc expressing strain, bacteria were visible in the hindbrain region (staining “b”, FIG. 8A I) but no induction of apoptosis around the bacteria was detected (staining “c”, FIG. 8A I). In contrast, upon infection with the strain delivering YopE₁₋₁₃₈-z-BIM a strong increase in presence of cleaved CASP3 was observed in regions surrounding the bacteria (FIG. 8A II). Automated image analysis on maximum intensity z projections confirms that YopE₁₋₁₃₈-z-BIM translocating bacteria induce apoptosis in nearby cells by far more than control bacteria do (FIG. 8B). This indicates that z-BIM is functional in zebrafish upon bacterial translocation. These results further validate the use of T3SS for eukaryotic protein delivery in living animals.

### Phosphoproteomics Reveal the Global Impact of Translocated Proteins on Protein Phosphorylation

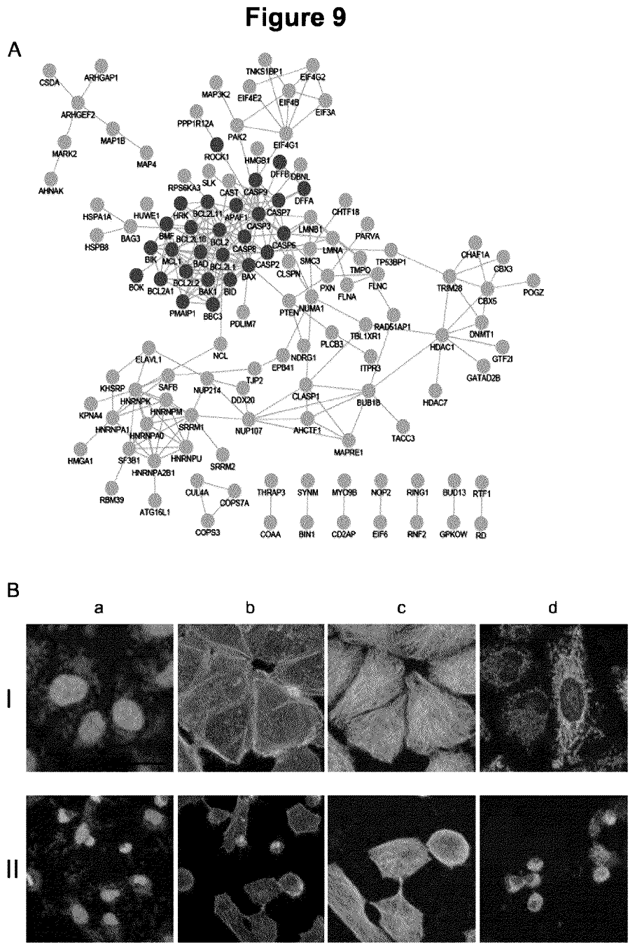

Phosphorylation is a wide-spread post-translational modification which can either activate or inactivate biological processes and is therefore a suitable target to study signaling events [91, 92]. Despite this, no systems-level analysis of phosphorylation in apoptosis is available today. To analyze the impact of human tBid delivered into Hela cells, we used a label-free phosphoproteomic approach by LC-MS/MS. In three independent experiments, cells were either left untreated, infected with ΔHOPEMT asd+YopE₁₋₁₃₈-Myc or with ΔHOPEMT asd+YopE₁₋₁₃₈-tBid for 30 minutes. Cells were lysed, followed by enzymatic digestion, phosphopetide enrichment and quantification and identification of individual phosphpeptides. We compared cells infected with ΔHOPEMT asd+YopE₁₋₁₃₈-Myc to cells infected with ΔHOPEMT asd+YopE₁₋₁₃₈-tBid, allowing us to identify 363 tBid dependent phosphorylation events. 286 phosphopeptides showed an increase in phosphorylation whereas 77 were less phosphorylated upon tBid delivery, corresponding to 243 different proteins, which we defined as the tBid phosphoproteome. The STRING database was used to create a protein-protein interaction network of the tBid phosphoproteome [93] (FIG. 9A). Additonally 27 proteins known to be related to mitochondrial apoptosis were added to the network, building a central cluster. Interestingly, only few proteins from the tBid phosphoproteome are connected to this central cluster indicating that many proteins undergo a change in phosphorylation that were so far not directly linked to apoptotic proteins. To characterize the biological functions covered by the tBid phosphoproteome, we performed a gene ontology analysis using the functional annotation tool of the Database for Annotation, Visualization, and Integrated Discovery (DAVID, http:/david.abec.nciforf.gov/) [94, 95]. Identified biological functions show that diverse cellular processes are affected by tBid. Many proteins involved in chromatin rearrangement and the regulation of transcription undergo a change in phosphorylation (i.e. CBX3, CBX5, TRIM28, HDAC1). HDAC1 for example is a histone deacetylase playing a role in regulation of transcription. It has been shown that HDAC1 can modulate transcriptional activity of NF-kB, a protein also participating in apoptosis. We additionally identified a cluster of proteins involved in RNA processing which has previously been shown to play an important role in the regulation of apoptosis [96]. HNRPK for instance mediates a p53/TP53 response to DNA damage and is necessary for the induction of apoptosis [97]. Furthermore, the phosphorylation of proteins involved in protein translation is also affected. Several eukaryotic initiation factors (i.e. EIF4E2, EIF4B, EIF3A, EIF4G2) undergo a change in phosphorylation, which is in line with the observation that overall protein synthesis is decreased in apoptotic cells. Interestingly, the phosphorylation of many proteins involved in cytoskeleton remodeling (e.g. PXN, MAP1B9 are altered upon tBid delivery. This is in concordance with the observation that the morphology of cells changes dramatically upon tBid delivery (FIG. 9B). Cells shrinkage and loss of contact is reflected by the fact that we observe phosphorylation of adhesion related proteins like ZO2 and Paxillin. Similarly, shrinkage of the nuclei is accompanied by phosphorylation of laminar proteins like LaminA/C and Lamin B1. Altogether, tBID delivery induces a rapid apoptotic response also indicated by rupture of the mitochondrial integrity (FIG. 9B). We showed that tBid induced apoptosis affects hundreds of phosphorylation events participating in diverse cellular processes. While many identified proteins have been related to apoptosis, only few were known to be phosphorylated upon apoptosis induction. The phosphoproteomic approach thus provides a useful resource for further studies on apoptosis.

### LIST OF REFERENCES

1. Gibson, T.J., M. Seiler, and R.A. Veitia (2013) The transience of transient overexpression. Nat Methods. 10: 715-21.
2. Inoue, T., W.D. Heo, J.S. Grimley, T.J. Wandless, and T. Meyer (2005) An inducible translocation strategy to rapidly activate and inhibit small GTPase signaling pathways. Nat Methods. 2: 415-8.
3. Pust, S., H. Hochmann, E. Kaiser, G. von Figura, K. Heine, et al. (2007) A cell-permeable fusion toxin as a tool to study the consequences of actin-ADP-ribosylation caused by the Salmonella enterica virulence factor SpvB in intact cells. J Biol Chem. 282: 10272-82.
4. Hayes, C.S., S.K. Aoki, and D.A. Low (2010) Bacterial contact-dependent delivery systems. Annu Rev Genet. 44: 71-90.
5. Cornelis, G.R. (2006) The type III secretion injectisome. Nat Rev Microbiol. 4: 811-25.
6. Michiels, T., P. Wattiau, R. Brasseur, J.M. Ruysschaert, and G. Cornelis (1990) Secretion of Yop proteins by Yersiniae. Infect Immun. 58: 2840-9.
7. Letzelter, M., I. Sorg, L.J. Mota, S. Meyer, J. Stalder, et al. (2006) The discovery of SycO highlights a new function for type III secretion effector chaperones. EMBO J. 25: 3223-33.
8. Gauthier, A., and B.B. Finlay (2003) Translocated intimin receptor and its chaperone interact with ATPase of the type III secretion apparatus of enteropathogenic Escherichia coli. J Bacteriol. 185: 6747-55.
9. Wattiau, P., and G.R. Cornelis (1993) SycE, a chaperone-like protein of Yersinia enterocolitica involved in the secretion of YopE. Mol Microbiol. 8: 123-31.
10. Feldman, M.F., S. Muller, E. Wuest, and G.R. Cornelis (2002) SycE allows secretion of YopE-DHFR hybrids by the Yersinia enterocolitica type III Ysc system. Mol Microbiol. 46: 1183-97.
11. Akeda, Y., and J.E. Galan (2005) Chaperone release and unfolding of substrates in type III secretion. Nature. 437: 911-5.
12. Pais, S.V., C. Milho, F. Almeida, and L.J. Mota (2013) Identification of novel type III secretion chaperone-substrate complexes of Chlamydia trachomatis. PLOS One. 8: e56292.
13. Sory, M.P., and G.R. Cornelis (1994) Translocation of a hybrid YopE-adenylate cyclase from Yersinia enterocolitica into HeLa cells. Mol Microbiol. 14: 583-94.
14. Garcia, J.T., F. Ferracci, M.W. Jackson, S.S. Joseph, I. Pattis, et al. (2006) Measurement of effector protein injection by type III and type IV secretion systems by using a 13-residue phosphorylatable glycogen synthase kinase tag. Infect Immun. 74: 5645-57.
15. Chen, L.M., G. Briones, R.O. Donis, and J.E. Galan (2006) Optimization of the delivery of heterologous proteins by the Salmonella enterica serovar Typhimurium type III secretion system for vaccine development. Infect Immun. 74: 5826-33.
16. Russmann, H., H. Shams, F. Poblete, Y. Fu, J.E. Galan, et al. (1998) Delivery of epitopes by the Salmonella type III secretion system for vaccine development. Science. 281: 565-8.
17. Russmann, H., U. Gerdemann, E.I. Igwe, K. Panthel, J. Heesemann, et al. (2003) Attenuated Yersinia pseudotuberculosis carrier vaccine for simultaneous antigen-specific CD4 and CD8 T-cell induction. Infect Immun. 71: 3463-72.
18. Chaux, P., R. Luiten, N. Demotte, V. Vantomme, V. Stroobant, et al. (1999) Identification of five MAGE-A1 epitopes recognized by cytolytic T lymphocytes obtained by in vitro stimulation with dendritic cells transduced with MAGE-A1. J Immunol. 163: 2928-36.
19. Blanco-Toribio, A., S. Muyldermans, G. Frankel, and L.A. Fernandez (2010) Direct injection of functional single-domain antibodies from E. coli into human cells. PLoS One. 5: e15227.
20. Bichsel, C., D. Neeld, T. Hamazaki, L.J. Chang, L.J. Yang, et al. (2013) Direct reprogramming of fibroblasts to myocytes via bacterial injection of MyoD protein. Cell Reprogram. 15: 117-25.
21. Bichsel, C., D.K. Neeld, T. Hamazaki, D. Wu, L.J. Chang, et al. (2011) Bacterial delivery of nuclear proteins into pluripotent and differentiated cells. PLoS One. 6: e 16465.
22. Chamekh, M., A. Phalipon, R. Quertainmont, I. Salmon, P. Sansonetti, et al. (2008) Delivery of biologically active anti-inflammatory cytokines IL-10 and IL-1ra in vivo by the Shigella type III secretion apparatus. J Immunol. 180: 4292-8.
23. Hoffman, R.M. (2011) Tumor-seeking Salmonella amino acid auxotrophs. Curr Opin Biotechnol. 22: 917-23.
24. Hoang, T.T., S. Williams, H.P. Schweizer, and J.S. Lam (1997) Molecular genetic analysis of the region containing the essential Pseudomonas aeruginosa asd gene encoding aspartate-beta-semialdehyde dehydrogenase. Microbiology. 143 (Pt 3): 899-907.
25. Skurnik, M., and H. Wolf-Watz (1989) Analysis of the yopA gene encoding the Yop1 virulence determinants of Yersinia spp. Mol Microbiol. 3: 517-29.
26. Tertti, R., M. Skurnik, T. Vartio, and P. Kuusela (1992) Adhesion protein YadA of Yersinia species mediates binding of bacteria to fibronectin. Infect Immun. 60: 3021-4.
27. Isberg, R.R., and J.M. Leong (1990) Multiple beta 1 chain integrins are receptors for invasin, a protein that promotes bacterial penetration into mammalian cells. Cell. 60: 861-71.
28. Isberg, R.R., D.L. Voorhis, and S. Falkow (1987) Identification of invasin: a protein that allows enteric bacteria to penetrate cultured mammalian cells. Cell. 50: 769-78.
29. Leong, J.M., R.S. Fournier, and R.R. Isberg (1990) Identification of the integrin binding domain of the Yersinia pseudotuberculosis invasin protein. EMBO J. 9: 1979-89.
30. Mota, L.J., and G.R. Cornelis (2005) The bacterial injection kit: type III secretion systems. Ann Med. 37: 234-49.
31. Trosky, J.E., A.D. Liverman, and K. Orth (2008) Yersinia outer proteins: Yops. Cell Microbiol. 10: 557-65.
32. Brenner, D., and T.W. Mak (2009) Mitochondrial cell death effectors. Curr Opin Cell Biol. 21: 871-7.
33. Chalah, A., and R. Khosravi-Far (2008) The mitochondrial death pathway. Adv Exp Med Biol. 615: 25-45.
34. Fuchs, Y., and H. Steller (2011) Programmed cell death in animal development and disease. Cell. 147: 742-58.
35. Waugh, D.S. (2011) An overview of enzymatic reagents for the removal of affinity tags. Protein Expr Purif. 80: 283-93.
36. Sarker, M.R., C. Neyt, I. Stainier, and G.R. Cornelis (1998) The Yersinia Yop virulon: LcrV is required for extrusion of the translocators YopB and YopD. J Bacteriol. 180: 1207-14.
37. Ramamurthi, K.S., and O. Schneewind (2005) A synonymous mutation in Yersinia enterocolitica yopE affects the function of the YopE type III secretion signal. J Bacteriol. 187:707-15.
38. Wolke, S., N. Ackermann, and J. Heesemann (2011) The Yersinia enterocolitica type 3 secretion system (T3SS) as toolbox for studying the cell biological effects of bacterial Rho GTPase modulating T3SS effector proteins. Cell Microbiol. 13:1339-57.
39. Forsberg, A., and H. Wolf-Watz (1990) Genetic analysis of the yopE region of Yersinia spp.: identification of a novel conserved locus, yerA, regulating yopE expression. J Bacteriol. 172:1547-55.
40. Sambrook, J. 2001. Molecular cloning: a laboratory manual. D.W. Russell, editor. Cold Spring Harbor Laboratory Press, Cold Spring Harbor, N.Y.
41. Alto, N.M., and J.E. Dixon (2008) Analysis of Rho-GTPase mimicry by a family of bacterial type III effector proteins. Methods Enzymol. 439:131-43.
42. Alto, N.M., F. Shao, C.S. Lazar, R.L. Brost, G. Chua, et al. (2006) Identification of a bacterial type III effector family with G protein mimicry functions. Cell. 124:133-45.
43. Kaniga, K., I. Delor, and G.R. Cornelis (1991) A wide-host-range suicide vector for improving reverse genetics in gram-negative bacteria: inactivation of the blaA gene of Yersinia enterocolitica. Gene. 109:137-41.
44. Yoneda, Y., T. Semba, Y. Kaneda, R.L. Noble, Y. Matsuoka, et al. (1992) A long synthetic peptide containing a nuclear localization signal and its flanking sequences of SV40 T-antigen directs the transport of IgM into the nucleus efficiently. Exp Cell Res. 201:313-20.
45. Cornelis, G.R. 1997. Cross talk between Yersinia and eukaryotic cells. In Molecular aspects of host-pathoge interactions. S. MoCRAE, SMYTH, STOW, editor. Cambridge University Press.
46. Metcalf, W.W., W. Jiang, and B.L. Wanner (1994) Use of the rep technique for allele replacement to construct new Escherichia coli hosts for maintenance of R6K gamma origin plasmids at different copy numbers. Gene. 138:1-7.
47. Diepold, A., M. Amstutz, S. Abel, I. Sorg, U. Jenal, et al. (2010) Deciphering the assembly of the Yersinia type III secretion injectisome. EMBO J. 29:1928-40.
48. Iriarte, M., I. Stainier, and G.R. Cornelis (1995) The rpoS gene from Yersinia enterocolitica and its influence on expression of virulence factors. Infect Immun. 63:1840-7.
49. Cornelis, G., J.C. Vanootegem, and C. Sluiters (1987) Transcription of the yop regulon from Y. enterocolitica requires trans acting pYV and chromosomal genes. Microb Pathog. 2:367-79.
50. Grosdent, N., I. Maridonneau-Parini, M.P. Sory, and G.R. Cornelis (2002) Role of Yops and adhesins in resistance of Yersinia enterocolitica to phagocytosis. Infect Immun. 70:4165-76.
51. Dehio, C., M. Meyer, J. Berger, H. Schwarz, and C. Lanz (1997) Interaction of Bartonella henselae with endothelial cells results in bacterial aggregation on the cell surface and the subsequent engulfment and internalisation of the bacterial aggregate by a unique structure, the invasome. J Cell Sci. 110 (Pt 18): 2141-54.
52. Westerfield, M. (2000) The Zebrafish Book: A Guide for the Laboratory Use of Zebrafish Danio rerio University of Oregon Press, Eugene, ORp.
53. Kimmel, C.B., W.W. Ballard, S.R. Kimmel, B. Ullmann, and T.F. Schilling (1995) Stages of embryonic development of the zebrafish. Dev Dyn. 203:253-310.
54. Benard, E.L., A.M. van der Sar, F. Ellett, G.J. Lieschke, H.P. Spaink, et al. (2012) Infection of zebrafish embryos with intracellular bacterial pathogens. J Vis Exp.
55. Blum, Y., H.G. Belting, E. Ellertsdottir, L. Herwig, F. Luders, et al. (2008) Complex cell rearrangements during intersegmental vessel sprouting and vessel fusion in the zebrafish embryo. Dev Biol. 316:312-22.
56. Herwig, L., Y. Blum, A. Krudewig, E. Ellertsdottir, A. Lenard, et al. (2011) Distinct cellular mechanisms of blood vessel fusion in the zebrafish embryo. Curr Biol. 21:1942-8.
57. Carpenter, A.E., T.R. Jones, M.R. Lamprecht, C. Clarke, I.H. Kang, et al. (2006) CellProfiler: image analysis software for identifying and quantifying cell phenotypes. Genome Biol. 7: R100.
58. Bensimon, A., A. Schmidt, Y. Ziv, R. Elkon, S.Y. Wang, et al. (2010) ATM-dependent and -independent dynamics of the nuclear phosphoproteome after DNA damage. Sci Signal. 3: rs3.
59. Perkins, D.N., D.J. Pappin, D.M. Creasy, and J.S. Cottrell (1999) Probability-based protein identification by searching sequence databases using mass spectrometry data. Electrophoresis. 20: 3551-67.
60. Smyth, G.K. (2004) Linear models and empirical bayes methods for assessing differential expression in microarray experiments. Stat Appl Genet Mol Biol. 3:Article3.
61. Ting, L., M.J. Cowley, S.L. Hoon, M. Guilhaus, M.J. Raftery, et al. (2009) Normalization and statistical analysis of quantitative proteomics data generated by metabolic labeling. Mol Cell Proteomics. 8: 2227-42.
62. Vizcaino, J.A., R.G. Cote, A. Csordas, J.A. Dianes, A. Fabregat, et al. (2013) The PRoteomics IDEntifications (PRIDE) database and associated tools: status in 2013. Nucleic Acids Res. 41: D1063-9.
63. Boyd, A.P., I. Lambermont, and G.R. Cornelis (2000) Competition between the Yops of Yersinia enterocolitica for delivery into eukaryotic cells: role of the SycE chaperone binding domain of YopE. J Bacteriol. 182: 4811-21.
64. Iriarte, M., and G.R. Cornelis (1998) YopT, a new Yersinia Yop effector protein, affects the cytoskeleton of host cells. Mol Microbiol. 29: 915-29.
65. Kudryashev, M., M. Stenta, S. Schmelz, M. Amstutz, U. Wiesand, et al. (2013) In situ structural analysis of the Yersinia enterocolitica injectisome. Elife. 2: e00792.
66. Schulte, R., G.A. Grassl, S. Preger, S. Fessele, C.A. Jacobi, et al. (2000) Yersinia enterocolitica invasin protein triggers IL-8 production in epithelial cells via activation of Rel p65-p65 homodimers. FASEB J. 14: 1471-84.
67. Mota, L.J., L. Journet, I. Sorg, C. Agrain, and G.R. Cornelis (2005) Bacterial injectisomes: needle length does matter. Science. 307: 1278.
68. Isaksson, E.L., M. Aili, A. Fahlgren, S.E. Carlsson, R. Rosqvist, et al. (2009) The membrane localization domain is required for intracellular localization and autoregulation of YopE in Yersinia pseudotuberculosis. Infect Immun. 77: 4740-9.
69. Denecker, G., S. Totemeyer, L.J. Mota, P. Troisfontaines, I. Lambermont, et al. (2002) Effect of low-and high-virulence Yersinia enterocolitica strains on the inflammatory response of human umbilical vein endothelial cells. Infect Immun. 70: 3510-20.
70. Sharma, S., A. Hirabuchi, K. Yoshida, K. Fujisaki, A. Ito, et al. (2013) Deployment of the Burkholderia glumae type III secretion system as an efficient tool for translocating pathogen effectors to monocot cells. Plant J. 74: 701-12.
71. Carrington, J.C., and W.G. Dougherty (1988) A viral cleavage site cassette: identification of amino acid sequences required for tobacco etch virus polyprotein processing. Proc Natl Acad Sci U S A. 85: 3391-5.
72. Kapust, R.B., J. Tozser, T.D. Copeland, and D.S. Waugh (2002) The P1′ specificity of tobacco etch virus protease. Biochem Biophys Res Commun. 294: 949-55.
73. Liang, H., H. Gao, C.A. Maynard, and W.A. Powell (2005) Expression of a self-processing, pathogen resistance-enhancing gene construct in Arabidopsis. Biotechnol Lett. 27: 435-42.
74. Weber, W., C. Fux, M. Daoud-el Baba, B. Keller, C.C. Weber, et al. (2002) Macrolide-based transgene control in mammalian cells and mice. Nat Biotechnol. 20: 901-7.
75. Kapust, R.B., J. Tozser, J.D. Fox, D.E. Anderson, S. Cherry, et al. (2001) Tobacco etch virus protease: mechanism of autolysis and rational design of stable mutants with wild-type catalytic proficiency. Protein Eng. 14: 993-1000.
76. Lee, V.T., D.M. Anderson, and O. Schneewind (1998) Targeting of Yersinia Yop proteins into the cytosol of HeLa cells: one-step translocation of YopE across bacterial and eukaryotic membranes is dependent on SycE chaperone. Mol Microbiol. 28: 593-601.
77. Gray, D.C., S. Mahrus, and J.A. Wells (2010) Activation of specific apoptotic caspases with an engineered small-molecule-activated protease. Cell. 142: 637-46.
78. Henrichs, T., N. Mikhaleva, C. Conz, E. Deuerling, D. Boyd, et al. (2005) Target-directed proteolysis at the ribosome. Proc Natl Acad Sci U S A. 102: 4246-51.
79. Hardt, W.D., L.M. Chen, K.E. Schuebel, X.R. Bustelo, and J.E. Galan (1998) S. typhimurium encodes an activator of Rho GTPases that induces membrane ruffling and nuclear responses in host cells. Cell. 93: 815-26.
80. Hakansson, S., K. Schesser, C. Persson, E.E. Galyov, R. Rosqvist, et al. (1996) The YopB protein of Yersinia pseudotuberculosis is essential for the translocation of Yop effector proteins across the target cell plasma membrane and displays a contact-dependent membrane disrupting activity. EMBO J. 15: 5812-23.
81. Stebbins, C.E., and J.E. Galan (2001) Structural mimicry in bacterial virulence. Nature. 412: 701-5.
82. Li, H., H. Xu, Y. Zhou, J. Zhang, C. Long, et al. (2007) The phosphothreonine lyase activity of a bacterial type III effector family. Science. 315: 1000-3.
83. Norris, F.A., M.P. Wilson, T.S. Wallis, E.E. Galyov, and P.W. Majerus (1998) SopB, a protein required for virulence of Salmonella dublin, is an inositol phosphate phosphatase. Proc Natl Acad Sci U S A. 95: 14057-9.
84. Pulliainen, A.T., K. Pieles, C.S. Brand, B. Hauert, A. Bohm, et al. (2012) Bacterial effector binds host cell adenylyl cyclase to potentiate Galphas-dependent cAMP production. Proc Natl Acad Sci U S A. 109: 9581-6.
85. Li, H., H. Zhu, C.J. Xu, and J. Yuan (1998) Cleavage of BID by caspase 8 mediates the mitochondrial damage in the Fas pathway of apoptosis. Cell. 94: 491-501.
86. Nagaraj, N., J.R. Wisniewski, T. Geiger, J. Cox, M. Kircher, et al. (2011) Deep proteome and transcriptome mapping of a human cancer cell line. Mol Syst Biol. 7: 548.
87. Caussinus, E., O. Kanca, and M. Affolter (2011) Fluorescent fusion protein knockout mediated by anti-GFP nanobody. Nat Struct Mol Biol. 19: 117-21.
88. Cosma, C.L., L.E. Swaim, H. Volkman, L. Ramakrishnan, and J.M. Davis (2006) Zebrafish and frog models of Mycobacterium marinum infection. Curr Protoc Microbiol. Chapter 10: Unit 10B 2.
89. Mathias, J.R., M.E. Dodd, K.B. Walters, S.K. Yoo, E.A. Ranheim, et al. (2009) Characterization of zebrafish larval inflammatory macrophages. Dev Comp Immunol. 33: 1212-7.
90. Jette, C.A., A.M. Flanagan, J. Ryan, U.J. Pyati, S. Carbonneau, et al. (2008) BIM and other BCL-2 family proteins exhibit cross-species conservation of function between zebrafish and mammals. Cell Death Differ. 15: 1063-72.
91. Olsen, J.V., B. Blagoev, F. Gnad, B. Macek, C. Kumar, et al. (2006) Global, in vivo, and site-specific phosphorylation dynamics in signaling networks. Cell. 127: 635-48.
92. Schmutz, C., E. Ahrne, C.A. Kasper, T. Tschon, I. Sorg, et al. (2013) Systems-Level Overview of Host Protein Phosphorylation During Shigella flexneri Infection Revealed by Phosphoproteomics. Mol Cell Proteomics. 12: 2952-68.
93. Szklarczyk, D., A. Franceschini, M. Kuhn, M. Simonovic, A. Roth, et al. (2011) The STRING database in 2011: functional interaction networks of proteins, globally integrated and scored. Nucleic Acids Res. 39: D561-8.
94. Huang da, W., B.T. Sherman, and R.A. Lempicki (2009) Bioinformatics enrichment tools: paths toward the comprehensive functional analysis of large gene lists. Nucleic Acids Res. 37: 1-13.
95. Huang da, W., B.T. Sherman, R. Stephens, M.W. Baseler, H.C. Lane, et al. (2008) DAVID gene ID conversion tool. Bioinformation. 2: 428-30.
96. Schwerk, C., and K. Schulze-Osthoff (2005) Regulation of apoptosis by alternative pre-mRNA splicing. Mol Cell. 19: 1-13.
97. Papagiannakopoulos, T., A. Shapiro, and K.S. Kosik (2008) MicroRNA-21 targets a network of key tumor-suppressive pathways in glioblastoma cells. Cancer Res. 68: 8164-72.
98. Hoiseth, S.K., B.A. Stocker (1981) Aromatic-dependent Salmonella typhimurium are non-virulent and effective as live vaccines. Nature 291:238-239.

1.-34. (canceled)

35. A recombinant Yersinia strain, wherein the Yersinia strain is transformed with a vector which comprises in the 5′ to 3′ direction:
a promoter;
a first DNA sequence encoding a delivery signal from a bacterial T3SS effector protein, operably linked to said promoter, wherein the delivery signal comprises a YopE effector protein or an N-terminal fragment thereof; and
a second DNA sequence, encoding a heterologous protein, fused in frame to the 3′end of said first DNA sequence, wherein the heterologous protein is selected from the group consisting of: proteins involved in apoptosis or apoptosis regulation, cell cycle regulators, ankyrin repeat proteins, cell signaling proteins, reporter proteins, transcription factors, proteases, GPCR related proteins, nanobody fusion constructs and nanobodies, bacterial T3SS effectors, and bacterial T4SS effectors.

36. The recombinant Yersinia strain of claim 35, wherein the vector comprises a third DNA sequence encoding a protease cleavage site, wherein the third DNA sequence is located between the 3′end of said first DNA sequence and the 5′end of said second DNA sequence.

37. The recombinant Yersinia strain of claim 35, wherein the Yersinia strain is wild type or deficient in the production of at least one T3SS effector protein and wherein the N-terminal fragment of the YopE effector protein comprises the N-terminal 138 amino acids of the Yersinia enterocolitica YopE effector protein.

38. The recombinant Yersinia strain of claim 35, wherein the Yersinia strain is deficient to produce adhesion proteins binding to a eukaryotic cell surface or extracellular matrix.

39. The recombinant Yersinia strain of claim 35, wherein the proteins involved in apoptosis or apoptosis regulation are selected from the group consisting of BH3-only proteins, caspases and intracellular signaling proteins of death receptor control of apoptosis.

40. The recombinant Yersinia strain of claim 35, wherein the vector comprises two second DNA sequences encoding identical or two different heterologous proteins fused independently from each other in frame to the 3′end of said first DNA sequence.

41. The recombinant Yersinia strain of claim 40, wherein both heterologous proteins are proteins involved in apoptosis or apoptosis regulation and wherein one protein is a pro-apoptotic protein and the other protein is an inhibitor of apoptosis-prevention pathways or wherein one protein is a pro-apoptotic protein and the other protein is an inhibitor of pro-survival signaling or pathways.

42. A vector which comprises in the 5′ to 3′ direction:
a promoter;
a first DNA sequence encoding a delivery signal from a bacterial T3SS effector protein, operably linked to said promoter, wherein the delivery signal comprises a YopE effector protein or an N-terminal fragment thereof; and
a second DNA sequence, encoding a heterologous protein, fused in frame to the 3′end of said first DNA sequence, wherein the heterologous protein is selected from the group consisting of: proteins involved in apoptosis or apoptosis regulation, cell cycle regulators, ankyrin repeat proteins, cell signalling proteins, reporter proteins, transcription factors, proteases, GPCR related proteins, nanobody fusion constructs and nanobodies, bacterial T3SS effectors, and bacterial T4SS effectors.

43. The vector of claim 42, wherein the vector comprises a third DNA sequence encoding a protease cleavage site, wherein the third DNA sequence is located between the 3′end of said first DNA sequence and the 5′end of said second DNA sequence.

44. The vector of claim 42, wherein the N-terminal fragment of the YopE effector protein comprises the N-terminal 138 amino acids of the Yersinia enterocolitica YopE effector protein.

45. The vector of claim 42, wherein the proteins involved in apoptosis or apoptosis regulation are selected from the group consisting of BH3-only proteins, caspases and intracellular signaling proteins of death receptor control of apoptosis.

46. The vector of claim 42, wherein the vector comprises two second DNA sequences encoding identical or two different heterologous proteins fused independently from each other in frame to the 3′end of said first DNA sequence.

47. The vector of claim 46, wherein both heterologous proteins are proteins involved in apoptosis or apoptosis regulation and wherein one protein is a pro-apoptotic protein and the other protein is an inhibitor of apoptosis-prevention pathways or wherein one protein is a pro-apoptotic protein and the other protein is an inhibitor of pro-survival signaling or pathways.

48. A method for delivering a heterologous protein into a eukaryotic cell comprising the following steps:
i) culturing the recombinant Yersinia strain of claim 35; and
ii) contacting the eukaryotic cell with the recombinant Yersinia strain of i),
wherein a fusion protein which comprises a delivery signal from the bacterial T3SS effector protein and the heterologous protein is expressed by the recombinant Yersinia strain and is translocated into the eukaryotic cell.

49. A method of purifying a heterologous protein, the method comprising:
culturing the recombinant Yersinia strain of claim 35 so that a fusion protein which comprises the delivery signal from the bacterial T3SS effector protein and the heterologous protein is expressed and secreted into the supernatant of the culture.

50. A method of delivering a heterologous protein as a medicament or as a vaccine to a subject, the method comprising contacting the subject with the recombinant Yersinia strain of claim 35.
